(19) United States
(12) Patent Application Publication
(10) Pub. No.: US 2026/0209180 A1
(43) Pub. Date: Jul. 23, 2026

(54) PROTEASE INHIBITORS FOR TREATING OR PREVENTING CORONAVIRUS INFECTION

(71) Applicant: Merck Sharp & Dohme LLC, Rahway, NJ (US)

(72) Inventors: Michael J. Kelly, III, Paoli, PA (US); Mark E. Layton, Harleysville, PA (US); John J. Acton, III, Cranford, NJ (US); Mayuri Gupta, Union, NJ (US); Franca-Maria Klingler, Letchworth Garden City (GB); Harini Krishnamurthy, Doylestown, PA (US); John A. McCauley, Maple Glen, PA (US); Gregori J. Morriello, Randolph, NJ (US); Craig A. Parish, Tenafly, NJ (US); James J. Perkins, Churchville, PA (US); Manuel de Lera Ruiz, Perkasie, PA (US); Valerie W. Shurtleff, Philadelphia, PA (US); Anthony J. Roecker, Santee, CA (US)

(73) Assignee: Merck Sharp & Dohme LLC, Rahway, NJ (US)

(21) Appl. No.: 19/145,012

(22) PCT Filed: Jan. 4, 2024
(86) PCT No.: PCT/US2024/010237

§ 371 Date: Jul. 1, 2025

Related U.S. Application Data
(60) Provisional application No. 63/437,768, filed on Jan. 9, 2023.

(51) Int. Cl. C07D 213/81; A61K 31/167; A61K 31/17; A61K 31/325; A61K 31/351; A61K 31/352; A61K 31/403; A61K 31/426; A61K 31/427; A61K 31/428; A61K 31/44; A61K 31/454; A61K 31/519; A61K 31/53; A61K 31/7068; A61P 31/14; C07C 237/22; C07C 237/42; C07C 271/20; C07C 271/28; C07C 275/40; C07D 205/04; C07D 213/82; C07D 213/85; C07D 277/30; C07D 277/60; C07D 309/08; C07D 311/96; C07D 487/04

(52) U.S. Cl. CPC C07D 213/81; A61K 31/167; A61K 31/17; A61K 31/325; A61K 31/351; A61K 31/352; A61K 31/403; A61K 31/426; A61K 31/427; A61K 31/428; A61K 31/44; A61K 31/454; A61K 31/519; A61K 31/53; A61K 31/7068; A61P 31/14; C07C 237/22; C07C 237/42; C07C 271/20; C07C 271/28; C07C 275/40; C07D 205/04; C07D 213/82; C07D 213/85; C07D 277/30; C07D 277/60; C07D 309/08; C07D 311/96; C07D 487/04; C07C 2601/02; C07C 2601/14; C07C 2602/38; C07C 2602/50

(57) ABSTRACT

The present invention provides a compound of Formula I wherein R₁, R₂, R₃, R₄, R₅, R₆, R₇, and subscripts X and n are as described herein and pharmaceutical compositions comprising one or more said compounds, and methods for using said compounds for the treatment, inhibition, or amelioration of one or more disease states that could benefit from inhibition of a coronavirus, including SARS-CoV, MERS-CoV and SARS-CoV-2. The compounds of this invention could further be used in combination with other therapeutically effective agents, including but not limited to, other drugs useful for the treatment of coronavirus infection. The invention furthermore relates to processes for preparing compounds of Formula I, and pharmaceutical compositions which comprise compounds of Formula I and pharmaceutically acceptable salts thereof.

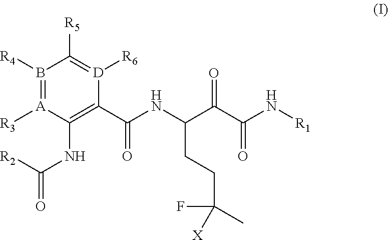

### CROSS REFERENCE TO RELATED APPLICATIONS

The present application claims the benefit of U.S. Provisional Application No. 63/437,768, filed Jan. 9, 2023, hereby incorporated by reference in its entirety.

### FIELD OF THE INVENTION

The present invention relates to certain protease inhibitors, pharmaceutical compositions comprising such inhibitors, and methods for using said compounds for the treatment, inhibition or amelioration of one or more disease states that could benefit from inhibition of a coronavirus, including SARS-CoV, MERS-CoV and SARS-CoV-2.

### BACKGROUND OF THE INVENTION

Coronaviruses (CoVs) are large, enveloped, positive-stranded, RNA viruses that comprise the Coronavirinae subfamily in the Nirovirales order. CoVs are further classified into four genera: alpha coronavirus, beta coronavirus, gamma coronavirus and delta coronavirus. Alpha and beta CoVs infect humans and other mammals, whereas the gamma and delta CoVs infect only animals (e.g., birds, sea mammals, pigs). CoV infection can result in a wide range of acute to chronic diseases of the respiratory, enteric and central nervous systems (Fields Virology Emerging Viruses Vol. 1. 2021. pp. 410-412).

To date, seven different coronaviruses that cause disease in humans have been identified: HCoV-229E, HCoV-NL63, HCoV-OC43, HCoV-HKU1, severe acute respiratory syndrome coronavirus (SARS-CoV), Middle East respiratory syndrome coronavirus (MERS-CoV) and, most recently SARS-CoV-2. HCOV-229E, HCoV-NL63, HCoV-OC43 and HCoV-HKU1 circulate on a yearly basis and cause mild symptoms similar to a common cold (Forni D, Cagliani R, Clerici M, and Sironi M. 2017. Trends in Microbiology, January 2017, Vol. 25, No. 1. 35-48). SARS-CoV, MERS-CoV and SARS-CoV-2 however, which have emerged in three zoonotic CoV transmission events over the last 21 years, are associated with mild to severe symptoms of respiratory infection such as fever, cough, dyspnea, pneumonia and acute respiratory distress syndrome that can ultimately lead to death.

The SARS-CoV epidemic in 2002 to 2003 was contained, but it resulted in 8,000 SARS-CoV infections and more than 800 deaths (Fields Virology Emerging Viruses Vol. 1. 2021. pp. 438). Camel-human zoonotic transmission of MERS-CoV occurred in Saudi Arabia in 2012. Although human to human transmission has been documented, most de novo infections occur as a result of camel-human interactions, and outbreaks are generally localized to the Arabian Peninsula (Zaki A M, van Zaki A M, van Boheemen S, Bestebroer T M, Osterhaus A, Fouchier R A M. 2012 N Engl J Med 367:1814-1820). The fatality rate of MERS infection is about 36% (www.who.int/csr/don/16-october-2014-mers/en/). SARS-CoV-2, the pandemic strain causal of COVID-19, is of bat origin, and transmission from bat to humans may have occurred directly or via an unknown intermediate host animal (Lu R, Zhao X, Li J, et al. 2020. Lancet; 395 (10224): 565-574). SARS-CoV-2 is now a pandemic CoV and has resulted, as of December 2021, in a worldwide health and economic crisis with global deaths exceeding 5 million (JHU CSSE COVID-19 Data github.com/CSSEGISandData/COVID-19). These three well-characterized zoonotic events, and the likelihood of future spillover events with novel CoVs, underscores the need for broad-spectrum CoV antiviral therapies that will be active against both existing CoVs, such as MERS-CoV and SARS-CoV-2, and also CoVs that may emerge in the future.

CoV particles consist of a cell-derived lipid membrane containing structural proteins spike (S), membrane (M), envelope (E), and nucleocapsid (N) (Fields Virology Emerging Viruses Vol. 1 2021 pp. 416-417). The virion also contains a large (25-32 kb) non-segmented positive-sense single-strand viral RNA genome that, similar to cellular mRNAs, is 5′-capped, contains 5′ and 3′ untranslated regions (UTRs) and a 3′ polyadenylated tail. All CoV viral genomes contain six basic common genes: two long open reading frames (1a and 1b) that encode two polypeptides that constitute the non-structural proteins (nsps) that form the multiprotein replicase-transcription complex (RTC) and four open reading frames for the structural proteins S, M, E and N that make up the virion. Depending on the CoV, one to eight additional genes, called accessory genes, can be encoded in the genome. The genomic organization amongst all CoVs is conserved and invariant across different genera such that the gene sequence is always 1a, 1b, S, M, E and N.

CoV replication is initiated through binding of the S protein to a specific cell surface receptor. SARS-CoV and SARS-CoV-2, for example, engage the angiotensin converting enzyme 2 (ACE-2) on cells of the upper respiratory tract (Lu R, Zhao X, Li J, et al. 2020. Lancet; 395 (10224): 565-574). Viral attachment leads to either viral endocytosis followed by fusion of the viral and endosome membranes, or direct fusion of the viral and cellular plasma members at the cell surface, to release virions into the cytoplasm. After entry, the viral genomic RNA is uncoated and serves as a template for cap-dependent translation of Orf 1a and Orf 1b to produce the viral polypeptides pp 1a and pp 1ab (Fung S, Liu D, 2019. Annu. Rev. Microbiol. 73:529-57). Cleavage of the viral polypeptides to yield the individual replisome proteins is carried out by the viral papain-like protease (PLPro or nsp3) and 3CL main protease (Mpro or nsp5). The nsps form double-membraned vesicles and assemble to form RTCs responsible for genome replication, sub-genomic RNA (sgRNA) synthesis and transcription of the sgRNAs. The sgRNA serve as templates from which the mRNAs encoding for the structural and accessory proteins are translated. Assembly of new viral particles occurs in the endoplasmic reticulum-golgi intermediate complex and mature particles are released through secretory vesicles.

Vaccines for prevention of COVID-19 have been developed using the S protein of SARS-CoV-2 as an antigen to elicit a protective immune response (Kryikidis et. al. npj Vaccines 28 (2021) 6:28). Vaccines based on mRNA/lipid nanoparticle and replication-defective adenoviruses vectored platforms have both been demonstrated to be highly effective for prevention of serious illness. However, there is limited data on the effectiveness of these vaccines for transmission of SARS-CoV-2. A liability of using the S protein for vaccine development is that the amino acid sequence is highly variable, enabling the SARS-CoV-2 to adapt to immune pressure (Chen R E et al. Nature Medicine. Mar. 4, 2021). Multiple independent spike mutations have been detected, even in the absence of vaccine selective pressure, and some variants will likely lead to reduced efficacy in vaccine clinical trials conducted where those variants are circulating.

Given the limitations of the current vaccines and the potential for zoonotic emergence of new pandemic strains, there is an urgent need for broad-spectrum anti-coronaviral treatment and prophylactic regimens. An anti-coronavirus intervention with efficacy against SARS-CoV, SARS-CoV-2, and the more distantly related MERS-CoV would be expected to have broad-spectrum activity against both SARS-CoV-2 and future CoVs that may emerge through zoonotic events.

### SUMMARY OF THE INVENTION

The present invention provides compounds of Formula I:

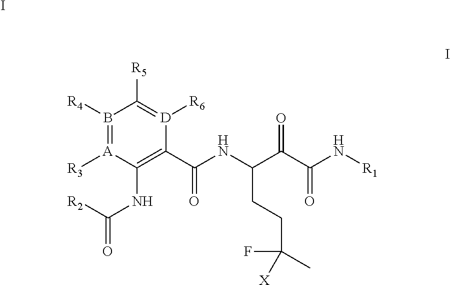

and pharmaceutically acceptable salts thereof. The compounds of Formula I are protease inhibitors, and as such may be useful in the treatment, inhibition, or amelioration of one or more disease states that could benefit from inhibition of a coronavirus, including SARS-CoV, MERS-CoV and SARS-CoV-2. Thus, the present invention also provides a method for prophylaxis or treatment of a coronavirus infection (e.g., a SARS-CoV, a SARS-CoV-2 or a MERS-CoV infection), comprising administering an effective amount of the compound of any of the compounds of Formula I disclosed herein or a pharmaceutically acceptable salt thereof to a patient in need thereof.

The compounds of this invention could further be used in combination with other therapeutically effective agents (one or more additional therapeutic agents), including but not limited to, other drugs useful for the treatment of coronavirus infection. Such additional therapeutic agents could include molnupiravir, pomotrelvir, ensitrelvir, nirmatrelvir, and ritonavir. The invention furthermore relates to processes for preparing compounds of Formula I, and pharmaceutical compositions which comprise compounds of Formula I and pharmaceutically acceptable salts thereof.

### DETAILED DESCRIPTION OF THE INVENTION

In one aspect, the present invention a compound of Formula I:

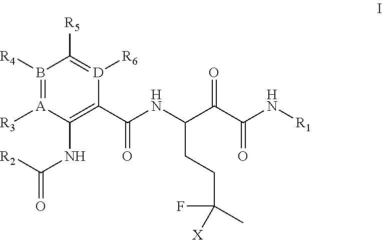

or a pharmaceutically acceptable salt thereof, wherein:
R₁ is H, (C₃-C₆)cycloalkyl, (C₁-C₆)alkyl, (C₁-C₆)alkyl-OH, phenyl, (C₁-C₆)alkyl-phenyl, (C₄-C₆) heterocycloalkyl containing 1 to 3 hetereoatom(s) independently selected from N, O, or S, (C₁-C₆)alkyl-(C₄-C₆) heterocycloalkyl containing 1 to 3 heteroatom(s) independently selected from N, O, or S, (C₁-C₆)alkyl-(C₅-C₆) heteroaryl containing 1 to 3 heteroatom(s) independently selected from N, O, or S, (C₅-C₆) heteroaryl containing 1 to 3 heteroatom(s) independently selected from N, O, or S;
R₂ is (C₁-C₆)alkyl; (C₁-C₆)alkyl-CF₃; (C₁-C₆)alkyl-OH; (C₁-C₆)alkyl-O—CH₃; (C₁-C₆)alkyl-O—CF₃; (C₁-C₆)alkyl-O—(C₃-C₁₀)cycloalkyl optionally substituted by an OH, —(C₁-C₆)alkyl, CF₃, (C₁-C₆)alkyl-CF₃, or up to 3 halogen; (CF₂)-phenyl; (C₁-C₆)alkyl-phenyl; (C₃-C₁₀)cycloalkyl optionally substituted by an OH, —(C₁-C₆)alkyl, CF₃, —(C₁-C₆)alkyl-CF₃, or up to 3 halogen; (C₁-C₆)alkyl-(C₃-C₁₀)cycloalkyl optionally substituted by a OH, —(C₁-C₆)alkyl, CF₃, —(C₁-C₆)alkyl-CF₃, or up to 3 halogen; (C₄-C₁₀) heterocycle including up to 3 heteroatoms independently selected from N, O, and S and optionally substituted by a carbonyl, OH, —(C₁-C₆)alkyl, CF₃, —(C₁-C₆)alkyl-CF₃, or up to 3 halogen; (C₁-C₆)alkyl-(C₄-C₁₀) heterocycle including up to 3 heteroatoms independently selected from N, O, and S and optionally substituted by a carbonyl, OH, —(C₁-C₆)alkyl, CF₃, —(C₁-C₆)alkyl-CF₃, or up to 3 halogen; (C₆-C₁₀) aryl optionally substituted by an OH, —(C₁-C₆)alkyl, CF₃, —(C₁-C₆)alkyl-CF₃, or up to 3 halogen; (C₁-C₆)alkyl-(C₆-C₁₀) aryl optionally substituted by an OH, —(C₁-C₆)alkyl, CF₃, —(C₁-C₆)alkyl-CF₃, or up to 3 halogen; (C₅-C₁₀) heteroaryl including up to 4 heteroatoms independently selected from N, O, and S and optionally substituted by an OH, CN, CHF₂, —(C₁-C₆)alkyl, CF₃, —(C₁-C₆)alkyl-CF₃, —(C₃-C₆)cycloalkyl, O—(C₁-C₆)alkyl, O—CF₃, O—(C₃-C₆)cycloalkyl, or up to 3 halogen; (C₁-C₆)alkyl-(C₅-C₁₀) heteroaryl including up to 4 heteroatoms independently selected from N, O, and S and optionally substituted by an OH, CN, CHF₂, (C₃-C₆)cycloalkyl, O—(C₃-C₆)cycloalkyl, —(C₁-C₆)alkyl, O—(C₁-C₆)alkyl, CF₃, O—CF₃, —(C₁-C₆)alkyl-CF₃, or up to 3 halogen; —O—(C₁-C₆)alkyl; —O—(C₁-C₆)alkyl-(C₃-C₆)cycloalkyl optionally substituted by an OH, CF₃, —(C₁-C₆)alkyl, —(C₁-C₆)alkyl-CF₃, or up to 3 halogen; —O—(C₃-C₆)cycloalkyl optionally substituted by an OH, CF₃, —(C₁-C₆)alkyl, —(C₁-C₆)alkyl-CF₃, or up to 3 halogen; (C₁-C₆)alkyl-N—CHO—O(C₁-C₆)alkyl; CHF₂; CF₃; or N(R₇)₂
R₃ is H, F, Cl, or (C₁-C₆)alkyl;
R₄ is H, F, Cl, or (C₁-C₆)alkyl;
R₅ is H, F, Cl, CN, CF₃, O—CHF₂, O—CF₃, —(C₁-C₆)alkyl-CF₃, CHF₂, CF₃, (C₁-C₆)alkyl, (C₃-C₆)cycloalkyl, —O—(C₃-C₆)cycloalkyl, or O—(C₁-C₆)alkyl;
R₆ is H, F, Cl, or (C₁-C₆)alkyl;
R₇ is independently H; (C₁-C₆)alkyl; (C₃-C₁₀)cycloalkyl optionally substituted by an OH, —(C₁-C₆)alkyl, CF₃, —(C₁-C₆)alkyl-CF₃, or up to 3 halogen; (C₁-C₆)alkyl-(C₃-C₁₀)cycloalkyl optionally substituted by an OH, —(C₁-C₆)alkyl, CF₃, —(C₁-C₆)alkyl-CF₃, or up to 3 halogen; (C₄-C₁₀) heterocycle including up to 3 heteroatoms selected from N, O, and S and optionally substituted by an OH, —(C₁-C₆)alkyl, CF₃, —(C₁-C₆)alkyl-CF₃, or up to 3 halogen; (C₁-C₆)alkyl-(C₄-C₁₀) heterocycle including up to 3 heteroatoms selected from N, O, and S and optionally substituted by an OH, —(C₁-C₆)alkyl, CF₃, —(C₁-C₆)alkyl-CF₃, or up to 3 halogen; (C₆-C₁₀) aryl optionally substituted by an OH, —(C₁-C₆)alkyl, CF₃, —(C₁-C₆)alkyl-CF₃, or up to 3 halogen; (C₁-C₆)alkyl-(C₆-C₁₀) aryl optionally substituted by an OH, —(C₁-C₆)alkyl, CF₃, —(C₁-C₆)alkyl-CF₃, or up to 3 halogen; (C₅-C₁₀) heteroaryl including up to 3 heteroatoms selected from N, O, and S and optionally substituted by an OH, —(C₁-C₆)alkyl, CF₃, —(C₁-C₆)alkyl-CF₃, or up to 3 halogen; (C₁-C₆)alkyl-(C₅-C₁₀) heteroaryl including up to 3 heteroatoms selected from N, O, and S and optionally substituted by an OH, —(C₁-C₆)alkyl, CF₃, —(C₁-C₆)alkyl-CF₃, or up to 3 halogen;
A is C or N;
B is C or N,
D is C or N, provided that only 1 of A, B, or D can be N and that if A, B, or D is N then the respective R₃, R₄, or R₆ is absent;
X is H or F.

In some embodiments of the present invention, R₁ is H, (C₃-C₆)cycloalkyl, or (C₁-C₆)alkyl. In specific embodiments, R₁ is H, CH₃ or cyclopropyl.

In some embodiments of the present invention, the group R₂ is CH₃,

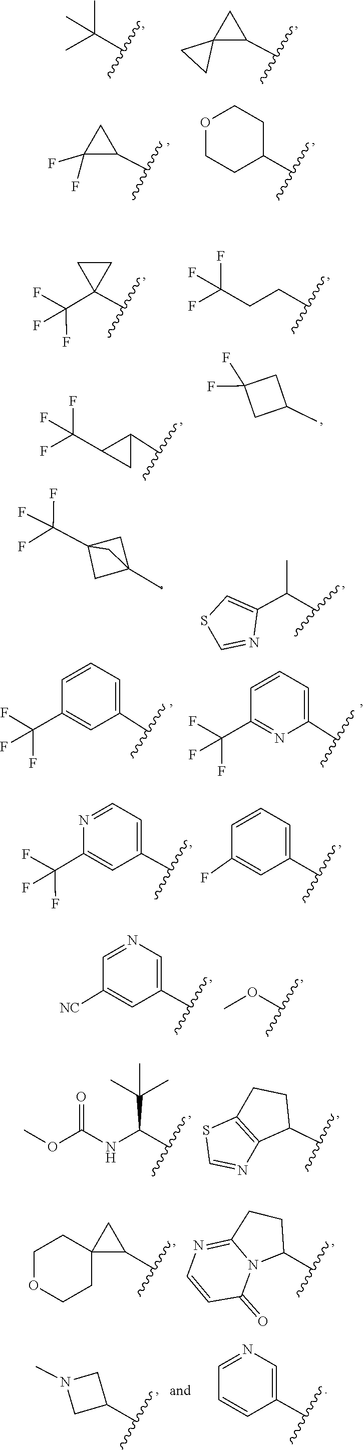

In certain embodiments of the present invention, R₃ is H, F, or Cl.

In some embodiments of the present invention, R₄ is H, F, or Cl.

In some embodiments of the present invention, R₅ is F, Cl, CHF₂, or CN.

In certain embodiments of the present invention, R₆ is H, F, or Cl.

In certain embodiments of the present invention, one of A, B or D is N.

In other embodiments of the present invention, A, B and D are all C.

In certain embodiments of the present invention, X is F.

In other embodiments of the present invention, X is H.

Reference to the specific classes and subclasses set forth above is meant to include all combinations of particular and preferred groups unless stated otherwise.

Specific embodiments of the present invention include, but are not limited to, the compounds disclosed in Examples 1 to 54, or pharmaceutically acceptable salts thereof.

Other specific embodiments include compounds enumerated below or pharmaceutically acceptable salts thereof:
N-[4-chloro-2-[[(1S)-4,4-difluoro-1-[2-(methylamino)-2-oxo-acetyl]pentyl]carbamoyl]phenyl]-2-(trifluoromethyl)pyridine-4-carboxamide;
5-chloro-N-[(1S)-4,4-difluoro-1-[2-(methylamino)-2-oxo-acetyl]pentyl]-2-[[3-(trifluoromethyl)bicyclo[1.1.1]pentane-1-carbonyl]amino]pyridine-3-carboxamide;
methyl N-[(1S)-1-[[4-chloro-2-[[(1S)-1-[2-(cyclopropylamino)-2-oxo-acetyl]-4,4-difluoro-pentyl]carbamoyl]phenyl]carbamoyl]-2,2-dimethyl-propyl]carbamate;
5-chloro-N-[(1S)-4,4-difluoro-1-[2-(methylamino)-2-oxo-acetyl]pentyl]-4-fluoro-2-(4,4,4-trifluorobutanoylamino)benzamide;
5-chloro-N-[(1S)-1-[2-(cyclopropylamino)-2-oxo-acetyl]-4,4-difluoro-pentyl]-2-[[3-(trifluoromethyl)benzoyl]amino]pyridine-3-carboxamide;
methyl N-[4-chloro-2-[[(1S)-4-fluoro-1-[2-(methylamino)-2-oxo-acetyl]pentyl]carbamoyl]phenyl]carbamate;
methyl N-[4-chloro-2-[[(1S)-4-fluoro-1-[2-(methylamino)-2-oxo-acetyl]pentyl]carbamoyl]phenyl]carbamate;
methyl N-[4-chloro-2-[[(1S)-4-fluoro-1-[2-(methylamino)-2-oxo-acetyl]pentyl]carbamoyl]phenyl]carbamate;
methyl N-[4-chloro-2-[[(1S)-4-fluoro-1-[2-(methylamino)-2-oxo-acetyl]pentyl]carbamoyl]phenyl]carbamate;
N-[4-chloro-2-[[(1S)-4,4-difluoro-1-oxamoyl-pentyl]carbamoyl]phenyl]-2-(trifluoromethyl)pyridine-4-carboxamide;
5-chloro-N-[(1S)-4,4-difluoro-1-[2-(methylamino)-2-oxo-acetyl]pentyl]-2-(2-thiazol-4-ylpropanoylamino)benzamide;
N-[4-chloro-2-[[(1S)-4,4-difluoro-1-[2-(methylamino)-2-oxo-acetyl]pentyl]carbamoyl]phenyl]-5,6-dihydro-4H-cyclopenta[d]thiazole-4-carboxamide;
5-chloro-N-[(1S)-4,4-difluoro-1-[2-(methylamino)-2-oxo-acetyl]pentyl]-2-(2-thiazol-4-ylpropanoylamino)benzamide;
N-[4-chloro-2-[[(1S)-4,4-difluoro-1-[2-(methylamino)-2-oxo-acetyl]pentyl]carbamoyl]phenyl]-6-oxaspiro[2.5]octane-2-carboxamide;
5-chloro-N-[(1S)-1-[2-(cyclopropylamino)-2-oxo-acetyl]-4,4-difluoro-pentyl]-2-[[3-(trifluoromethyl)benzoyl]amino]benzamide;
5-chloro-2-[(3,3-difluorocyclobutanecarbonyl)amino]-N-[(1S)-4,4-difluoro-1-[2-(methylamino)-2-oxo-acetyl]pentyl]benzamide;
5-chloro-N-[(1S)-4,4-difluoro-1-[2-(methylamino)-2-oxo-acetyl]pentyl]-2-(4,4,4-trifluorobutanoylamino)benzamide;
N-[4-chloro-2-[[(1S)-4,4-difluoro-1-[2-(methylamino)-2-oxo-acetyl]pentyl]carbamoyl]phenyl]-5,6-dihydro-4H-cyclopenta[d]thiazole-4-carboxamide;
N-[4-chloro-2-[[(1S)-4,4-difluoro-1-[2-(methylamino)-2-oxo-acetyl]pentyl]carbamoyl]phenyl]tetrahydropyran-4-carboxamide;
N-[4-chloro-2-[[(1S)-4,4-difluoro-1-[2-(methylamino)-2-oxo-acetyl]pentyl]carbamoyl]phenyl]-6-(trifluoromethyl)pyridine-2-carboxamide;
N-[(1S)-4,4-difluoro-1-[2-(methylamino)-2-oxo-acetyl]pentyl]-5-fluoro-2-[[3-(trifluoromethyl)benzoyl]amino]pyridine-3-carboxamide;
(2S)—N-[4-chloro-2-[[(1S)-4,4-difluoro-1-[2-(methylamino)-2-oxo-acetyl]pentyl]carbamoyl]phenyl]spiro[2.2]pentane-2-carboxamide;
5-chloro-N-[(1S)-4,4-difluoro-1-[2-(methylamino)-2-oxo-acetyl]pentyl]-2-[[1-(trifluoromethyl)cyclopropanecarbonyl]amino]benzamide;
5-chloro-N-[(1S)-4,4-difluoro-1-[2-(methylamino)-2-oxo-acetyl]pentyl]-2-[[3-(trifluoromethyl)benzoyl]amino]pyridine-3-carboxamide;
N-[4-chloro-2-[[(1S)-4,4-difluoro-1-[2-(methylamino)-2-oxo-acetyl]pentyl]carbamoyl]phenyl]-3-(trifluoromethyl)bicyclo[1.1.1]pentane-1-carboxamide;
5-chloro-2-[[(1R)-2,2-difluorocyclopropanecarbonyl]amino]-N-[(1S)-4,4-difluoro-1-[2-(methylamino)-2-oxo-acetyl]pentyl]benzamide;
5-chloro-N-[(1S)-4,4-difluoro-1-[2-(methylamino)-2-oxo-acetyl]pentyl]-2-[(3-fluorobenzoyl)amino]pyridine-3-carboxamide;
(6S)—N-[4-chloro-2-[[(1S)-4,4-difluoro-1-[2-(methylamino)-2-oxo-acetyl]pentyl]carbamoyl]phenyl]-4-oxo-7,8-dihydro-6H-pyrrolo[1,2-a]pyrimidine-6-carboxamide;
N-[4-chloro-2-[[(1S)-4,4-difluoro-1-[2-(methylamino)-2-oxo-acetyl]pentyl]carbamoyl]phenyl]-6-oxaspiro[2.5]octane-2-carboxamide;
5-chloro-N-[(1S)-1-[2-(cyclopropylamino)-2-oxo-acetyl]-4,4-difluoro-pentyl]-2-[[3-(trifluoromethyl)bicyclo[1.1.1]pentane-1-carbonyl]amino]pyridine-3-carboxamide;
2-[(3,3-difluorocyclobutanecarbonyl)amino]-N-[(1S)-4,4-difluoro-1-[2-(methylamino)-2-oxo-acetyl]pentyl]-5-fluoro-pyridine-3-carboxamide;
5-chloro-N-[(1S)-1-[2-(cyclopropylamino)-2-oxo-acetyl]-4,4-difluoro-pentyl]-2-[(3-fluorobenzoyl)amino]pyridine-3-carboxamide;
5-chloro-N-[(1S)-4,4-difluoro-1-[2-(methylamino)-2-oxo-acetyl]pentyl]-2-[[2-(trifluoromethyl)cyclopropanecarbonyl]amino]benzamide;
N-[(1S)-1-[2-(cyclopropylamino)-2-oxo-acetyl]-4,4-difluoro-pentyl]-5-fluoro-2-[[3-(trifluoromethyl)benzoyl]amino]pyridine-3-carboxamide;
5-chloro-N-[(1S)-4,4-difluoro-1-[2-(methylamino)-2-oxo-acetyl]pentyl]-2-[[2-(trifluoromethyl)cyclopropanecarbonyl]amino]benzamide;
(2R)—N-[4-chloro-2-[[(1S)-4,4-difluoro-1-[2-(methylamino)-2-oxo-acetyl]pentyl]carbamoyl]phenyl]spiro[2.2]pentane-2-carboxamide;
N-[(1S)-4,4-difluoro-1-[2-(methylamino)-2-oxo-acetyl]pentyl]-5-fluoro-2-[(3-fluorobenzoyl)amino]pyridine-3-carboxamide;
N-[(1S)-4,4-difluoro-1-[2-(methylamino)-2-oxo-acetyl]pentyl]-5-fluoro-2-[(3-fluorobicyclo[1.1.1]pentane-1-carbonyl)amino]pyridine-3-carboxamide;
5-chloro-N-[(1S)-4,4-difluoro-1-[2-(methylamino)-2-oxo-acetyl]pentyl]-2-[[1-(trifluoromethyl)cyclopropanecarbonyl]amino]pyridine-3-carboxamide;
N-[4-cyano-2-[[(1S)-4,4-difluoro-1-[2-(methylamino)-2-oxo-acetyl]pentyl]carbamoyl]phenyl]-3-(trifluoromethyl)bicyclo[1.1.1]pentane-1-carboxamide;
5-chloro-N-[(1S)-4,4-difluoro-1-[2-(methylamino)-2-oxo-acetyl]pentyl]-2-[(3-fluorobicyclo[1.1.1]pentane-1-carbonyl)amino]pyridine-3-carboxamide;
N-[(1S)-1-[2-(cyclopropylamino)-2-oxo-acetyl]-4,4-difluoro-pentyl]-5-(trifluoromethyl)-2-[[3-(trifluoromethyl)benzoyl]amino]pyridine-3-carboxamide;
N-[(1S)-1-[2-(cyclopropylamino)-2-oxo-acetyl]-4,4-difluoro-pentyl]-5-fluoro-2-[(3-fluorobenzoyl)amino]pyridine-3-carboxamide;
5-cyano-N-[(1S)-4,4-difluoro-1-[2-(methylamino)-2-oxo-acetyl]pentyl]-2-[(3-fluorobenzoyl)amino]benzamide;
N-[(1S)-4,4-difluoro-1-[2-(methylamino)-2-oxo-acetyl]pentyl]-5-fluoro-2-[[1-(trifluoromethyl)cyclopropanecarbonyl]amino]benzamide;
N-[(1S)-4,4-difluoro-1-[2-(methylamino)-2-oxo-acetyl]pentyl]-2-(2,2-dimethylpropanoylamino)-5-fluoro-pyridine-3-carboxamide;
5-chloro-N-[(1S)-1-[2-(cyclopropylamino)-2-oxo-acetyl]-4,4-difluoro-pentyl]-2-[[1-(trifluoromethyl)cyclopropanecarbonyl]amino]pyridine-3-carboxamide;
N-[(1S)-1-[2-(cyclopropylamino)-2-oxo-acetyl]-4,4-difluoro-pentyl]-5-methyl-2-[[3-(trifluoromethyl)benzoyl]amino]pyridine-3-carboxamide;
N-[(1S)-1-[2-(cyclopropylamino)-2-oxo-acetyl]-4,4-difluoro-pentyl]-2-(2,2-dimethylpropanoylamino)-5-fluoro-pyridine-3-carboxamide;
5-chloro-N-[(1S)-4,4-difluoro-1-[2-(methylamino)-2-oxo-acetyl]pentyl]-2-[[2-(trifluoromethyl)cyclopropanecarbonyl]amino]benzamide;
5-chloro-N-[(1S)-4,4-difluoro-1-[2-(methylamino)-2-oxo-acetyl]pentyl]-2-[[2-(trifluoromethyl)cyclopropanecarbonyl]amino]benzamide;
methyl N-[4-chloro-2-[[(1S)-4,4-difluoro-1-[2-(methylamino)-2-oxo-acetyl]pentyl]carbamoyl]phenyl]carbamate;
N-[4-chloro-2-[[(1S)-4,4-difluoro-1-[2-(methylamino)-2-oxo-acetyl]pentyl]carbamoyl]phenyl]-1-methyl-azetidine-3-carboxamide;
N-[4-chloro-2-[[(1S)-4,4-difluoro-1-[2-(methylamino)-2-oxo-acetyl]pentyl]carbamoyl]phenyl]-5-cyano-pyridine-3-carboxamide;
N-[4-chloro-2-[[(1S)-4,4-difluoro-1-[2-(methylamino)-2-oxo-acetyl]pentyl]carbamoyl]phenyl]pyridine-3-carboxamide;
5-chloro-N-[(1S)-4,4-difluoro-1-oxamoyl-pentyl]-2-(4,4,4-trifluorobutanoylamino)benzamide;
5-chloro-2-[(3,3-difluorocyclobutanecarbonyl)amino]-N-[(1S)-4,4-difluoro-1-[2-(methylamino)-2-oxo-acetyl]pentyl]pyridine-3-carboxamide;
methyl N-[6-chloro-4-[[(1S)-4,4-difluoro-1-[2-(methylamino)-2-oxo-acetyl]pentyl]carbamoyl]-3-pyridyl]carbamate;
methyl N-[5-chloro-3-[[(1S)-4,4-difluoro-1-[2-(methylamino)-2-oxo-acetyl]pentyl]carbamoyl]-2-pyridyl]carbamate;
5-chloro-N-[(1S)-4,4-difluoro-1-[2-(methylamino)-2-oxo-acetyl]pentyl]-2-(2-methylpropanoylamino)benzamide;
cyclopropyl N-[4-chloro-2-[[(1S)-4,4-difluoro-1-[2-(methylamino)-2-oxo-acetyl]pentyl]carbamoyl]phenyl]carbamate; and
5-chloro-N-[(1S)-4,4-difluoro-1-[2-(methylamino)-2-oxo-acetyl]pentyl]-2-[[(2S)-2-methoxypropanoyl]amino]benzamide.

In certain embodiments of the present invention the compound of formula I is selected form the group consisting of:

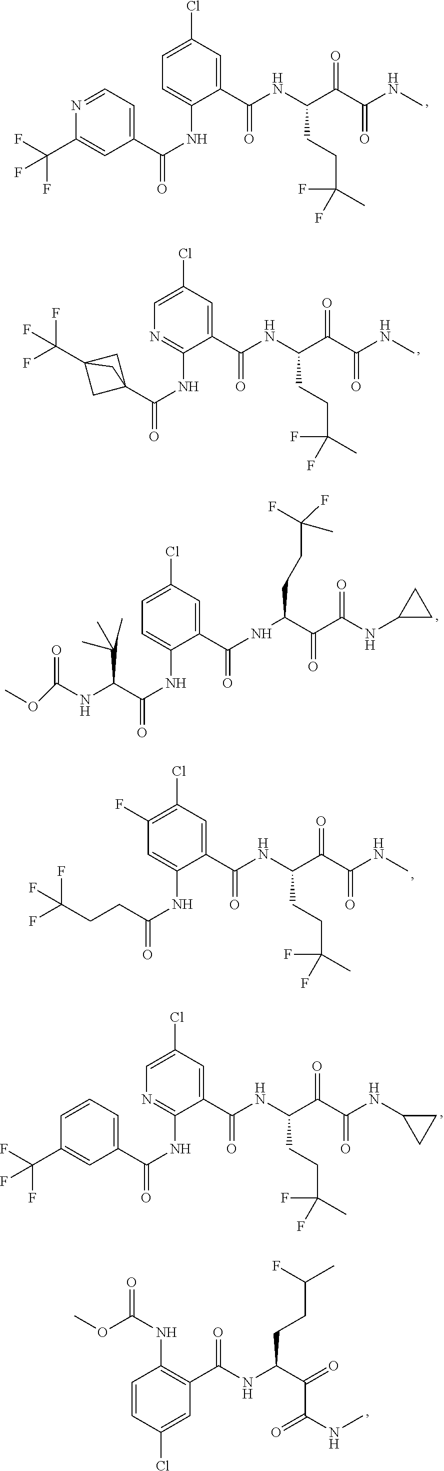

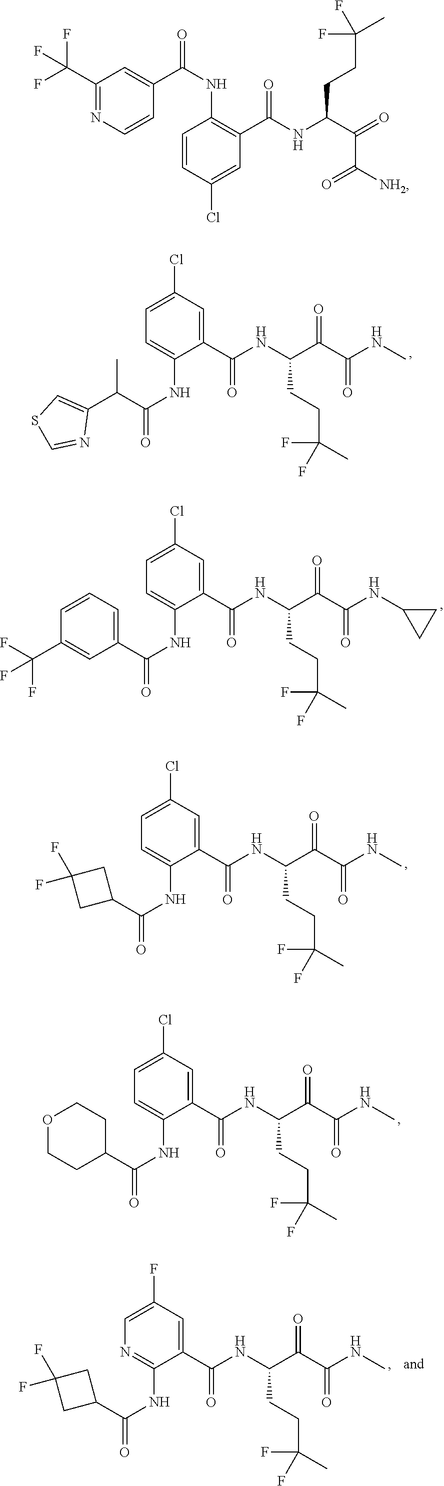 [SMILES: CC(F)(F)CC[C@H](NC(=O)c1cc(Cl)ccc1NC(=O)c1ccnc(C(F)(F)F)c1)C(=O)C(N)=O ; CNC(=O)C(=O)[C@H](CCC(C)(F)F)NC(=O)c1cc(Cl)ccc1NC(=O)C(C)c1cscn1 ; CC(F)(F)CC[C@H](NC(=O)c1cc(Cl)ccc1NC(=O)c1cccc(C(F)(F)F)c1)C(=O)C(=O)NC1CC1 ; CNC(=O)C(=O)[C@H](CCC(C)(F)F)NC(=O)c1cc(Cl)ccc1NC(=O)C1CC(F)(F)C1 ; CNC(=O)C(=O)[C@H](CCC(C)(F)F)NC(=O)c1cc(Cl)ccc1NC(=O)C1CCOCC1 ; CNC(=O)C(=O)[C@H](CCC(C)(F)F)NC(=O)c1cc(F)cnc1NC(=O)C1CC(F)(F)C1]

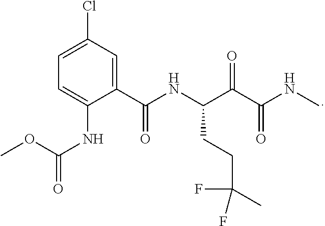

Also included within the scope of the present invention is a pharmaceutical composition which is comprised of a compound of Formula I as described above or a pharmaceutically acceptable salt thereof, and a pharmaceutically acceptable carrier. The pharmaceutical composition can be, for example, in the form of an orally administered tablet or capsule. The invention is also contemplated to encompass a pharmaceutical composition which is comprised of a pharmaceutically acceptable carrier and any of the compounds specifically disclosed in the present application, including pharmaceutically acceptable salts thereof. These and other aspects of the invention will be apparent from the teachings contained herein.

The invention also includes compositions for inhibiting protease in a coronavirus, treating a disease caused by a coronavirus, treating coronavirus infection and preventing coronavirus infection, in a mammal, comprising a compound of the invention in a pharmaceutically acceptable carrier. These compositions may optionally include other antiviral agents. The compositions can be added to blood, blood products, or mammalian organs in order to effect the desired inhibitions.

The invention further includes methods for prophylaxis or treatment of a coronavirus infection by administering compounds of formula I. Such coronavirus infections inculde a SARS-CoV, SARS-CoV-2 or MERS-CoV infection

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, ascorbate, adipate, alginate, aspirate, benzenesulfonate, benzoate, bicarbonate, bisulfate, bitartrate, borate, bromide, butyrate, camphorate, camphorsulfonate, camsylate, carbonate, chloride, clavulanate, citrate, cyclopentane propionate, diethylacetic, digluconate, dihydrochloride, dodecylsulfanate, edetate, edisylate, estolate, esylate, ethanesulfonate, formic, fumarate, gluceptate, glucoheptanoate, gluconate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, undeconate, valerate and the like. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Also included are the ammonium, calcium, magnesium, potassium, and sodium salts. Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethylamine, 2-diethylaminoethanol, 2-dimethylaminoethanol, ethanolamine, ethylamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Also, included are the basic nitrogen-containing groups that may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

These salts can be obtained by known methods, for example, by mixing a compound of the present invention with an equivalent amount and a solution containing a desired acid, base, or the like, and then collecting the desired salt by filtering the salt or distilling off the solvent. The compounds of the present invention and salts thereof may form solvates with a solvent such as water, ethanol, or glycerol. The compounds of the present invention may form an acid addition salt and a salt with a base at the same time according to the type of substituent of the side chain.

If the compounds of Formula I simultaneously contain acidic and basic groups in the molecule the invention also includes, in addition to the salt forms mentioned, inner salts or betaines (zwitterions).

The present invention encompasses all stereoisomeric forms of the compounds of Formula I. Unless a specific stereochemistry is indicated, the present invention is meant to comprehend all such isomeric forms of these compounds. Centers of asymmetry that are present in the compounds of Formula I can all independently of one another have (R) configuration or (S) configuration. When bonds to the chiral carbon are depicted as straight lines in the structural Formulas of the invention, it is understood that both the (R) and (S) configurations of the chiral carbon, and hence both each individual enantiomer and mixtures thereof, are embraced within the Formula. When a particular configuration is depicted, that enantiomer (either (R) or (S), at that center) is intended. Similarly, when a compound name is recited without a chiral designation for a chiral carbon, it is understood that both the (R) and (S) configurations of the chiral carbon, and hence individual enantiomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

Unless a specific enantiomer or diastereomer is indicated, the invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Where compounds of this invention are capable of tautomerization, all individual tautomers as well as mixtures thereof are included in the scope of this invention. The present invention includes all such isomers, as well as salts, solvates (including hydrates) and solvated salts of such racemates, enantiomers, diastereomers and tautomers and mixtures thereof.

In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the specifically and generically described compounds. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques well known to those skilled in the art or by processes analogous to those described in the general process schemes and examples herein using appropriate isotopically-enriched reagents and/or intermediates.

When any variable occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted” (with one or more substituents) should be understood as meaning that the group in question is either unsubstituted or may be substituted with one or more substituents.

Furthermore, compounds of the present invention may exist in amorphous form and/or one or more crystalline forms, and as such all amorphous and crystalline forms and mixtures thereof of the compounds of Formula I are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

Any pharmaceutically acceptable pro-drug modification of a compound of this invention which results in conversion in vivo to a compound within the scope of this invention is also within the scope of this invention. For example, esters can optionally be made by esterification of an available carboxylic acid group or by formation of an ester on an available hydroxy group in a compound. Similarly, labile amides can be made. Pharmaceutically acceptable esters or amides of the compounds of this invention may be prepared to act as pro-drugs which can be hydrolyzed back to an acid (or —COO— depending on the pH of the fluid or tissue where conversion takes place) or hydroxy form particularly in vivo and as such are encompassed within the scope of this invention. Examples of pharmaceutically acceptable pro-drug modifications include, but are not limited to, —C₁-C₆ alkyl esters and —C₁-C₆ substituted with phenyl esters.

Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof, and salts of pro-drug forms thereof, where such forms are possible unless specified otherwise.

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings:

A “subject” is a human or non-human mammal. In one embodiment, a subject is a human. In another embodiment, a subject is a primate. In another embodiment, a subject is a monkey. In another embodiment, a subject is a chimpanzee. In still another embodiment, a subject is a rhesus monkey.

As used herein, the terms “treatment” and “treating” refer to all processes in which there may be a slowing, interrupting, arresting, controlling, or stopping of the progression of a disease or disorder described herein. The terms do not necessarily indicate a total elimination of all disease or disorder symptoms.

The terms “preventing,” or “prophylaxis,” as used herein, refers to reducing the likelihood of contracting disease or disorder described herein, or reducing the severity of a disease or disorder described herein.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched. Unless otherwise indicated, an alkyl group is unsubstituted.

The term “fluoroalkyl,” as used herein refers to an alkyl group as defined above, wherein one or more of the alkyl group's hydrogen atoms has been replaced with a fluorine. In one embodiment, a fluoroalkyl group has from 1 to 6 carbon atoms. In another embodiment, a haloalkyl group is substituted with from 1 to 3 F atoms. Non-limiting examples of fluoroalkyl groups include —CH₂F, —CHF₂, —CF₃, and —CH₂CF₃. The term “C₁-C₆ fluoroalkyl” refers to a fluoroalkyl group having from 1 to 6 carbon atoms.

The term “halo,” as used herein, means —F, —Cl, —Br or —I.

The term “cycloalkyl” means a monocyclic or bicyclic saturated aliphatic hydrocarbon group having the specified number of carbon atoms. For example, “cycloalkyl” includes cyclopropyl, cyclobutyl, cyclopentyl, cyclohexyl, and so on. Bicyclic cycloalkyl ring systems include fused ring systems, where two rings share two atoms, spiro ring systems, where two rings share one atom, and bridged systems.

The term “aryl”, as used herein, represents a stable bicyclic or tricyclic ring system of up to 10 atoms in each ring, wherein at least one ring is aromatic, and all of the ring atoms are carbon. Bicyclic and tricyclic ring systems include fused ring systems, where two rings share two atoms, and spiro ring systems, where two rings share one atom.

The term “heteroaryl”, as used herein, represents a stable monocyclic or bicyclic ring system of up to 10 atoms in each ring, wherein at least one ring is aromatic, and at least one ring contains from 1 to 4 heteroatoms selected from the group consisting of O, N and S. Bicyclic heteroaryl ring systems include fused ring systems, where two rings share two atoms, and spiro ring systems, where two rings share one atom. Heteroaryl groups within the scope of this definition include but are not limited to: azaindolyl, benzoimidazolyl, benzisoxazolyl, benzofuranyl, benzofurazanyl, benzopyrazolyl, benzotriazolyl, benzothiophenyl, benzoxazolyl, carbazolyl, carbolinyl, cinnolinyl, dihydroindenyl, furanyl, indolinyl, indolyl, indolazinyl, indazolyl, isobenzofuranyl, isoindolyl, isoquinolyl, isothiazolyl, isoxazolyl, naphthalenyl, naphthpyridinyl, oxadiazolyl, oxazolyl, oxazoline, isoxazoline, pyranyl, pyrazinyl, pyrazolyl, pyrazolopyrimidinyl, pyridazinyl, pyridopyridinyl, pyridyl, pyrimidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, dihydrobenzodioxinyl, dihydropyrazoloxazinyl, dihydropyrazolyothiazinedioxidyl, methylenedioxy benzene, benzothiazolyl, benzothienyl, quinolinyl, isoquinolinyl, oxazolyl, tetra-hydroquinoline and 3-oxo-3,4dihydro-2N-benzo[b][1,4]thiazine. If the heteroaryl contains nitrogen atoms, it is understood that the corresponding N-oxides thereof are also encompassed by this definition.

The term “heterocycloalkyl”, “heterocycle,” or “heterocyclyl” as used herein is intended to mean a stable nonaromatic monocyclic or bicyclic ring system of up to 10 atoms in each ring, unless otherwise specified, containing from 1 to 4 heteroatoms selected from the group consisting of O, N, S, SO, or SO₂. In some embodiments, heterocycloalkyl are saturated. Bicyclic heterocyclic ring systems include fused ring systems, where two rings share two atoms, and spiro ring systems, where two rings share one atom. “Heterocycloalkyl” therefore includes, but is not limited to the following: azaspirononanyl, azaspirooctanyl, azetidinyl, dioxanyl, oxadiazaspirodecenyl, oxaspirooctanyl, oxazolidinonyl, piperazinyl, piperidinyl, pyrrolidinyl, morpholinyl, thiomorpholinyl, tetrahydrofuranyl, tetrahydropyranyl, dihydropiperidinyl, tetrahydrothiophenyl and the like. If the heterocycle contains a nitrogen, it is understood that the corresponding N-oxides thereof are also encompassed by this definition.

“Celite®” (Fluka) diatomite is diatomaceous earth and can be referred to as “celite”.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al., Protective Groups in Organic Synthesis (1991), Wiley, New York.

When any substituent or variable (e.g., R₂) occurs more than one time in any constituent or in Formula I, its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts.

The invention also relates to medicaments containing at least one compound of the Formula I and/or of a pharmaceutically acceptable salt of the compound of the Formula I and/or an optionally stereoisomeric form of the compound of the Formula I or a pharmaceutically acceptable salt of the stereoisomeric form of the compound of Formula I, together with a pharmaceutically suitable and pharmaceutically acceptable vehicle, additive and/or other active substances and auxiliaries.

The term “patient” used herein is taken to mean mammals such as primates, humans, sheep, horses, cattle, pigs, dogs, cats, rats, and mice.

The term “coronavirus” includes HCoV-229E, HCoV-NL63, HCoV-OC43, HCoV-HKU1, severe acute respiratory syndrome coronavirus (SARS-CoV), Middle East respiratory syndrome coronavirus (MERS-CoV) and SARS-CoV-2.

The medicaments according to the invention can be administered by oral, inhalative, rectal or transdermal administration or by subcutaneous, intraarticular, intraperitoneal or intravenous injection. Oral administration is preferred. Coating of stents with compounds of the Formula (I) and other surfaces which come into contact with blood in the body is possible.

The invention also relates to a process for the production of a medicament, which comprises bringing at least one compound of the Formula (I) into a suitable administration form using a pharmaceutically suitable and pharmaceutically acceptable carrier and optionally further suitable active substances, additives or auxiliaries.

Suitable solid or galenical preparation forms are, for example, granules, powders, coated tablets, tablets, (micro)capsules, suppositories, syrups, juices, suspensions, emulsions, drops or injectable solutions and preparations having prolonged release of active substance, in whose preparation customary excipients such as vehicles, disintegrants, binders, coating agents, swelling agents, glidants or lubricants, flavorings, sweeteners and solubilizers are used. Frequently used auxiliaries which may be mentioned are magnesium carbonate, titanium dioxide, lactose, mannitol and other sugars, talc, lactose, gelatin, starch, cellulose and its derivatives, animal and plant oils such as cod liver oil, sunflower, peanut or sesame oil, polyethylene glycol and solvents such as, for example, sterile water and mono- or polyhydric alcohols such as glycerol.

The dosage regimen utilizing the protease inhibitors of the instant invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the protease inhibitors, when used for the indicated effects, will range between about 0.01 mg per kg of body weight per day (mg/kg/day) to about 30 mg/kg/day, for instance, 0.01-20 mg/kg/day, 0.01-15 mg/kg/day, 0.01-10 mg/kg/day or 0.01-5 mg/kg/day (unless specified otherwise, amounts of active ingredients are on free base basis). For example, an 80 kg patient would receive between about 0.8 mg/day and 2.4 g/day, e.g., 0.8-1600 mg/day, 0.8-1200 mg/day, 0.8-800 mg/kg/day, or 0.8-400 mg/day. A suitably prepared medicament for once a day administration would thus contain between 0.8 mg and 2.4 g, between 0.8 mg and 1600 mg, between 0.8 mg and 1200 mg, between 0.8 mg and 800 mg, or between 0.8 and 400 mg, e.g., 1 mg, 4 mg, 8 mg, 10 mg, 20 mg, 40 mg, 80 mg, 160 mg, 200 mg, 300 mg, or 400 mg. Advantageously, the protease inhibitors may be administered in divided doses of two, three, or four times daily. For administration twice a day, a suitably prepared medicament would contain between 0.4 mg and 1.2 g, between 0.4 mg and 800 mg, between 0.4 mg and 600 mg, between 0.4 mg and 400 mg, or between 0.4 and 200 mg, e.g., 0.5 mg, 2 mg, 4 mg, 5 mg, 10 mg, 20 mg, 40 mg, 80 mg, 100 mg, 150 mg, or 200 mg.

Intravenously, the patient would receive the active ingredient in quantities sufficient to deliver between 0.01-15 mg/kg/day, e.g., 0.01-7.5 mg/kg/day or 0.1-5 mg/kg/day. Such quantities may be administered in a number of suitable ways, e.g., large volumes of low concentrations of active ingredient during one extended period of time or several times a day, low volumes of high concentrations of active ingredient during a short period of time, e.g., once a day. Glucuronic acid, L-lactic acid, acetic acid, citric acid or any pharmaceutically acceptable acid/conjugate base with reasonable buffering capacity in the pH range acceptable for intravenous administration may be used as buffers. The choice of appropriate buffer and pH of a formulation, depending on solubility of the drug to be administered, is readily determined by a person having ordinary skill in the art.

Compounds of Formula I can be administered both as a monotherapy and in combination with additional therapeutic agents (also referred to herein as “second therapeutic agents”), including other antivirals or treatments of coronavirus infection.

The protease inhibitors of the instant invention can also be co-administered with suitable antivirals, including, but not limited to, agents that inhibit the replication of viruses such as nucleoside polymerase inhibitors, agents that induce viral error catastrophe protease inhibitors, eEF1A inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors. In a class of the invention, the protease inhibitors of the instant invention can be co-administered with a nucleoside polymerase inhibitor, a protease inhibitor, or a combination thereof. Skilled practitioners will acknowledge that such antivirals in some cases may be co-administered as prodrugs.

Polymerase inhibitors that can be co-administered with the protease inhibitors of the instant invention include, but are not limited to, clevudine, remdesivir (VEKLURY), favipiravir (AVIGAN) and AT-527.

Protease inhibitors that can be co-administered with the protease inhibitors of the instant invention include, but are not limited to, camostat mesylate, upamostat, SLV213, PF-0083523, CDI-45205, ALG-097111, GC-376 and TJC-0642.

Agents that induce viral error catastrophe that can be co-administered with the protease inhibitors of the invention include molnupiravir and nirmatrelvir.

eEF1A inhibitors that can be co-administered with the protease inhibitors of the instant invention include, but are not limited to, plitidepsin.

Androgen receptor antagonists that can be co-administered with the protease inhibitors of the instant invention include, but are not limited to, proxalutamide.

Dihydroorotate dehydrogenase (DHODH) inhibitors that can be co-administered with the protease inhibitors of the instant invention include, but are not limited to, PTC299 and brequinlar.

Sphingosine kinase inhibitors that can be co-administered with the protease inhibitors of the instant invention include, but are not limited to, opaganib.

MEK inhibitors that can be co-administered with the protease inhibitors of the instant invention include, but are not limited to, ATR-002.

Antimalarials that can be co-administered with the protease inhibitors of the instant invention include, but are not limited to, tafenoquine (ARAKODA).

CCR5 inhibitors that can be co-administered with the protease inhibitors of the instant invention include, but are not limited to, maraviroc and vicriviroc.

PIKfyve kinase inhibitors that can be co-administered with the protease inhibitors of the instant invention include, but are not limited to, Apilimod.

Serine protease inhibitors that can be co-administered with the protease inhibitors of the instant invention include, but are not limited to, nafamostat mesylate.

Glycosylation inhibitors that can be co-administered with the protease inhibitors of the instant invention include, but are not limited to, WP1122.

Alternatively or additionally, one or more additional pharmacologically active agents may be administered in combination with a compound of the invention. The additional active agent (or agents) is intended to mean a pharmaceutically active agent (or agents) that is active in the body, including pro-drugs that convert to pharmaceutically active form after administration, which is different from the compound of the invention, and also includes free-acid, free-base and pharmaceutically acceptable salts of said additional active agents when such forms are sold commercially or are otherwise chemically possible. Generally, any suitable additional active agent or agents, including but not limited to polymerase nucleoside inhibitors, protease inhibitors, agents that induce viral error catastrophe, eEF1A inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors can be used in any combination with the compound of the invention in a single dosage formulation (a fixed dose drug combination), or may be administered to the patient in one or more separate dosage formulations which allows for concurrent or sequential administration of the active agents (co-administration of the separate active agents).

Typical doses of the protease inhibitors of the invention in combination with other suitable polymerase nucleoside inhibitors, protease inhibitors, agents that induce viral error catastrophe, eEF1A inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors may be the same as those doses of the protease inhibitors administered without coadministration of additional polymerase nucleoside inhibitors, protease inhibitors, agents that induce viral error catastrophe, eEF1A inhibitors, androgen receptor antagonists, Dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors, or may be substantially less that those doses of protease inhibitors administered without coadministration of polymerase nucleoside inhibitors, protease inhibitors, agents that induce viral catastrophe, eEF1A inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors depending on a patient's therapeutic needs.

The compounds are administered to a mammal in a therapeutically effective amount. By “therapeutically effective amount” it is meant an amount of a compound of the present invention that, when administered alone or in combination with an additional therapeutic agent to a mammal, is effective to treat (i.e., prevent, inhibit or ameliorate) the viral condition or treat the progression of the disease in a host.

The compounds of the invention are preferably administered alone to a mammal in a therapeutically effective amount. However, the compounds of the invention can also be administered in combination with an additional therapeutic agent, as defined below, to a mammal in a therapeutically effective amount. When administered in a combination, the combination of compounds is preferably, but not necessarily, a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of each of the compounds when administered individually as a single agent. In general, a synergistic effect is most clearly demonstrated at suboptimal concentrations of the compounds. Synergy can be in terms of lower cytotoxicity, increased anticoagulant effect, or some other beneficial effect of the combination compared with the individual components.

By “administered in combination” or “combination therapy” it is meant that the compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

The present invention is not limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the relevant art and are intended to fall within the scope of the appended claims.

### General Procedures

Starting materials and intermediates were purchased or were prepared using known procedures described in the chemical synthetic literature or as otherwise described. The preparation of the various starting materials used herein is well within the skill of a person versed in the art. Routes applied to the synthesis of compounds of Formula I are described in the following schemes. In some cases, the sequence of reaction steps may be varied to facilitate reactions or to avoid unwanted reaction products. In some cases, the final product may be further modified, for example, by manipulation of substituents. These manipulations may include, but are not limited to, reduction, oxidation, alkylation, acylation, and hydrolysis reactions which are commonly known to those skilled in the art. Because the schemes are an illustration, the invention should not be construed as being limited by the chemical reactions and conditions expressed. The examples described below are provided so that the invention might be more fully understood. These examples are illustrative only and should not be construed as limiting the invention in any way

It is understood that a chiral center in a compound may exist in the S or R absolute configuration, or as a mixture of both. Within a molecule, each bond drawn as a straight line from a chiral center includes both the R and S stereoisomers as well as mixtures thereof. An asterisk denotes a stereocenter in a single configuration, either R or S. Absolute stereochemistry of separate stereoisomers in the examples and intermediates are not determined unless stated otherwise in an example or explicitly in the nomenclature.

Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are carried out by employing reactions as shown in the schemes and examples herein, in addition to other standard manipulations such as ester hydrolysis, cleavage of protecting groups, etc., as may be known in the literature or exemplified in the experimental procedures.

Reactions sensitive to moisture or air were performed under nitrogen or argon using anhydrous solvents and reagents. The progress of reactions was determined by either liquid chromatography-mass spectrometry (LCMS) or analytical thin layer chromatography (TLC) usually performed with Merck KGaA glass-backed TLC plates, silica gel 60 F₂₅₄.

Analytical LCMS was commonly performed on a Waters SQD single quadrupole mass spectrometer with electrospray ionization in positive ion detection mode (mass range set at 150-900 daltons, data collected in centroid mode and scan time set to 0.2 seconds) and a Waters Acquity UPLC system (binary solvent manager, sample manager, and TUV). The column used was a Waters Acquity BEH C18 1×50 mm, 1.7 μm, heated to 50° C. The mobile phases used were modified with either acidic or basic additives. The acidic mobile phase consisted of 0.1% trifluoroacetic acid in water for Solvent A and 100% acetonitrile for Solvent B. A two-minute run was established at a flow rate of 0.3 ml/min with Initial conditions of 95% Solvent A and ramping up to 99% Solvent B at 1.60 minutes and holding at 99% Solvent B for 0.40 minutes. The injection volume was 0.5 μL using partial loop needle overfill injection mode. The TUV monitored wavelength 215 or 254 nm with a sampling rate of 20 points/second, normal filter constant and absorbance data mode. The basic mobile phase consisted of 0.1% ammonium hydroxide in water for solvent A and 100% Acetonitrile for solvent B. A two-minute run was established at a flow rate of 0.3 ml/min with initial conditions of 99% Solvent A and ramping up to 99% Solvent B at 1.90 minutes and holding at 99% Solvent B for 0.10 minutes. A five-minute run was established at a flow rate of 0.3 ml/min with initial conditions of 95% Solvent A and ramping up to 99% Solvent B at 4.90 minutes and holding at 99% Solvent B for 0.10 minutes. For both methods, the injection volume was 5.0 μL using Partial Loop Needle Overfill Injection mode. The TUV monitored wavelength 215 nm with a sampling rate of 20 points/second, normal filter constant and absorbance data mode. Alternatively, a commonly used system consisted of a Waters ZQ™ platform with electrospray ionization in positive ion detection mode with an Agilent 1100 series HPLC with autosampler. The column was commonly a Waters Xterra MS C18, 3.0×50 mm, 5 μm or a Waters Acquity UPLC® BEH C18 1.0×50 mm, 1.7 μm. The flow rate was 1 mL/min, and the injection volume was 10 μL. UV detection was in the range 210-400 nm. The mobile phase consisted of solvent A (water plus 0.05% TFA) and solvent B (MeCN plus 0.05% TFA) with a gradient of 100% solvent A for 0.7 min changing to 100% solvent B over 3.75 min, maintained for 1.1 min, then reverting to 100% solvent A over 0.2 min.

Preparative reverse-phase chromatography was generally carried out on a Teledyne ISCO ACCQPrep HP125 or HP150 apparatus equipped with UV and ELSD detectors. The UV detector typically monitored wavelengths of 215 and 254 nm. The column was commonly one of the following: Waters XBridge Prep C18 OBD 5 μm 30×150 mm, Waters XBridge Prep C18 OBD 5 μm 30×250 mm, Waters XBridge Prep C18 OBD 5 μm 50×250 mm, Waters SunFire Prep C18 OBD 5 μm 30×150 mm, Waters SunFire Prep C18 OBD 10 μm 30×150 mm, Waters SunFire Prep C18 OBD 5 μm 50×250 mm, Waters SunFire Prep C18 OBD 10 μm 50× 250 mm, or Phenomenex Luna Prep C18 5 μm 50×250 mm. The mobile phases consisted of mixtures of 0.1% TFA in acetonitrile with 0.1% TFA in water or mixtures of 100% acetonitrile with 5 mM (NH₄)HCO₃. Alternatively, a commonly used system was a Waters Chromatography Workstation configured with an LCMS system consisting of: Waters ZQ™ single quad MS system with Electrospray Ionization, Waters 2525 Gradient Pump, Waters 2767 Injector/Collector, Waters 996 PDA Detector. MS conditions were: 150-750 amu, positive electrospray, collection triggered by MS. Columns used were commonly a Waters SunFire C18 5 μm 30×150 mm, a Boston Green ODS 5 μm 150×30 mm, or a YMC-Actus Triart C18 5 μm 150×30 mm column. The mobile phases consisted of mixtures of acetonitrile (10-100%) in water containing 0.1% TFA. Flow rates were maintained at 50 mL/min, and the UV detection range was 210-400 nm. An additional preparative HPLC system used was a Gilson Workstation consisting of: Gilson GX-281 Injector/Collector, Gilson UV/VIS-155 Detector, Gilson 333 and 334 Pumps, and either a Phenomenex Gemini-NX C18 5 μm 50×250 mm column, a Waters XBridge Prep C18 OBD 5 μm 30×250 mm, or a Welch Xtimate C18 5 μm 150×25 mm. The mobile phases consisted of mixtures of acetonitrile (0-75%) in water containing 5 mM (NH₄)HCO₃. Flow rates were maintained at 50 mL/min for the Waters XBridge column, 90 mL/min for the Phenomenex Gemini column, and 25 mL/min for the Welch Xtimate column. The UV detection range was 210-400 nm. Mobile phase gradients were optimized for the individual compounds.

Flash chromatography was usually performed using an ISCO CombiFlash Rf apparatus, a Biotage® Flash Chromatography apparatus (Dyax Corp.), or an ISCO CombiFlash® Companion XL apparatus on silica gel (60 Å pore size) in pre-packed RediSep Rf, RediSep Rf Gold, or SepaFlash columns. Mobile phases generally consisted of mixtures of hexanes or dichloromethane with EtOAc, 3:1 EtOAc: EtOH, or MeOH. Mobile phase gradients were optimized for the individual compounds.

Chiral chromatography was commonly performed by supercritical fluid chromatography with a column chosen from one of the following: Daicel CHIRALPAK AD-H 2×25 cm, Daicel CHIRALPAK AD-H 3×25 cm, YMC Chiral ART Cellulose-SC, Lux Cellulose-2 5 μm 30×250 mm, or Exsil Chiral-NR 8 μm 30×250 mm. Mobile phases consisted of mixtures of CO₂ with methanol, ethanol, isopropanol+0.1% diethylamine, isopropanol+0.1% NH₄OH, or 1:1 isopropanol: hexanes+0.1% 2 M NH₃/MeOH. Mobile phase gradients were optimized for the individual compounds. Pressure was typically maintained at 100 bar, and flow rates ranged from 50-200 mL/min. UV monitoring was generally carried out at 220 or 205 nM.

¹H NMR data were typically acquired using using a Bruker NEO 500 MHz NMR spectrometer equipped with a room temperature 5 mm BBF iProbe, a Bruker Avance NEO 400 MHz NMR spectrometer equipped with a Bruker PI HR-BBO400S1-BBF/H/D-5.0-Z SP probe, or a Bruker Avance III 500 MHz NMR spectrometer equipped with a Bruker 5 mm PABBO probe. Chemical shift values are reported in delta (δ) units, parts per million (ppm). Chemical shifts for ¹H NMR spectra are given relative to signals for residual non-deuterated solvent (CDCl₃ referenced at δ 7.26 ppm; DMSO-d₆ referenced at δ 2.50 ppm and CD₃OD referenced at δ 3.31 ppm). Multiplets are reported by the following abbreviations: s=singlet, d=doublet, t=triplet, q=quartet, dd=doublet of doublets, m=multiplet or overlap of nonequivalent resonances. Coupling constants (J) are reported in Hertz (Hz). When compounds appear as mixtures of rotamers by NMR, spectral data corresponding to the major species observed in solution are reported.

### ABBREVIATIONS

ACN is acetonitrile; AOP is tris(dimethylamino)(3H-1,2,3-triazolo[4,5-b]pyridin-3-yloxy)phosphorus hexafluorophosphate; aq. is aqueous; Bn is benzyl; Boc is tert-butoxycarbonyl; Burgess Reagent is methyl N-(triethylammoniumsulfonyl)carbamate; Cbz is benzyloxycarbonyl; CDI is 1,1′-carbonyldiimidazole; DCM is dichloromethane; DIBAL or DIBAL-H is diisobutylaluminium hydride; DIEA or DIPEA is N,N-diisopropylethylamine; DMA is dimethylacetamide, DMF is N,N-dimethylformamide; DMP is Dess-Martin periodinane; DMSO is dimethyl sulfoxide; EDC or EDCI is 1-ethyl-3-(3-dimethylaminopropyl)carbodiimide; ELSD is evaporative light scattering detector; Et is ethyl; EtOAc is ethyl acetate; HATU is (1-[Bis(dimethylamino)methylene]-1H-1,2,3-triazolo[4,5-b]pyridinium 3-oxide hexafluorophosphate; HOBt is hydroxybenzotriazole; HPLC is high-pressure liquid chromatography; LAH is lithium aluminum hydride; LCMS is liquid chromatography-mass spectrometry; LiHMDS or LHMDS=Lithium bis(trimethylsilyl)amide; LRMS is low resolution mass spectrometry; mCPBA is meta-chloroperoxybenzoic acid; Me is methyl; MeCN is acetonitrile; MeOH is methanol; MTBE is methyl tert-butyl ether; OAc is acetate; OMe is methoxy; Na2CO3 is sodium carbonate; NaHCO₃ is sodium bicarbonate; Na₂SO₄ is anhydrous sodium sulfate; NH₄HCO₃ is ammonium bicarbonate; NaOH is sodium hydroxide; NMP is N-methyl-2-pyrrolidone; NMR is nuclear magnetic resonance; Pd is Palladium; Pd/C is palladium on carbon; PE is petroleum ether; Ph is phenyl; POCl₃ is phosphorus oxychloride; Rochelle's salt is Sodium Potassium Tartrate; RP HPLC is reverse phase high pressure liquid chromatography; RT or rt is room temperature; sat. is saturated; SFC is supercritical fluid chromatography; TEMPO is (2,2,6,6-tetramethylpiperidin-1-yl)oxy; ^{t}Bu is tert-butyl; TBS is tert-butyldimethylsilyl; TEA is triethylamine; TFA is trifluoroacetic acid; THF is tetrahydrofuran; TMS is trimethylsilyl; UV is ultraviolet.

As illustrated in Scheme A, in general, compounds of the invention can be prepared by acylation of an appropriately functionalized amine A-1 to provide compounds of formula A-2. Esters A-2 can be hydrolyzed to yield acids of formula A-3, which can be coupled with amines of formula INT-1 to afford products of formula A-4. Hydroxyamides A-4 can be oxidized to afford ketoamides of formula A-5. In some embodiments, stereoisomers may be separated during the course of the synthesis. Amines of type A-1, acylating agents, and amines of type INT-1 are commercially available or may be synthesized from appropriate intermediates.

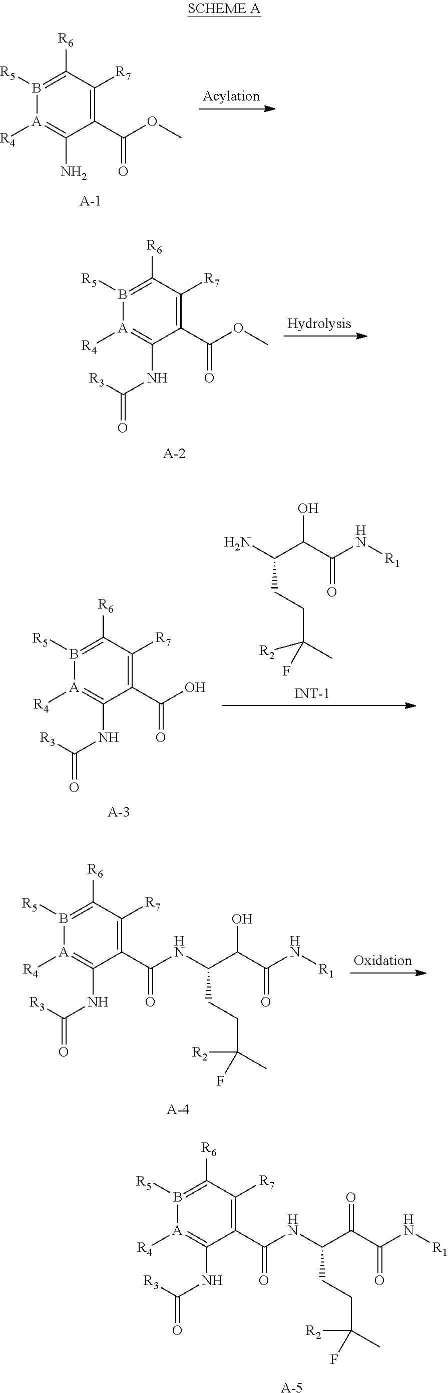

As illustrated in Scheme B, in general, compounds of the invention can be prepared by acylation of an appropriately functionalized amine B-1 to provide compounds of formula B-2, which can be coupled with amines of formula INT-1 to afford products of formula B-3. Hydroxyamides B-3 can be oxidized to afford ketoamides of formula B-4. In some embodiments, stereoisomers may be separated during the course of the synthesis. Amines of type B-1, acylating agents, and amines of type INT-1 are commercially available or may be synthesized from appropriate intermediates.

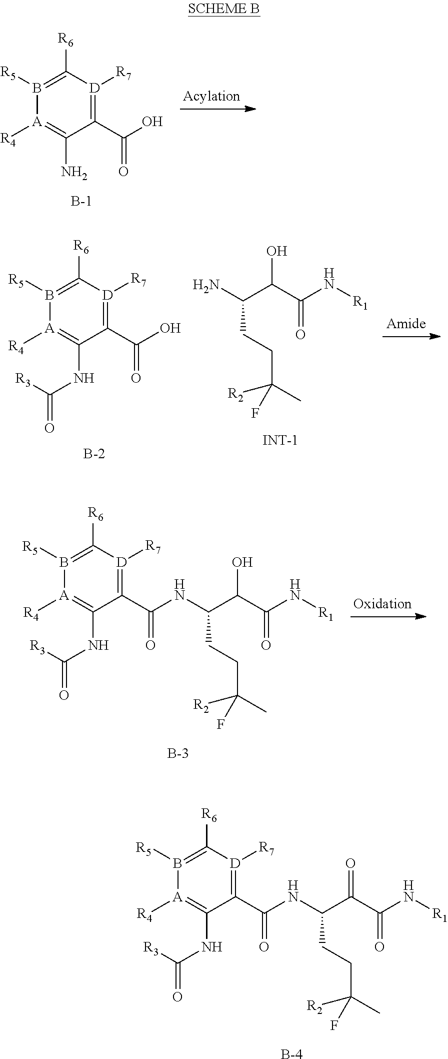

As illustrated in Scheme C, in general, compounds of the invention can be prepared by amidation of an appropriate aryl-halide/heteroaryl-halide C-1 (X=Cl or Br) with appropriate primary amide coupling partner to provide compounds of formula C-2. Esters C-2 can be hydrolyzed to yield acids of formula C-3 which can be coupled with amines of formula INT-1 to afford products of formula C-4. Hydroxyamides C-4 can be oxidized to afford ketoamides of formula C-5. In some embodiments, stereoisomers may be separated during the course of the synthesis. Aryl-halides/heteroaryl-halides of type C-1, primary amide coupling partners, and amines of type INT-1 are commercially available or may be synthesized from appropriate intermediates.

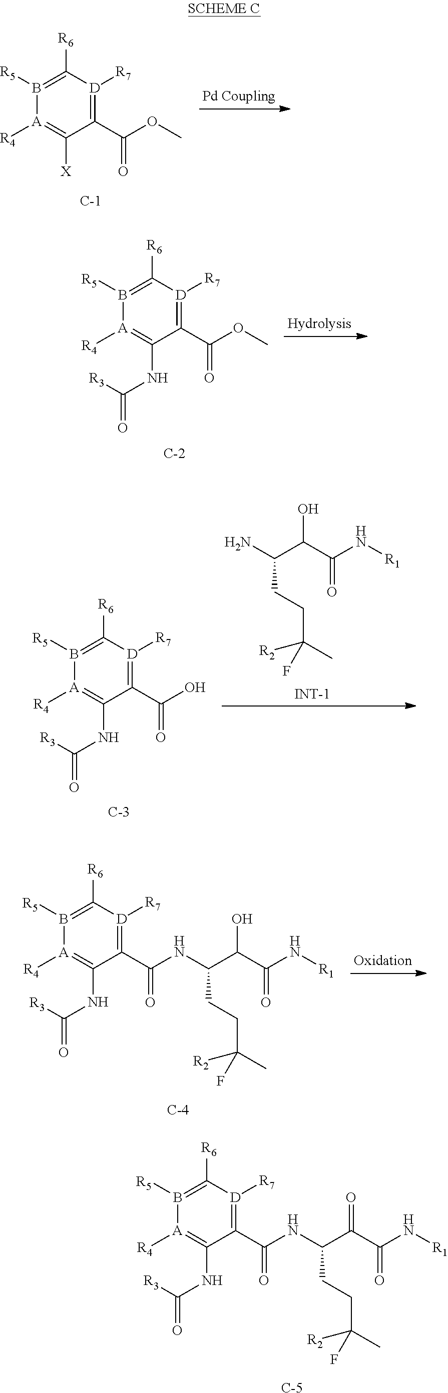

### Synthesis of Intermediates

In the section below, the preparation of certain intermediates useful in preparing the compounds of the invention are described.

### Intermediate 1

### (2S,3S)-3-(Benzyl((S)-1-phenylethyl)amino)-6,6-difluoro-2-hydroxyheptanoic acid

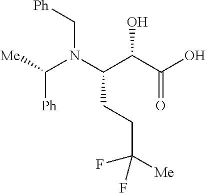 [SMILES: C[C@@H](c1ccccc1)N(Cc1ccccc1)[C@@H](CCC(C)(F)F)[C@H](O)C(=O)O]

Step 1: Methyl 4,4-difluoropentanoate

To a stirred solution of methyl 4-oxopentanoate (710 g, 5.46 mol, 1.00 equiv) in DCM (7100 mL) was added DAST (2640 g, 16.4 mol, 3.00 equiv) dropwise at 10° C. under nitrogen atmosphere. The mixture was stirred at RT for 72 h. The reaction was quenched by the addition of NaHCO₃ (7000 mL) at 0° C. The resulting mixture was extracted with CH₂Cl₂ (2×1500 mL). The combined organic layers were washed with NaHCO₃ (2×5000 mL), brine (1×5000 mL), dried over anhydrous Na₂SO₄, filtered, and the filtrate was directly used in next step.

### Step 2: 4,4-Difluoropentanal

To the solution from the previous step was added DIBAL (1 M in DCM, 6550 mL, 6550 mmol, 1.20 equiv) dropwise at −78° C. under nitrogen atmosphere. The resulting mixture was stirred for 2 h at −78° C. The mixture was acidified to pH 2-3 with HCl (2 M). The resulting mixture was extracted with CH₂Cl₂ (2×1000 mL), and dried over anhydrous Na₂SO₄. After filtration, the filtrate was directly used in next step.

Step 3: tert-Butyl (E)-6,6-difluorohept-2-enoate

To the solution from the previous step was added tert-butyl 2-(triphenyl-λ⁵ -phosphanylidene)acetate (2050 g, 5450 mmol, 1.00 equiv.), then stirred overnight at room temperature under nitrogen atmosphere. The resulting mixture was concentrated under vacuum. The residue was purified by silica gel column chromatography, eluting with 0-5% ethyl acetate in petroleum ether to afford the title compound.

Step 4: tert-Butyl(2S,3S)-3-(benzyl((S)-1-phenylethyl)amino)-6,6-difluoro-2-hydroxyheptanoate

To a stirred solution of benzyl[(1S)-1-phenylethyl]amine (730 g, 3450 mmol, 1.20 equiv.) in THF (6340 mL) was added n-hexyllithium (1700 mL, 3740 mmol, 1.30 equiv.) dropwise at −60° C. under nitrogen atmosphere. The resulting mixture was stirred for 30 min at −60° C. To the stirred solution was added tert-butyl (2E)-6,6-difluorohept-2-enoate (634 g, 2880 mmol, 1.00 equiv.) in THF (634 mL) dropwise at −60° C. The resulting mixture was stirred for 30 min at −60° C. Then to the solution was added (1S)-7,7-dimethyl-1-[(oxaziridine-2-sulfonyl)methyl]bicyclo[2.2.1]heptan-2-one (1050 g, 4030 mmol, 1.40 equiv.) in portions at −60° C. The resulting mixture was stirred for 2 h at −60° C. The reaction was quenched with AcOH (311 g, 5180 mmol, 1.80 equiv.) at −60° C. The mixture was basified to pH 8 with aqueous NaHCO₃. The resulting mixture was extracted with EtOAc (2×3000 mL). The combined organic layers were washed with brine (1×5000 mL) and dried over anhydrous Na₂SO₄. After filtration, the filtrate was concentrated under reduced pressure. The residue was purified by trituration with MTBE (5000 mL). The resulting mixture was filtered and the filtrate was concentrated under reduced pressure. The residue was purified by silica gel column chromatography eluting with 0-10% ethyl acetate in petroleum ether to give the title compound. LRMS m/z: (M+H)⁺ calculated 448.3; found 448.3.

Step 5: (2S,3S)-3-(Benzyl((S)-1-phenylethyl)amino)-6,6-difluoro-2-hydroxyheptanoic acid

A solution of tert-butyl (2S,3S)-3-{benzyl[(1S)-1-phenylethyl]amino}-6,6-difluoro-2-hydroxyheptanoate (980 g, 2190 mmol, 1.00 equiv.) in toluene (4900 mL) was treated with TFA (1500 g, 13100 mmol, 6.00 equiv.) for 5 h at 50° C. under nitrogen atmosphere. The resulting mixture was concentrated under vacuum. The residue was purified by silica gel column chromatography eluting with 0-45% ethyl acetate in petroleum ether to give the title compound. LRMS m/z: (M+H)⁺ calculated 392.2; found 392.2.

### Intermediate 2

### (2S,3S)-3-amino-6,6-difluoro-2-hydroxy-N-methylheptanamide, HCl

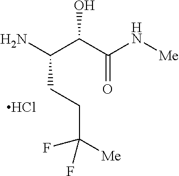 [SMILES: Cl ; CNC(=O)[C@@H](O)[C@@H](N)CCC(C)(F)F]

Step 1: (2S,3S)-3-(benzyl((S)-1-phenylethyl)amino)-6,6-difluoro-2-hydroxy-N-methylheptanamide

A stirred solution of (2S,3S)-3-(benzyl((S)-1-phenylethyl)amino)-6,6-difluoro-2-hydroxyheptanoic acid (45 g, 115 mmol), methylamine HCl (15.5 g, 230 mmol) and CH₂Cl₂ (460 mL) was treated with NMM (50.6 ml, 460 mmol) followed by HATU (54.6 g, 144 mmol). The mixture was stirred at ambient temperature overnight. The mixture was diluted with saturated aqueous NaHCO₃ and then extracted with CH₂Cl₂. The organic portion was washed with brine and dried (MgSO₄), and then the solvent was removed under reduced pressure. The residue was dissolved in 300 mL 1 N HCl and then washed with 500 mL of Et₂O. The aqueous portion was separated, basified with 1 N NaOH and then extracted with EtOAc. The combined organic portions were washed with brine, dried (MgSO₄), and filtered. The solvent was removed under reduced pressure to give the title compound. LRMS m/z: (M+H)⁺ calculated 406.2; found 406.4.

Step 2: (2S,3S)-3-amino-6,6-difluoro-2-hydroxy-N-methylheptanamide hydrochloride

A solution of (2S,3S)-3-(benzyl((S)-1-phenylethyl)amino)-6,6-difluoro-2-hydroxy-N-methylheptanamide (7.5 g, 18.54 mmol) in EtOH (185 mL) was treated with acetic acid (3.18 mL, 55.6 mmol) and 10 wt % Pd/C (1.12 g, 0.927 mmol) and then stirred under 1 atm H₂ for 18 hours. The mixture was filtered through celite and the celite pad was washed with EtOH. The solvent was removed under reduced pressure and then the residue was treated with 50 mL MeOH followed by 20 mL 4 N HCl in dioxane. The mixture was stirred for 5 minutes and then the solvent was removed under reduced pressure to give title compound. LRMS m/z: (M+H)⁺ calculated 211.2; found 211.2.

### Intermediate 3

The following compound was prepared in essentially the same manner as above:

| Intermediate # | Structure | Name | LRMS m/z (M + H)⁺ |
|---|---|---|---|
| 3 | 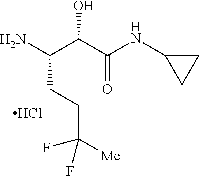 [SMILES: Cl ; CC(F)(F)CC[C@H](N)[C@H](O)C(=O)NC1CC1] | (2S,3S)-3-amino-N- cyclopropyl-6,6-difluoro- 2-hydroxyheptanamide hydrochloride | Calculated: 237.1 Found: 237.2 |

### Intermediate 4

### (S)-5,5-difluoro-1,1-dimethoxyhexan-2-amine

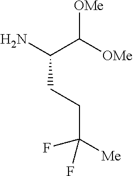 [SMILES: COC(OC)[C@@H](N)CCC(C)(F)F]

Step 1:3-(tert-butyl) 4-methyl (R)-2,2-dimethyloxazolidine-3,4-dicarboxylate

To a stirred solution of methyl (2R)-2-[(tert-butoxycarbonyl)amino]-3-hydroxypropanoate (800 g, 3650 mmol, 1.00 equiv) and 2,2-dimethoxypropane (3800 g, 36490 mmol, 10.00 equiv) in acetone (8 L) was added BF₃·Et₂O (25.9 g, 182 mmol, 0.05 equiv) dropwise at room temperature. The resulting mixture was stirred for 3 h at room temperature. The resulting mixture was concentrated under reduced pressure. The residue was purified by silica gel column chromatography eluting with petroleum ether:ethyl acetate (10:1) to afford the title compound. LRMS m/z: (M+H)⁺ calculated 260.2; found 260.2.

Step 2: tert-Butyl(S)-4-(hydroxymethyl)-2,2-dimethyloxazolidine-3-carboxylate

To a stirred solution of 3-tert-butyl 4-methyl (4R)-2,2-dimethyl-1,3-oxazolidine-3,4-dicarboxylate (800 g, 3090 mmol, 1.00 equiv) in THF (6.4 L) was added LiAlH₄ (234 g, 6170 mmol, 2.00 equiv) dropwise at 0° C. under nitrogen atmosphere. The resulting mixture was stirred for overnight at room temperature under nitrogen atmosphere. The reaction was quenched with water/ice at 0° C. The resulting mixture was concentrated under reduced pressure and diluted with 2:1 petroleum ether:ethyl acetate (1 L). The resulting mixture was filtered, the filter cake was washed with 2:1 petroleum ether:ethyl acetate (1 L) and the filtrate was concentrated under reduced pressure. The residue was purified by silica gel column chromatography eluting with petroleum ether:ethyl acetate (3:1) to afford the title compound. LRMS m/z: (M+H)⁺ calculated 232.2; found 232.2.

Step 3: tert-Butyl (R)-4-formyl-2,2-dimethyloxazolidine-3-carboxylate

To a stirred solution of oxalyl chloride (469 g, 3700 mmol, 1.50 equiv) in DCM (3 L) was added a solution of DMSO (578 g, 7390 mmol, 3.00 equiv) in DCM (1 L) at −78° C. under nitrogen atmosphere. To the above, a solution of tert-butyl (4S)-4-(hydroxymethyl)-2,2-dimethyl-1,3-oxazolidine-3-carboxylate (570 g, 2460 mmol, 1.00 equiv) in DCM (5 L) was added dropwise at −60° C. The mixture was stirred for 30 min at −60° C. under nitrogen atmosphere. A solution of TEA (1500 g, 14800 mmol, 6.00 equiv) in DCM (3 L) was added to the above solution at −50° C. and the resulting mixture was stirred for 30 min at −45° C. under nitrogen atmosphere. The resulting mixture was stirred at 0° C. for 1 h. The reaction was quenched by the addition of HCl (6 L, 0.5 M) at room temperature. The resulting mixture was extracted with CH₂Cl₂ (2×1 L). The combined organic layers were washed with water (1×1 L) and dried over anhydrous Na₂SO₄. The mixture was filtered and the filtrate was concentrated under reduced pressure. The residue was purified by silica gel column chromatography, eluted with petroleum ether:ethyl acetate (10:1) to afford the title compound.

Step 4: tert-Butyl (S,E)-2,2-dimethyl-4-(3-oxobut-1-en-1-yl) oxazolidine-3-carboxylate

A mixture of tert-butyl 4-formyl-2,2-dimethyl-1,3-oxazolidine-3-carboxylate (550 g, 2400 mmol, 1.00 equiv.) and 1-(triphenyl-λ⁵-phosphanylidene)propan-2-one (802 g, 2520 mmol, 1.05 equiv.) in THF (7.5 L) was stirred for overnight at 50° C. under nitrogen atmosphere. The resulting mixture was concentrated under reduced pressure and diluted with 4:1 petroleum ether:ethyl acetate (2 L). The mixture was filtered, the filter cake was washed with 4:1 petroleum ether:ethyl acetate (1 L), and the filtrate was concentrated under reduced pressure. The residue was purified by silica gel column chromatography eluting with petroleum ether:ethyl acetate (6:1) to afford the title compound. LRMS m/z: (M+H)⁺ calculated 270.2; found 270.2.

Step 5: tert-Butyl(S)-2,2-dimethyl-4-(3-oxobutyl) oxazolidine-3-carboxylate

To a stirred solution of tert-butyl (4S)-2,2-dimethyl-4-[(1E)-3-oxobut-1-en-1-yl]-1,3-oxazolidine-3-carboxylate (580 g, 2150 mmol, 1.00 equiv) in MeOH (5 L) was added Pd/C (57.3 g, 538 mmol, 0.25 equiv) at room temperature. The mixture was placed under an atmosphere of H₂ and stirred overnight at room temperature. The resulting mixture was filtered, the filter cake was washed with MeOH (200 mL), and the filtrate was concentrated under reduced pressure to afford the title compound. LRMS m/z: (M+H)⁺ calculated 272.2; found 272.2.

Step 6: tert-Butyl(S)-4-(3,3-difluorobutyl)-2,2-dimethyloxazolidine-3-carboxylate

A mixture of tert-butyl (4S)-2,2-dimethyl-4-(3-oxobutyl)-1,3-oxazolidine-3-carboxylate (570 g, 2100 mmol, 1.00 equiv) in diethyl(trifluoro-λ⁴-sulfanyl)amine (4000 mL) was stirred at room temperature under air atmosphere for 48 h. The resulting mixture was diluted with Et₂O (6 L). The reaction was quenched with saturated NaHCO₃ (3 L) at 0° C. The resulting mixture was extracted with EtOAc (2×2 L). The combined organic layers were washed with sat. aq. NaHCO₃ (1 L) and dried over anhydrous Na₂SO₄. After filtration, the filtrate was concentrated under reduced pressure. The residue was purified by silica gel column chromatography eluting with petroleum ether:ethyl acetate (7:1) to afford the title compound. LRMS m/z: (M+H)⁺ calculated 294.2; found 294.2.

Step 7: Benzyl(S)-(5,5-difluoro-1-hydroxyhexan-2-yl)carbamate

To a stirred solution of tert-butyl (4S)-4-(3,3-difluorobutyl)-2,2-dimethyl-1,3-oxazolidine-3-carboxylate (355 g, 1210 mmol, 1.00 equiv.) in MeOH (2 L) was added HCl (441 g, 12100 mmol, 10.00 equiv.) dropwise at room temperature. The reaction mixture was stirred for 30 min at room temperature. The mixture was concentrated under reduced pressure, then taken up in THF (4 L) and H₂O (2 L) at room temperature. K₂CO₃ (335 g, 2420 mmol, 2.00 equiv.) was added. To the above mixture was added benzyl chloroformate (248 g, 1450 mmol, 1.20 equiv.) dropwise at 0° C. The resulting mixture was stirred for additional 2 h at room temperature. The resulting mixture was extracted with EtOAc (3×1 L). The combined organic layers were washed with water (1 L) and dried over anhydrous Na₂SO₄. The mixture was filtered and the filtrate was concentrated under reduced pressure. The residue was purified by silica gel column chromatography eluting with petroleum ether:ethyl acetate (1:1) to afford title compound. LRMS m/z: (M+H)⁺ calculated 288.1; found 288.1.

Step 8: Benzyl(S)-(5,5-difluoro-1-oxohexan-2-yl)carbamate

To a stirred solution of benzyl N-[(2S)-5,5-difluoro-1-hydroxyhexan-2-yl]carbamate (290 g, 1010 mmol, 1.00 equiv) in CH₂Cl₂ (3 L) was added Dess-Martin periodinane (514 g, 1210 mmol, 1.20 equiv) in portions at room temperature under air atmosphere. The resulting mixture was stirred overnight at room temperature. The reaction was quenched with saturated Na₂S203 (aq.) and saturated NaHCO₃ (aq.) at 0° C. The resulting mixture was extracted with CH₂Cl₂ (2×2 L). The combined organic layers were washed with water (1 L) and dried over anhydrous Na₂SO₄. The mixture was filtered and the filtrate was concentrated under reduced pressure. The residue was purified by silica gel column chromatography eluting with petroleum ether:ethyl acetate (3:1) to afford title compound. LRMS m/z: (M+H)⁺ calculated 286.1; found 286.1.

Step 9: benzyl(S)-(5,5-difluoro-1,1-dimethoxyhexan-2-yl)carbamate

To a stirred solution of benzyl N-(5,5-difluoro-1-oxohexan-2-yl)carbamate (176 g, 617 mmol, 1.00 equiv) and trimethyl orthoformate (78.6 g, 740 mmol, 1.20 equiv) in MeOH (1.5 L) was added para-toluene sulfonate (10.6 g, 61.7 mmol, 0.10 equiv) in portions at room temperature. The resulting mixture was stirred overnight at room temperature. The resulting mixture was concentrated under reduced pressure. The residue was dissolved in EtOAc (2 L) and saturated aqueous NaHCO₃ (1 L). The resulting mixture was extracted with EtOAc (2×1.5 L). The combined organic layers were dried over anhydrous Na₂SO₄. The mixture as filtered and the filtrate was concentrated under reduced pressure. The residue was purified by trituration with 3:1 petroleum ether:ethyl acetate (1 L) and the solids were collected by filtration. The crude product was purified by SFC on a Lux Cellulose-2 5 μm 30×250 mm column eluting with 15% (1:1 isopropanol: hexanes)/CO₂ at 100 bar, monitoring at 220 nm to afford the title compound. LRMS m/z: (M+H)⁺ calculated 332.2; found 332.2.

Step 10: (S)-5,5-difluoro-1,1-dimethoxyhexan-2-amine

To a stirred solution of benzyl (S)-(5,5-difluoro-1,1-dimethoxyhexan-2-yl)carbamate (1.5 g, 4.53 mmol) in EtOH (34.8 ml) was added 20 wt % Pearlman's Catalyst (0.318 g, 0.453 mmol). The flask was purged, placed under H_{2(g)} using a balloon and stirred under atmospheric H_{2(g)} at RT for 1 hr. The mixture was filtered carefully through a prepacked celite filter and the catalyst was washed with EtOAc. The filtrate was concentrated to give the title compound. LRMS m/z: (M+H)⁺ calculated 198.1; found 198.2.

### Intermediate 5

### (3S)-3-amino-6,6-difluoro-2-hydroxyheptanamide

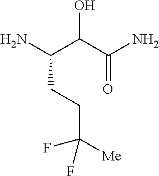 [SMILES: CC(F)(F)CC[C@H](N)C(O)C(N)=O]

Step 1: Benzyl(S)-(5,5-difluoro-1-oxohexan-2-yl)carbamate

A solution of benzyl(S)-(5,5-difluoro-1,1-dimethoxyhexan-2-yl)carbamate (700 mg, 2.11 mmol) in acetone (7.04 mL)/Water (7.04 mL) was treated with Dowex® 50WX8 Hydrogen Form 200-400 mesh (2.1 g) and then shaken and heated to 70° C. for 18 hours. The mixture was filtered and then the solvent was removed under reduced pressure. The residue was dissolved in EtOAc and then washed with saturated aqueous NaHCO₃ and brine, dried (MgSO₄), and filtered. The solvent was removed under reduced pressure to give the title compound. LRMS m/z: (M+H)⁺ calculated 286.1; found 286.2.

Step 2: Benzyl((2S)-1-cyano-5,5-difluoro-1-hydroxyhexan-2-yl)carbamate

To a stirred solution of benzyl (S)-(5,5-difluoro-1-oxohexan-2-yl)carbamate (125 mg, 0.438 mmol) and MeOH (2.19 mL) at 0° C. was added cesium fluoride (66.6 mg, 0.438 mmol) followed by dropwise addition of trimethylsilyl cyanide (147 μL, 1.10 mmol). After 30 minutes the cooling bath was removed and the solution was stirred overnight. The reaction was quenched with saturated aqueous NaHCO₃ and then extracted with EtOAc. The organic portion was washed with brine, dried (MgSO₄), and filtered. The solvent was removed under reduced pressure to give the title compound. LRMS m/z: (M+Na)⁺ calculated 335.1; found 335.1.

Step 3: Benzyl((3S)-1-amino-6,6-difluoro-2-hydroxy-1-oxoheptan-3-yl)carbamate

To a stirred solution of benzyl ((2S)-1-cyano-5,5-difluoro-1-hydroxyhexan-2-yl)carbamate (130 mg, 0.416 mmol) and MeOH (2.08 mL) at ambient temperature was added lithium hydroxide, H₂O (21.8 mg, 0.520 mmol) followed by hydrogen peroxide (425 μL, 4.16 mmol) and the mixture was stirred overnight. The reaction was quenched with saturated aqueous sodium thiosulfate and then extracted with EtOAc. The organic portion was washed with brine, dried (MgSO₄), and filtered, and the solvent was removed under reduced pressure. DMSO (5 mL) was added to the residue, which was filtered and the filtrate purified by reverse-phase HPLC (30×150 mm, Waters SunFire® OBD 10 μm), eluting with a gradient of 5-95% Acetonitrile/Water+0.1% TFA over 15 minutes at 43 mL/min. The fractions that contained product were combined, basified with saturated aqueous NaHCO₃, and then extracted with CH₂Cl₂. The organic portion was dried (MgSO₄) and filtered, and the solvent was removed under reduced pressure to give the title compound. LRMS m/z: (M+H)⁺ calculated 331.1; found 331.3.

Step 4: (3S)-3-amino-6,6-difluoro-2-hydroxyheptanamide

A solution of benzyl ((3S)-1-amino-6,6-difluoro-2-hydroxy-1-oxoheptan-3-yl)carbamate (100 mg, 0.303 mmol) in EtOH (0.250 mL)/EtOAc (1.26 mL) was treated with 20% palladium hydroxide (21.3 mg, 0.030 mmol) and then stirred under 1 atm H₂ for 1 hour. The mixture was filtered through celite and the celite pad was washed with EtOH. The filtrate was concentrated under reduced pressure to give the title compound. LRMS m/z: (M+H)⁺ calculated 197.2; found 197.1.

### Intermediate 6

### (3S)-3-amino-6-fluoro-2-hydroxy-N-methylheptanamide

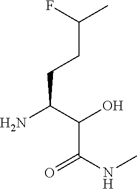

Step 1: methyl (S)-2-((tert-butoxycarbonyl)amino)hex-5-enoate

methyl (S)-2-aminohex-5-enoate (1.7 g, 7.05 mmol), DMAP (0.017 g, 0.141 mmol) and TEA (1.179 mL, 8.46 mmol) were dissolved in DCM (20 mL) and MeOH (20 mL). To this mixture was added BOC-Anhydride (1.964 mL, 8.46 mmol) dropwise at 25° C. within 2 min. The mixture was stirred for 16 h at 25° C. LC/MS showed the starting material was consumed and desired MS was found. The reaction mixture was evaporated. The residue was purified by flash silica gel chromatography (ISCO®, 12 g SepaFlash® Silica Flash Column, eluent of 0-8% ethyl acetate/pet. ether gradient @ 40 mL/min) to give the title compound. LRMS m/z: (M+H-Boc)⁺ calculated 144.1; found 144.2.

Step 2: methyl (2S)-2-((tert-butoxycarbonyl)amino)-5-fluorohexanoate

Iron(III) oxalate hexahydrate (1.989 g, 4.11 mmol) was stirred in Water (82 mL) until completely dissolved (typically 1-2 h). The clear yellow solution was cooled to 0° C. and degassed for 10 min. 1-chloromethyl-4-fluoro-1,4-diazoniabicyclo[2.2.2]octane bis(tetrafluoroborate) (1.456 g, 4.11 mmol) and MeCN (82 mL) were added to the reaction mixture. A solution of methyl (S)-2-((tert-butoxycarbonyl)amino)hex-5-enoate (0.5 g, 2.055 mmol) in MeCN (82 mL) was transferred by pipet to the reaction mixture and NaBH₄ (250 mg) was added to the mixture at 0° C. After 2 min, the reaction mixture was treated with an additional portion of NaBH₄ (250 mg). The resulting mixture was stirred for 30 min before being quenched by addition of 28-30% aqueous NH₄OH (20 mL). The mixture was extracted with 10% MeOH in DCM (3×30 mL) and the organic layer was dried over Na₂SO₄ and concentrated under reduced pressure. The residue was purified by flash silica gel chromatography (ISCO®, 4 g SepaFlash® Silica Flash Column, eluent of 15% ethyl acetate/pet. ether gradient @ 30 mL/min) to give the title compound. LRMS m/z: (M+H−Boc)⁺ calculated 164.1; found 164.1.

¹H NMR (400 MHz, CHLOROFORM-d) δ 5.03 (br d, J=7.2 Hz, 1H), 4.83-4.49 (m, 1H), 4.41-4.20 (m, 1H), 3.75 (s, 3H), 2.04-1.87 (m, 1H), 1.58 (br s, 4H), 1.55-1.46 (m, 1H), 1.44 (s, 9H), 1.35 (d, J=6.1 Hz, 1H).

¹⁹F NMR (376 MHz, CHLOROFORM-d) δ −173.60-−174.30 (m, 1F).

Step 3: tert-butyl ((2S)-5-fluoro-1-oxohexan-2-yl)carbamate

To a solution of methyl (2S)-2-((tert-butoxycarbonyl)amino)-5-fluorohexanoate (0.55 g, 2.089 mmol) in THF (10 mL) at −78° C. was added DIBAL-H (10.44 mL, 10.44 mmol, 1 M in c-hexane) dropwise. The mixture was stirred at −78° C. for 3 h. LC/MS showed the starting material was consumed and desired product was found. MeOH (20 mL) was added to the mixture at −78° C. and the mixture was stirred for 15 min at 25° C. and further quenched with sat. Rochelles salt (10 mL), diluted with EtOAc (10 mL) and removed from the cooling bath and stirred for 0.5 h at 25° C. The mixture was extracted with EtOAc (3×20 mL) and the combined organic phases were washed with brine (2×10 mL), dried over Na₂SO₄, filtered and concentrated to give the title compound which was used for next step without further purification. LRMS m/z: (M+H−C4H8)⁺ calculated 178.1; found 178.1

Step 4: (3S)-3-((tert-butoxycarbonyl)amino)-6-fluoro-1-(methylamino)-1-oxoheptan-2-yl acetate

tert-butyl ((2S)-5-fluoro-1-oxohexan-2-yl)carbamate (500 mg, 2.143 mmol) and isocyanomethane (0.128 mL, 2.358 mmol) in DCM (5 mL) was added to acetic acid (0.147 mL, 2.57 mmol). The mixture was stirred for 16 h at 25° C. LC/MS showed the starting material was consumed and desired product was found. Water (10 mL) was added to the mixture and the mixture was extrated with DCM (3×15 mL). The organic layer was washed with brine (10 mL), dried over MgSO₄, filtered and concentrated to give the title compound which was used in the next step without purification. LRMS m/z: (M+H−Boc)⁺ calculated 235.1; found 235.1.

Step 5: tert-butyl ((3S)-6-fluoro-2-hydroxy-1-(methylamino)-1-oxoheptan-3-yl)carbamate

To a solution of (3S)-3-((tert-butoxycarbonyl)amino)-6-fluoro-1-(methylamino)-1-oxoheptan-2-yl acetate (600 mg, 1.794 mmol) in MeOH (18 mL) and Water (6 mL) was added lithium hydroxide monohydrate (151 mg, 3.59 mmol). The mixture was stirred at 25° C. for 2 h. LC/MS showed the starting material was consumed and desired MS was found. The mixture was concentrated. Water (10 mL) was added to the mixture. The mixture was adjusted to pH=5 by 1N HCl. The mixture was extracted with EtOAc (3×15 mL) and the combined organic phases were washed with brine (2×20 mL), dried over Na₂SO₄, filtered and concentrated in vacuo. The residue was purified directly by RP-HPLC (Column: Welch Xtimate C18 150*25 mm*5 μm, Condition: water (TFA)-ACN Begin B 15 End B 45 Gradient Time (min) 11 100% B Hold Time (min) 2 Flow Rate (mL/min) 25) to give the title compound. LRMS m/z: (M+H−Boc)⁺ calculated 193.1; found 193.1.

¹H NMR (400 MHz, CHLOROFORM-d) δ 7.05-6.61 (m, 1H), 5.49-4.92 (m, 1H), 4.87-4.48 (m, 1H), 4.35-4.05 (m, 1H), 3.92-3.67 (m, 1H), 2.95-2.74 (m, 3H), 1.98-1.49 (m, 5H), 1.48-1.13 (m, 11H).

¹⁹F NMR (376 MHz, CHLOROFORM-d) δ −170.40-−175.84 (m, 1F).

Step 6: (3S)-3-amino-6-fluoro-2-hydroxy-N-methylheptanamide

A solution of tert-butyl ((3S)-6-fluoro-2-hydroxy-1-(methylamino)-1-oxoheptan-3-yl)carbamate (220 mg, 0.753 mmol) in 4M HCl/dioxane (753 μL, 3.01 mmol) was stirred at 25° C. for 1 h. LC/MS showed that the starting material was used up and the mixture was concentrated to give (3S)-3-amino-6-fluoro-2-hydroxy-N-methylheptanamide (130 mg, 0.676 mmol, 90% yield) as a white solid, which was used for next step without further purification. LRMS m/z: (M+H)⁺ calculated 193.1; found 193.1.

### EXAMPLES

### Example 1

### (S)—N-(4-chloro-2-((6,6-difluoro-1-(methylamino)-1,2-dioxoheptan-3-yl)carbamoyl)phenyl)-2-(trifluoromethyl)isonicotinamide

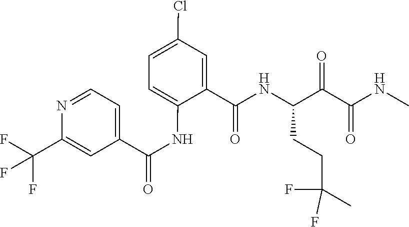 [SMILES: CNC(=O)C(=O)[C@H](CCC(C)(F)F)NC(=O)c1cc(Cl)ccc1NC(=O)c1ccnc(C(F)(F)F)c1]

Step 1: methyl 5-chloro-2-(2-(trifluoromethyl)isonicotinamido)benzoate

To a vial containing methyl 2-amino-5-chlorobenzoate (354 mg, 1.907 mmol) and 2-(trifluoromethyl)isonicotinic acid (507 mg, 2.65 mmol) was added 7-azabenzotriazol-1-yloxytris(dimethylamino)phosphonium hexafluorophosphate (2.13 g, 4.81 mmol) followed by NMP (6 mL) and finally DIPEA (850 μL, 4.87 mmol). The reaction mixture was then capped and heated immediately to 85° C. in the hood. Followed by LC/MS. After 1 night at 85° C., the reaction mixture was diluted with 200 μL MeOH, then purified (without workup) by reverse phase chromatography (25-100% MeCN/H₂O; 0.1% TFA modifier; 30 min gradient; Waters 50×250 mm Sunfire 5 micron C18 column; Flow=118.1 mL/min). The desired fractions were concentrated then dissolved in DCM/MeOH and concentrated to give the title compound. LRMS m/z: (M+H)⁺ calculated 359.7; found 359.1.

Step 2: 5-chloro-2-(2-(trifluoromethyl)isonicotinamido)benzoic acid

To a flask containing methyl 5-chloro-2-(2-(trifluoromethyl)isonicotinamido)benzoate (432 mg, 1.204 mmol) was added MeOH (10 mL) then Water (4 mL) and finally 5N sodium hydroxide (650 μL, 3.25 mmol). The reaction mixture was then capped (not under N₂) and stirred at room temperature. After NaOH was added the mixture was still a suspension so DCM (3 mL) was added which immediately solubilized the mixture. This was followed by LC/MS. After ~1.75 hrs at room temperature the reaction mixture was diluted/acidified with 1N HCl, then suspended in EtOAc, washed with 1N HCl, then brine. Organics were dried over Na₂SO₄, filtered and concentrated to give the title compound which was used without further purification. LRMS m/z: (M+H)⁺ calculated 345.6; found 345.0.

Step 3: N-(4-chloro-2-(((2S,3S)-6,6-difluoro-2-hydroxy-1-(methylamino)-1-oxoheptan-3-yl)carbamoyl)phenyl)-2-(trifluoromethyl)isonicotinamide

To a vial containing 5-chloro-2-(2-(trifluoromethyl)isonicotinamido)benzoic acid (110 mg, 0.319 mmol) and (2S,3S)-3-amino-6,6-difluoro-2-hydroxy-N-methylheptanamide hydrochloride (125 mg, 0.507 mmol) was added 7-azabenzotriazol-1-yloxytris (dimethylamino)phosphonium hexafluorophosphate (217 mg, 0.490 mmol) followed by NMP (1.5 mL) and finally DIPEA (140 μL, 0.802 mmol). The reaction mixture was then capped and heated immediately to 85° C. in the hood. This was followed by LC/MS. After 1 night at 85° C., the reaction mixture was diluted with 200 μL MeOH, and then purified (without workup) by reverse phase chromatography (10-70% MeCN/H2O; 0.1% TFA modifier; 20 min gradient; Waters 30×150 mm Sunfire 5 micron C18 column; Flow=42.5 mL/min). The desired fractions were diluted with MeOH and concentrated to give the title compound. LRMS m/z: (M+H)⁺ calculated 537.1; found 537.1.

Step 4: (S)—N-(4-chloro-2-((6,6-difluoro-1-(methylamino)-1,2-dioxoheptan-3-yl)carbamoyl)phenyl)-2-(trifluoromethyl)isonicotinamide

To a flask containing N-(4-chloro-2-(((2S,3S)-6,6-difluoro-2-hydroxy-1-(methylamino)-1-oxoheptan-3-yl)carbamoyl)phenyl)-2-(trifluoromethyl)isonicotinamide (108 mg, 0.201 mmol) was added Dess-MartinPeriodinane (168 mg, 0.396 mmol) and sodium (33 mg, 0.393 mmol) and finally DCM (5 mL). The reaction mixture was then capped and stirred at room temperature. This was followed by LC/MS. After 45 min the reaction mixture was quenched/diluted with 4 mL of saturated sodium thiosulfate followed by 1 mL water, then diluted with ~10 mL EtOAc and stirred for ~10 minutes (became clear/solubilized), The mixture was then suspended in EtOAc, washed with saturated sodium thiosulfate, then water, and then brine. The organic layer was then dried over Na₂SO₄, filtered and concentrated. Purification was performed by silica gel chromatography (0-80% EtOAc/Hex; 14 CV; 40 g ISCO). The desired fractions were concentrated and then re-purified by silica gel chromatography (0-10% IPA/DCM 14 CV; 40 g ISCO). The desired fractions were concentrated to give the title compound. LRMS m/z: (M+H)⁺ calculated 535.1; found 535.1.

¹H NMR (500 MHz, DMSO-d6) δ 9.29 (d, J=6.9 Hz, 1H), 9.04 (d, J=5.0 Hz, 1H), 8.74-8.64 (m, 1H), 8.32 (d, J=8.9 Hz, 1H), 8.22 (s, 1H), 8.08 (d, J=4.9 Hz, 1H), 7.93 (d, J=2.4 Hz, 1H), 7.72 (dd, J=8.8, 2.4 Hz, 1H), 5.18-5.08 (m, 1H), 2.64 (d, J=4.8 Hz, 3H), 2.13-1.92 (m, 3H), 1.86-1.73 (m, 1H), 1.58 (t, J=19.0 Hz, 3H).

### Example 2

### (S)-5-chloro-N-(6,6-difluoro-1-(methylamino)-1,2-dioxoheptan-3-yl)-2-(3-(trifluoromethyl)bicyclo[1.1.1]pentane-1-carboxamido)nicotinamide

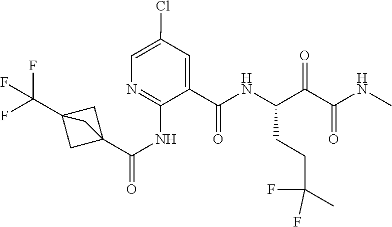 [SMILES: CNC(=O)C(=O)[C@H](CCC(C)(F)F)NC(=O)c1cc(Cl)cnc1NC(=O)C12CC(C(F)(F)F)(C1)C2]

Step 1: methyl 5-chloro-2-(3-(trifluoromethyl)bicyclo[1.1.1]pentane-1-carboxamido)nicotinate

To a vial containing methyl 2-bromo-5-chloronicotinate (630 mg, 2.52 mmol) and 3-(trifluoromethyl)bicyclo[1.1.1]pentane-1-carboxamide (570 mg, 3.18 mmol) was added cesium carbonate (2.65 g, 8.13 mmol), then XantPhos Pd G3 (405 mg, 0.427 mmol). The vial was then capped and anhydrous Dioxane (10 mL) was added under an atmosphere of nitrogen. Then N₂ was bubbled through the reaction mixture for 20 seconds which was then heated to 85° C. in the hood. This was followed by LC/MS. After ~20 minutes the reaction mixture was suspended in EtOAc and diluted with water, then washed with saturated NaHCO₃, and then brine. Organics were dried over Na₂SO₄, filtered and concentrated. Purification was performed by silica gel chromatography (0-80% EtOAc/Hex; 14 CV; 80 g ISCO). The desired fractions were concentrated to give the title compound. LRMS m/z: (M+H)+ calculated 349.0; found 349.1.

Step 2: 5-chloro-2-(3-(trifluoromethyl)bicyclo[1.1.1]pentane-1-carboxamido)nicotinic acid

To a flask containing methyl 5-chloro-2-(3-(trifluoromethyl)bicyclo[1.1.1]pentane-1-carboxamido)nicotinate (634 mg, 1.818 mmol) was added MeOH (6 mL) then water (3 mL) and finally 5N sodium hydroxide (750 μL, 3.75 mmol). The reaction mixture was then capped (not under N₂) and stirred at room temperature. This was followed by LC/MS. After ~30 min at room temperature the reaction mixture was diluted/acidified with 1N HCl, then suspended in EtOAc, washed with 1N HCl, and then brine. Organics were dried over Na₂SO₄, filtered and concentrated to give the title compound which was used without further purification. LRMS m/z: (M+H)+ calculated 335.0; found 335.0.

Step 3:5-chloro-N-((2S,3S)-6,6-difluoro-2-hydroxy-1-(methylamino)-1-oxoheptan-3-yl)-2-(3-(trifluoromethyl)bicyclo[1.1.1]pentane-1-carboxamido)nicotinamide

To a vial containing 5-chloro-2-(3-(trifluoromethyl)bicyclo[1.1.1]pentane-1-carboxamido)nicotinic acid (171 mg, 0.511 mmol) and (2S,3S)-3-amino-6,6-difluoro-2-hydroxy-N-methylheptanamide hydrochloride (186 mg, 0.754 mmol) was added 7-azabenzotriazol-1-yloxytris(dimethylamino)phosphonium hexafluorophosphate (362 mg, 0.817 mmol) followed by DMF (2.5 mL) and finally DIPEA (240 μL, 1.374 mmol). The reaction mixture was then capped and heated immediately to 85° C. in the hood. This was followed by LC/MS. After 1 night at 85° C., the reaction mixture was transferred to a test tube with MeOH/DCM, and then partially concentrated. The product was then purified (without workup) by reverse phase chromatography (10-65% MeCN/H2O; 0.1% TFA modifier; 30 min gradient; Waters 50×250 mm Sunfire 5 micron C18 column; Flow=118.1 mL/min). The desired fractions were suspended in EtOAc, washed with saturated NaHCO₃, then water, and then brine. The organic layer was then dried over Na₂SO₄, filtered and concentrated to give the title compound. LRMS m/z: (M+H)+ calculated 527.1; found 527.2.

Step 4: (S)-5-chloro-N-(6,6-difluoro-1-(methylamino)-1,2-dioxoheptan-3-yl)-2-(3-(trifluoromethyl)bicyclo[1.1.1]pentane-1-carboxamido)nicotinamide

To a vial containing 5-chloro-N-((2S,3S)-6,6-difluoro-2-hydroxy-1-(methylamino)-1-oxoheptan-3-yl)-2-(3-(trifluoromethyl)bicyclo[1.1.1]pentane-1-carboxamido)nicotinamide (120 mg, 0.228 mmol) was added Dess-MartinPeriodinane (173 mg, 0.408 mmol) and sodium bicarbonate (43 mg, 0.512 mmol) and finally DCM (4 mL). The reaction mixture was then capped and stirred at room temperature. This was followed by LC/MS. After 45 min the reaction mixture was quenched/diluted with 4 mL of saturated sodium thiosulfate followed by 1 mL water, and then diluted with ~10 mL EtOAc. The mixture was stirred for 10 minutes then suspended in EtOAc, and washed with saturated sodium thiosulfate, then water, and then brine. The organic layer was then dried over Na₂SO₄, filtered and concentrated. Purification was performed by silica gel chromatography (0-100% EtOAc/Hex; 14 CV; 40 g ISCO). The desired fractions were concentrated and then re-purified by silica gel chromatography (10-100% EtOAc/Hex; 14 CV; 40 g ISCO). The desired fractions were concentrated to give the title compound. LRMS m/z: (M+H)+ calculated 525.1; found 525.1.

¹H NMR (500 MHz, DMSO-d6) δ 10.83 (s, 1H), 9.06 (d, J=7.2 Hz, 1H), 8.70-8.64 (m, 1H), 8.59 (d, J=2.5 Hz, 1H), 8.05 (d, J=2.5 Hz, 1H), 5.09-5.02 (m, 1H), 2.67 (d, J=4.8 Hz, 3H), 2.26 (s, 6H), 2.19-2.06 (m, 2H), 2.05-1.93 (m, 2H), 1.79-1.70 (m, 1H), 1.63 (t, J=19.0 Hz, 3H)

### Example 3

### Methyl ((S)-1-((4-chloro-2-(((S)-1-(cyclopropylamino)-6,6-difluoro-1,2-dioxoheptan-3-yl)carbamoyl)phenyl)amino)-3,3-dimethyl-1-oxobutan-2-yl)carbamate

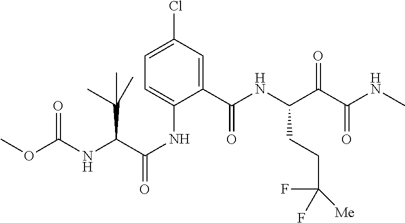

Step 1: Methyl(S)-5-chloro-2-(2-((methoxycarbonyl)amino)-3,3-dimethylbutanamido)benzoate

To a stirred solution of(S)-2-((methoxycarbonyl)amino)-3,3-dimethylbutanoic acid (500 mg, 2.64 mmol), methyl 2-amino-5-chlorobenzoate (490 mg, 2.64 mmol) and pyridine (2643 μl) at ambient temperature was added EDC (760 mg, 3.96 mmol). The mixture was stirred for 18 hours. (S)-2-((methoxycarbonyl)amino)-3,3-dimethylbutanoic acid (500 mg, 2.64 mmol) and EDC (760 mg, 3.96 mmol) were added and the mixture was heated to 60° C. for 1 hour. The mixture was allowed to cool to ambient temperature and then was quenched with 1N HCl and then extracted with EtOAc. The combined organic portions were washed with sat aq NaHCO₃, brine, dried (MgSO₄), filtered and the solvent was removed under reduced pressure. The residue was purified by column chromatography on silica gel (ISCO 40 gram), eluting with hexanes to EtOAc over 12 column volumes to give the title compound. LRMS m/z: (M+H)⁺ calculated 357.1; found 357.2.

Step 2: (S)-5-Chloro-2-(2-((methoxycarbonyl)amino)-3,3-dimethylbutanamido)benzoic acid

To a stirred solution of methyl (S)-5-chloro-2-(2-((methoxycarbonyl)amino)-3,3-dimethylbutanamido)benzoate (800 mg, 2.242 mmol) and methanol (11.200 ml) was added 1N NaOH (6.73 ml, 6.73 mmol). The mixture was stirred for 1 hour at ambient temperature. The mixture was acidified with 1N HCl and then extracted with EtOAc. The organic portion was separated, washed with brine, dried (MgSO₄) and then the solvent was removed under reduced pressure to give the title compound which was used without further purification. LRMS m/z: (M+H)⁺ calculated 343.8; found 343.3.

Step 3: Methyl ((2S)-1-((4-chloro-2-(((3S)-1-(cyclopropylamino)-6,6-difluoro-2-hydroxy-1-oxoheptan-3-yl)carbamoyl)phenyl)amino)-3,3-dimethyl-1-oxobutan-2-yl)carbamate

To a stirred solution of(S)-5-chloro-2-(2-((methoxycarbonyl)amino)-3,3-dimethylbutanamido)benzoic acid (100 mg, 0.292 mmol), (3S)-3-amino-N-cyclopropyl-6,6-difluoro-2-hydroxyheptanamide (76 mg, 0.321 mmol) N-methylmorpholine (128 μl, 1.167 mmol) and DMF (1459 μl) was added HATU (139 mg, 0.365 mmol) and the reaction was stirred at 60° C. for 2 hours. The reaction was diluted with DCM and then washed with sat aq NaHCO₃, brine, dried (MgSO₄) and the solvent was removed under reduced pressure. DMSO (3 mL) was added to the residue, which was filtered and the filtrate purified by reverse phase (C-18) HPLC (50×250 mm, Waters SunFire® OBD™ 10 micron) eluting with a gradient of 5-95% Acetonitrile/Water+0.1% TFA over 20 minutes at 118.1 ml/min. The fractions that contained product were combined, basified with sat NaHCO₃ and then extracted with CH₂Cl₂. The organic portion was dried (MgSO₄) and the solvent was removed under reduced pressure to provide the title compound. LRMS m/z: (M+H)⁺ calculated 561.2; found 561.4.

Step 4: Methyl ((S)-1-((4-chloro-2-(((S)-1-(cyclopropylamino)-6,6-difluoro-1,2-dioxoheptan-3-yl)carbamoyl)phenyl)amino)-3,3-dimethyl-1-oxobutan-2-yl)carbamate

Methyl ((2S)-1-((4-chloro-2-(((3S)-1-(cyclopropylamino)-6,6-difluoro-2-hydroxy-1-oxoheptan-3-yl)carbamoyl)phenyl)amino)-3,3-dimethyl-1-oxobutan-2-yl)carbamate (150 mg, 0.267 mmol) was dissolved in DCM (2674 μl), cooled to 0° C. and then treated with sodium bicarbonate (90 mg, 1.069 mmol) followed by Dess-Martin Periodinane (170 mg, 0.401 mmol). The mixture was stirred for 5 minutes at 0° C. and then warmed to RT and the reaction was stirred for another 30 minutes. The mixture was quenched with sat. sodium thiosulfate/sat. NaHCO₃/H₂O and stirred for 15 minutes and then the mixture was extracted with DCM. The aqueous phase was back extracted with DCM. The combined organic phases were dried over Na₂SO₄, filtered and the solvent was removed under reduced pressure. The residue was purified by column chromatography on silica gel (ISCO 24 gram) eluting with hexanes to EtOAc over 12 column volumes to give the title compound. LRMS m/z: (M+H)⁺ calculated 559.2; found 559.4.

¹H NMR (500 MHz, DMSO-d6) δ 11.08 (s, 1H), 9.16 (d, J=7.1 Hz, 1H), 8.79 (d, J=5.1 Hz, 1H), 8.36 (d, J=9.0 Hz, 1H), 7.86 (m, 1H), 7.70-7.48 (m, 2H), 5.22-5.09 (m, 1H), 3.73 (m, 1H), 3.56 (s, 3H), 2.78 (tq, J=9.1, 3.9 Hz, 1H), 2.13-1.91 (m, 3H), 1.80 (dq, J=17.1, 9.8, 7.0 Hz, 1H), 1.63 (t, J=19.0 Hz, 3H), 0.98 (s, 9H), 0.73-0.56 (m, 4H).

### Example 4

### (S)-5-chloro-N-(6,6-difluoro-1-(methylamino)-1,2-dioxoheptan-3-yl)-4-fluoro-2-(4,4,4-trifluorobutanamido)benzamide

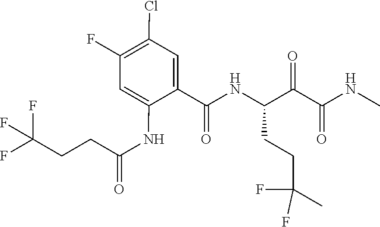 [SMILES: CNC(=O)C(=O)[C@H](CCC(C)(F)F)NC(=O)c1cc(Cl)c(F)cc1NC(=O)CCC(F)(F)F]

Step 1: methyl 5-chloro-4-fluoro-2-(4,4,4-trifluorobutanamido)benzoate

To a vial containing methyl 2-amino-5-chloro-4-fluorobenzoate (571 mg, 2.80 mmol) and 4,4,4-trifluorobutyric acid (519 mg, 3.65 mmol) was added Pyridine (8 mL) and finally POCl₃ (0.400 mL, 4.29 mmol). The reaction mixture was then capped and stirred at room temperature. Followed by LC/MS. After 20 min at room temperature, the reaction mixture was suspended in EtOAc, washed with saturated NaHCO₃, then water, then brine. The organic layer was dried over Na₂SO₄, filtered and concentrated. Purification was performed by silica gel chromatography (0-40% EtOAc/Hex; 14 CV, 80 g ISCO). The desired fractions were concentrated to give the title compound. LRMS m/z: (M+H)+ calculated 328.0; found 328.0.

Step 2: 5-chloro-4-fluoro-2-(4,4,4-trifluorobutanamido)benzoic acid

To a flask containing methyl 5-chloro-4-fluoro-2-(4,4,4-trifluorobutanamido)benzoate (620 mg, 1.892 mmol) was added MeOH (10 mL) then water (4 mL) and finally 5N sodium hydroxide (960 μL, 4.80 mmol). The reaction mixture was then capped and stirred at room temperature. After NaOH was added it was still not well solubilized so DCM (3 mL) was added which immediately solubilized the mixture. This was followed by LC/MS. After 1.5 hrs at room temperature the reaction mixture was diluted with 1N HCl, then suspended in EtOAc and then washed with 1N HCl, then brine. The organic layer was dried over Na₂SO₄, filtered and concentrated to give the title compound which was used without further purification. LRMS m/z: (M+H)+ calculated 314.1.

Step 3: 5-chloro-N-((2S,3S)-6,6-difluoro-2-hydroxy-1-(methylamino)-1-oxoheptan-3-yl)-4-fluoro-2-(4,4,4-trifluorobutanamido)benzamide

To a vial containing 5-chloro-4-fluoro-2-(4,4,4-trifluorobutanamido)benzoic acid (118 mg, 0.376 mmol) and (2S,3S)-3-amino-6,6-difluoro-2-hydroxy-N-methylheptanamide hydrochloride (135 mg, 0.547 mmol) was added 7-azabenzotriazol-1-yloxytris (dimethylamino)phosphonium hexafluorophosphate (290 mg, 0.654 mmol) followed by NMP (1.5 mL) and finally DIPEA (170 μL, 0.973 mmol). The reaction mixture was then capped and heated immediately to 85° C. in the hood. This was followed by LC/MS. After 1 night at 85° C., the reaction mixture was diluted with 200 uL MeOH, and then purified (without workup) by reverse phase chromatography (10-80% MeCN/H2O; 0.1% TFA modifier; 20 min gradient; Waters 30×150 mm Sunfire 5 micron C18 column; Flow=42.5 mL/min). The desired fractions were concentrated to give the title compound. LRMS m/z: (M+H)+ calculated 506.1; found 506.1.

Step 4: (S)-5-chloro-N-(6,6-difluoro-1-(methylamino)-1,2-dioxoheptan-3-yl)-4-fluoro-2-(4,4,4-trifluorobutanamido)benzamide

To a flask containing 5-chloro-N-((2S,3S)-6,6-difluoro-2-hydroxy-1-(methylamino)-1-oxoheptan-3-yl)-4-fluoro-2-(4,4,4-trifluorobutanamido)benzamide (65.5 mg, 0.129 mmol) was added Dess-MartinPeriodinane (106 mg, 0.250 mmol) and sodium bicarbonate (24 mg, 0.286 mmol) followed by DCM (5 mL). The reaction mixture was then capped and stirred at room temperature. This was followed by LC/MS. After 2 hrs the reaction mixture was quenched/diluted with 4 mL of saturated sodium thiosulfate followed by 1 mL water, then diluted with ~10 mL EtOAc and stirred for 10 minutes. The mixture was then suspended in EtOAc, washed with saturated sodium thiosulfate, then water, and then brine. The organic layer was dried over Na₂SO₄, filtered and concentrated. Purification was performed by silica gel chromatography (0-100% EtOAc/Hex; 14 CV; 40 g ISCO). The desired fractions were concentrated. The resulting material was re-purified by silica gel chromatography (0-10% IPA/DCM; 40 g ISCO). The desired fractions were concentrated to give the title compound. LRMS m/z: (M+H)+ calculated 504.1; found 504.2.

¹H NMR (500 MHz, DMSO-d6) δ 11.02 (s, 1H), 9.21 (d, J=6.8 Hz, 1H), 8.78-8.68 (m, 1H) 8.34 (d, J=12.1 Hz, 1H), 8.04 (d, J=8.1 Hz, 1H), 5.15-5.08 (m, 1H), 2.70-2.64 (m, 5H), 2.63-2.54 (m, 2H), 2.16-1.93 (m, 3H), 1.85-1.75 (m, 1H), 1.63 (t, J=19.0 Hz, 3H).

### Example 5

### (S)-5-chloro-N-(1-(cyclopropylamino)-6,6-difluoro-1,2-dioxoheptan-3-yl)-2-(3-(trifluoromethyl)benzamido)nicotinamide

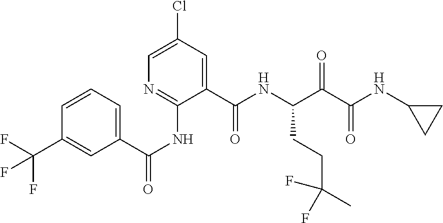 [SMILES: CC(F)(F)CC[C@H](NC(=O)c1cc(Cl)cnc1NC(=O)c1cccc(C(F)(F)F)c1)C(=O)C(=O)NC1CC1]

Step 1: methyl 5-chloro-2-(3-(trifluoromethyl)benzamido)nicotinate

To a vial containing methyl 2-bromo-5-chloronicotinate (768 mg, 3.07 mmol) and 3-(trifluoromethyl)benzamide (716 mg, 3.79 mmol) was added cesium carbonate (3.1 g, 9.51 mmol), then XantPhos Pd G3 (517 mg, 0.545 mmol) and finally anhydrous Dioxane (10 mL) The reaction mixture was then capped (under an atomspher of nitrogen) and heated to 85° C. in the hood. This was followed by LC/MS. After 30 minutes the reaction mixture was suspended in EtOAc and diluted with water, then washed with saturated NaHCO₃, and then brine. The organic layer was dried over Na₂SO₄, filtered and concentrated. Purification was performed by silica gel chromatography (0-40% EtOAc/Hex; 14 CV; 80 g ISCO). The desired fractions were concentrated to give the title compound. LRMS m/z: (M+H)+ calculated 359.0; found 359.1.

Step 2: 5-chloro-2-(3-(trifluoromethyl)benzamido)nicotinic acid

To a flask containing methyl 5-chloro-2-(3-(trifluoromethyl)benzamido)nicotinate (562 mg, 1.567 mmol) was added MeOH (6 mL) then water (3 mL) and finally 5N sodium hydroxide (0.7 mL, 3.50 mmol). The reaction mixture was then capped and stirred at room temperature. This was followed by LC/MS. After 45 min at room temperature the reaction mixture was diluted with EtOAc, then acidified with 1N HCl, separated, and washed with 1N HCl, followed by brine. The organic layer was dried over Na₂SO₄, filtered and concentrated to give the title compound which was used without further purification. LRMS m/z: (M+H)+ calculated 345.0; found 345.1.

Step 3: 5-chloro-N-((2S,3S)-1-(cyclopropylamino)-6,6-difluoro-2-hydroxy-1-oxoheptan-3-yl)-2-(3-(trifluoromethyl)benzamido)nicotinamide

To a vial containing 5-chloro-2-(3-(trifluoromethyl)benzamido)nicotinic acid (121 mg, 0.351 mmol) and (2S,3S)-3-amino-N-cyclopropyl-6,6-difluoro-2-hydroxyheptanamide hydrochloride (138 mg, 0.506 mmol) was added 7-azabenzotriazol-1-yloxytris(dimethylamino)phosphonium hexafluorophosphate (283 mg, 0.638 mmol) followed by DMF (1.5 ml) and finally DIPEA (160 μL, 0.916 mmol). The reaction mixture was then capped and heated to 85° C. in the hood. This was followed by LC/MS. After 1 night at 85° C. the reaction mixture was purified (without workup) by reverse phase chromatography (10-70% MeCN/H2O; 0.1% TFA modifer; 20 min gradient; Waters 30×150 mm Sunfire 5 micron C18 column; Flow=42.5 mL/min). The desired fractions were suspended in EtOAc, washed with saturated NaHCO₃, then water, and then brine. The organic layer was dried over Na₂SO₄, filtered and concentrated to give the title compound. LRMS m/z: (M+H)+ calculated 563.1; found 563.4.

Step 4: (S)-5-chloro-N-(1-(cyclopropylamino)-6,6-difluoro-1,2-dioxoheptan-3-yl)-2-(3-(trifluoromethyl)benzamido)nicotinamide

To a vial containing 5-chloro-N-((2S,3S)-1-(cyclopropylamino)-6,6-difluoro-2-hydroxy-1-oxoheptan-3-yl)-2-(3-(trifluoromethyl)benzamido)nicotinamide (75 mg, 0.133 mmol) was added Dess-MartinPeriodinane (119 mg, 0.281 mmol), sodium bicarbonate (25 mg, 0.298 mmol) and finally DCM (2.5 mL). The reaction mixture was then capped and stirred at room temperature. Followed by LC/MS. After 2 hrs the reaction mixture was quenched/diluted with 3 mL of saturated sodium thiosulfate followed by 1 mL water, then diluted with 5 mL EtOAc, and then stirred for 10 minutes. The reaction mixture was then suspended in EtOAc, washed with saturated sodium thiosulfate, then water, and then brine. The organic layer was dried over Na₂SO₄, filtered and concentrated. Purification was performed by silica gel chromatography (0-100% EtOAc/Hex; 14 CV; 40 g ISCO). The desired fractions were concentrated to give the title compound. LRMS m/z: (M+H)+ calculated 561.1; found 561.3.

¹H NMR (500 MHz, DMSO-d6) δ 11.42 (s, 1H), 9.12 (d, J=7.3 Hz, 1H), 8.74 (d, J=4.8 Hz, 1H), 8.66 (d, J=2.4 Hz, 1H), 8.30 (s, 1H), 8.25 (d, J=7.9 Hz, 1H), 8.05-7.95 (m, 2H), 7.78 (t, J=7.8 Hz, 1H), 4.99-4.89 (m, 1H), 2.75-2.68 (m, 1H), 2.12-1.98 (m, 1H), 1.97-1.80 (m, 2H), 1.75-1.62 (m, 1H), 1.49 (t, J=18.8 Hz, 3H), 0.69-0.60 (m, 2H) 0.57-0.49 (m, 2H).

### Example 6

### methyl (4-chloro-2-(((3S)-6-fluoro-1-(methylamino)-1,2-dioxoheptan-3-yl)carbamoyl)phenyl)carbamate

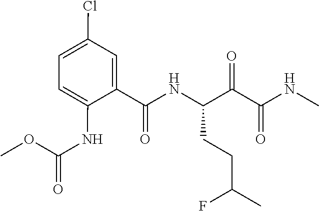

Step 1: 5-chloro-2-((methoxycarbonyl)amino)benzoic acid

To a mixture of 2-amino-5-chlorobenzoic acid (200 mg, 1.166 mmol) and 2,5-dioxopyrrolidin-1-yl methyl carbonate (242 mg, 1.399 mmol) in DCM (5 mL) was added TEA (0.487 mL, 3.50 mmol) at 25° C. to give brown mixture. The resulting mixture was stirred at 50° C. for 3 h. LCMS showed desired MS was found and starting material. The reaction mixture was quenched with water (10 mL) and extracted with DCM (10 mL three times). The combined organic phases were washed with brine (10 mL), dried over anhydrous Na₂SO₄, filtered and concentrated under reduced pressure. The residue was purified by RP-HPLC (Column: Welch Xtimate C18 150*25 mm*5 μm, Condition: water (0.1% TFA)-ACN Begin B 35 End B 65 Gradient Time (min) 11 100% B Hold Time 2 Flow Rate (mL/min) 25) to give the title compound. LRMS m/z: (M+H)+ calculated 230.0; found 230.3.

¹H NMR (400 MHz, MeOD) δ 7.76-7.71 (m, 2H), 7.52 (dd, J=2.1, 8.4 Hz, 1H), 5.78 (s, 1H).

Step 2: methyl (4-chloro-2-(((3S)-6-fluoro-2-hydroxy-1-(methylamino)-1-oxoheptan-3-yl)carbamoyl)phenyl)carbamate

To a solution of 5-chloro-2-((methoxycarbonyl)amino)benzoic acid (71.7 mg, 0.312 mmol), (3S)-3-amino-6-fluoro-2-hydroxy-N-methylheptanamide (60 mg, 0.312 mmol) and N-ethyl-N-isopropylpropan-2-amine (0.164 mL, 0.936 mmol) in DMF (3 mL) was added AOP (166 mg, 0.375 mmol) at 25° C. The reaction mixture was stirred at 25° C. for 16 h. Followed by LC/MS. The mixture was then purified directly (without workup) by RP-HPLC (Column: Boston Prime C18 150 40 mm*5 um, Condition: water (TFA)-ACN Begin B 40 End B 60 Gradient Time (min) 10 100% B Hold Time (min) 25 Flow Rate (mL/min) 25) to give the title compound. LRMS m/z: (M+H)+ calculated 404.1; found 404.1.

SFC Separation by Chiral-SFC (DAICEL CHIRALPAK AD (250 mm*30 mm, 10 um), Condition: 0.1% NH₃—H₂O EtOH Begin B 25 End B 25 Gradient Time (min) 10, 100% B Hold Time (min) 10, Flow Rate (mL/min) 70) to give P1 (Rt=3.31 min) as the first eluting peak, P2 (Rt=3.654 min) as the second eluting peak, P3 (Rt=3.832 min) as the third eluted peak, and P4 (Rt=4.086 min) as the forth eluted peak.

P1, P2, P3, and P4: LRMS m/z: (M+H)+ calculated 404.1; found 404.1.

Step 3: methyl (4-chloro-2-(((3S)-6-fluoro-1-(methylamino)-1,2-dioxoheptan-3-yl)carbamoyl)phenyl)carbamate

To a solution of methyl (4-chloro-2-(((3S)-6-fluoro-2-hydroxy-1-(methylamino)-1-oxoheptan-3-yl)carbamoyl)phenyl)carbamate (15 mg, 0.037 mmol, P3) in DCM (2 mL) was added DMP (47.3 mg, 0.111 mmol). The reaction mixture was stirred for 2 h at 25° C. Followed by LC/MS. The the reaction mixture was quenched with saturated sodium thiosulfate (1 mL)/sat. NaHCO3 (1 mL) and stirred vigorously for 30 min. Then the mixture was extracted with DCM (2 mL). The aqueous phase was re-extracted with DCM (2×2 mL). The combined organic phases were dried over Na₂SO₄, filtered and the filtrate was concentrated under reduced pressure. The resulting residue was purified by RP-HPLC (Column: Welch Xtimate C18 150*25 mm*5 um, Condition: water (0.1% TFA)-ACN Begin B 43 End B 63 Gradient Time (min) 10 100% B Hold Time 2 Flow Rate (mL/min) 25) to give the title compound. LRMS m/z: (M+H)+ calculated 402.1; found 402.0.

¹H NMR (400 MHz, METHANOL-d4) δ 8.27-8.07 (m, 1H), 7.81-7.28 (m, 2H), 4.68-4.55 (m, 1H), 3.86-3.64 (m, 3H), 2.97-2.69 (m, 3H), 2.24-1.94 (m, 1H), 1.92-1.49 (m, 3H), 1.49-1.16 (m, 4H).

### Example 7

### (S)—N-(2-((1-amino-6,6-difluoro-1_2-dioxoheptan-3-yl)carbamoyl)-4-chlorophenyl)-2-(trifluoromethyl)isonicotinamide

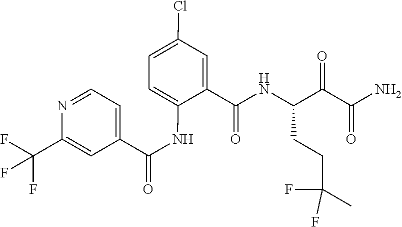 [SMILES: CC(F)(F)CC[C@H](NC(=O)c1cc(Cl)ccc1NC(=O)c1ccnc(C(F)(F)F)c1)C(=O)C(N)=O]

Step 1: methyl 5-chloro-2-(2-(trifluoromethyl)isonicotinamido)benzoate

To a vial containing methyl 2-amino-5-chlorobenzoate (354 mg, 1.907 mmol) and 2-(trifluoromethyl)isonicotinic acid (507 mg, 2.65 mmol) was added 7-azabenzotriazol-1-yloxytris(dimethylamino)phosphonium hexafluorophosphate (2.13 g, 4.81 mmol) followed by NMP (6 mL) and finally DIPEA (850 μL, 4.87 mmol). The reaction mixture was then capped and heated immediately to 85° C. in the hood. This was followed by LC/MS. After 1 night at 85° C., the reaction mixture was diluted with 200 μL MeOH and then purified (without workup) by reverse phase chromatography (25-100% MeCN/H2O; 0.1% TFA modifier; 30 min gradient; Waters 50×250 mm Sunfire 5 micron C18 column; Flow=118.1 mL/min). The desired fractions were concentrated and then dissolved in DCM/MeOH and concentrated to give the title compound. LRMS m/z: (M+H)⁺ calculated 359.7; found 359.1.

Step 2:5-chloro-2-(2-(trifluoromethyl)isonicotinamido)benzoic acid

To a flask containing methyl 5-chloro-2-(2-(trifluoromethyl)isonicotinamido)benzoate (432 mg, 1.204 mmol) was added MeOH (10 mL) then Water (4 mL) and finally 5N sodium hydroxide (650 μL, 3.25 mmol). The reaction mixture was then capped (not under N₂) and stirred at room temperature. After NaOH was added the mixture was still a suspension so DCM (3 mL) was added which immediately solubilized the mixture. This was followed by LC/MS. After ~1.75 hrs at room temperature the reaction mixture was diluted/acidified with 1N HCl, then suspended in EtOAc, washed with 1N HCl, and then brine. Organics were dried over Na₂SO₄, filtered and concentrated to give the title compound which was used without further purification. LRMS m/z: (M+H)⁺ calculated 345.6; found 345.0.

Step 3: preparation of N-(2-(((3S)-1-amino-6,6-difluoro-2-hydroxy-1-oxoheptan-3-yl)carbamoyl)-4-chlorophenyl)-2-(trifluoromethyl)isonicotinamide

To a mixture of 5-chloro-2-(2-(trifluoromethyl)isonicotinamido)benzoic acid (70 mg, 0.203 mmol) and (3S)-3-amino-6,6-difluoro-2-hydroxyheptanamide (50 mg, 0.255 mmol) in DMF (0.5 mL) was added AOP (110 mg, 0.248 mmol), and DIEA (0.1 mL, 0.573 mmol) at 25° C. The resulting mixture was stirred at 25° C. for 2 h. This was followed by LC/MS. Then the reaction mixture was directly purified (without workup) by RP-HPLC (Column: Boston Prime C18 150*40 mm*5 um, Condition: water (0.1% TFA)-ACN Begin B 43 End B 63 Gradient Time (min) 10 100% B Hold Time 25 Flow Rate (mL/min) 25) to give the title compound. LRMS m/z: (M+H)⁺ calculated 523.1; found 523.1.

Step 4: preparation of (S)—N-(2-((1-amino-6,6-difluoro-1,2-dioxoheptan-3-yl)carbamoyl)-4-chlorophenyl)-2-(trifluoromethyl)isonicotinamide

To a mixture of N-(2-(((3S)-1-amino-6,6-difluoro-2-hydroxy-1-oxoheptan-3-yl)carbamoyl)-4-chlorophenyl)-2-(trifluoromethyl)isonicotinamide (70 mg, 0.134 mmol) and NaHCO₃ (45.0 mg, 0.536 mmol) in DCM (1 mL) was added DMP (100 mg, 0.236 mmol), at 25° C. The resulting reaction mixture was stirred at 25° C. for 1 h. This was followed by LC/MS. Then the mixture was filtered and the filtrate was concentrated under reduced pressure. The resulting residue was purified by flash silica gel chromatography (ISCO®, 4 g SepaFlash® Silica Flash Column, eluent of 25% ethyl acetate/pet. ether gradient @50 mL/min) to give the title compound. LRMS m/z: (M+H)⁺ calculated 521.0; found 521.1.

¹H NMR (400 MHz, METHANOL-d4) δ 8.96 (d, J=5.0 Hz, 1H), 8.57 (d, J=8.9 Hz, 1H), 8.28 (s, 1H), 8.10 (d, J=3.6 Hz, 1H), 7.95 (d, J=2.5 Hz, 1H), 7.63 (dd, J=2.5, 8.9 Hz, 1H), 5.33 (dd, J=4.4, 9.4 Hz, 1H), 2.31-2.17 (m, 1H), 2.16-2.02 (m, 1H), 1.99-1.87 (m, 1H), 1.62 (t, J=18.5 Hz, 3H), 1.34 (br d, J=12.2 Hz, 1H).

### Example 57

### (S)-5-chloro-N-(6,6-difluoro-1-(methylamino)-1,2-dioxoheptan-3-yl)-2-(3-((4,4-difluorocyclohexyl)methyl)-3-methylureido)benzamide

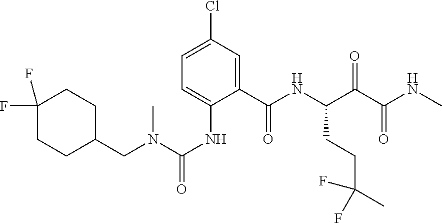 [SMILES: CNC(=O)C(=O)[C@H](CCC(C)(F)F)NC(=O)c1cc(Cl)ccc1NC(=O)N(C)CC1CCC(F)(F)CC1]

Step 1: methyl 5-chloro-2-(3-((4,4-difluorocyclohexyl)methyl)-3-methylureido)benzoate

To a vial containing METHYL 2-AMINO-5-CHLOROBENZOATE (202 mg, 1.088 mmol) was added triphosgene (123 mg, 0.415 mmol), followed by MeCN (4 mL). The reaction mixture was then capped and stirred at room temperature. Followed by LC/MS. After 1 night at room temperature, added DIPEA (775 μL, 4.44 mmol), followed by [(4,4-DIFLUOROCYCLOHEXYL)METHYL](METHYL)AMINE HYDROCHLORIDE (259 mg, 1.297 mmol). Then continued to stir at room temperature. After 1.5 hrs at room temperature the reaction mixture was diluted/quenched with water, then suspended in EtOAc, washed with saturated sodium bicarbonate, then water, then brine. The organic layer was then dried over anhydrous sodium sulfate, then filtered & concentrated. The resulting residue was purified by silica gel chromatography (0-10% IPA/DCM; 80 g ISCO). The desired fractions were concentrated to yield the title compound. LRMS m/z: (M+H)+ calculated 375.1; found 375.2.

Step 2: 5-chloro-2-(3-((4,4-difluorocyclohexyl)methyl)-3-methylureido)benzoic acid

To a flask containing methyl 5-chloro-2-(3-((4,4-difluorocyclohexyl)methyl)-3-methylureido)benzoate (308 mg, 0.822 mmol) was added MeOH (8 mL) followed by DCM (2 mL), then Water (2 mL) & finally 5N sodium hydroxide (425 μL, 2.125 mmol). The reaction mixture was then capped & stirred at room temperature. Followed by LC/MS. After 1.5 hrs at room temperature the reaction mixture was diltued/quenched with 1N HCl (10 mL, 10.00 mmol), then suspended in EtOAc, washed with 1N HCl, followed by brine. The organic layer was then dried over anhydrous sodium sulfate, filtered & concentrted to yield the title compound which was used without further purification. LRMS m/z: (M+H)⁺ calculated 361.7; found 361.2.

Step 3: 5-chloro-N-((2S,3S)-6,6-difluoro-2-hydroxy-1-(methylamino)-1-oxoheptan-3-yl)-2-(3-((4,4-difluorocyclohexyl)methyl)-3-methylureido)benzamide

To a vial containing 5-chloro-2-(3-((4,4-difluorocyclohexyl)methyl)-3-methylureido)benzoic acid (82 mg, 0.227 mmol) & (2S,3S)-3-amino-6,6-difluoro-2-hydroxy-N-methylheptanamide hydrochloride (78 mg, 0.316 mmol) was added 7-AZABENZOTRIAZOL-1-YLOXYTRIS(DIMETHYLAMINO)PHOSPHONIUM HEXAFLUOROPHOSPHATE (156 mg, 0.352 mmol) followed by NMP (1 mL) & finally DIPEA (105 μL, 0.601 mmol). The reaction mixture was then capped and heated to 85° C. in the hood. Followed by LC/MS. After 1 night at 85° C. the reaction mixture was diluted with 300 uL MeOH then purified (without workup) by reverse phase chromatography (10-80% MeCN/H2O; 0.1% TFA modifier; 20 min gradient; Waters 30×150 mm Sunfire 5 micron C18 column; Flow=42.5 mL/min). The desired fractions were concentrated then dissolved in DCM/MeOH & concentrated to yield the title compound. LRMS m/z: (M+H)⁺ calculated 553.9; found 553.4.

Step 4: (S)-5-chloro-N-(6,6-difluoro-1-(methylamino)-1,2-dioxoheptan-3-yl)-2-(3-((4,4-difluorocyclohexyl)methyl)-3-methylureido)benzamide

To a vial containing 5-chloro-N-((2S,3S)-6,6-difluoro-2-hydroxy-1-(methylamino)-1-oxoheptan-3-yl)-2-(3-((4,4-difluorocyclohexyl)methyl)-3-methylureido)benzamide (57 mg, 0.103 mmol) was added Dess-MartinPeriodinane (95 mg, 0.224 mmol) & sodium bicarbonate (40 mg, 0.476 mmol) & finally DCM (5 mL). The reaction mixture was then capped and stirred at room temperature. Followed by LC/MS. After 1 hr the reaction mixture was quenched/diluted with 4 mL of saturated sodium thiosulfate followed by 1 mL water, then diluted with ~10 mL EtOAc, then stirred for ~15 minutes at room temperature. The reaction mixture was then suspended in EtOAc, washed with saturated sodium thiosulfate, then water, then brine. The organic layer was then dried over anhydrous sodium sulfate, filtered & concentrated. The resulting residue was then dissolved in DCM & purified by silica gel chromatography (0-80% EtOAc/Hex; 14 CV; 40 g ISCO). The desired fractions were concentrated to yield the title compound. LRMS m/z: (M+H)⁺ calculated 551.9; found 551.5.

¹H NMR (500 MHz, DMSO) δ 10.56 (s, 1H), 9.21 (d, J=6.8 Hz, 1H), 8.70 (d, J=4.8 Hz, 1H), 8.38 (d, J=9.1 Hz, 1H), 7.87 (d, J=2.5 Hz, 1H), 7.58-7.46 (m, 1H), 5.17-5.09 (m, 1H), 3.24-3.12 (m, 2H), 2.93 (s, 3H), 2.67 (d, J=4.8 Hz, 3H), 2.12-1.92 (m, 5H), 1.86-1.55 (m, 8H), 1.25-1.11 (m, 3H).

The following examples were prepared according to similar methods to those described above.

| Ex- | | | MS | MS |
|---|---|---|---|---|
| ample | Structure | Name | Calculated | Observed |
| 8 | 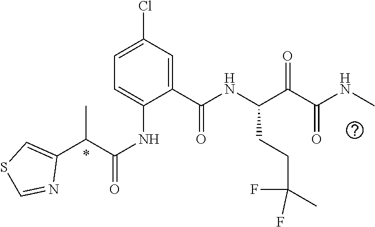 [SMILES: CNC(=O)C(=O)[C@H](CCC(C)(F)F)NC(=O)c1cc(Cl)ccc1NC(=O)C(C)c1cscn1] | 5-chloro-N-[(1S)-4,4-difluoro- 1-[2-(methylamino)-2-oxo- acetyl]pentyl]-2-(2-thiazol-4- ylpropanoylamino)benzamide | 501.9465 | 501.1182 |
| 9 | 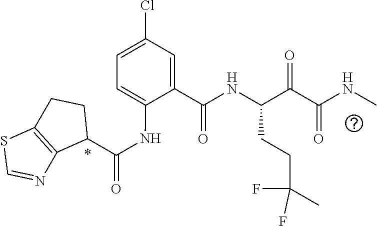 [SMILES: CNC(=O)C(=O)[C@H](CCC(C)(F)F)NC(=O)c1cc(Cl)ccc1NC(=O)C1CCc2scnc21] | N-[4-chloro-2-[[(1S)-4,4- difluoro-1-[2-(methylamino)-2- oxo-acetyl] pentyl]carbamoyl]phenyl]- 5,6-dihydro-4H- cyclopenta[d]thiazole-4- carboxamide | 513.9572 | 513.118 |
| 10 | 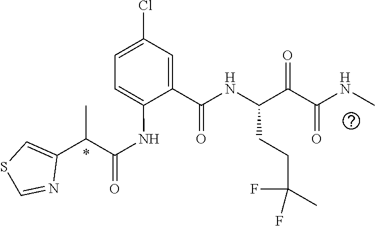 [SMILES: CNC(=O)C(=O)[C@H](CCC(C)(F)F)NC(=O)c1cc(Cl)ccc1NC(=O)C(C)c1cscn1] | 5-chloro-N-[(1S)-4,4-difluoro- 1-[2-(methylamino)-2-oxo- acetyl]pentyl]-2-(2-thiazol-4- ylpropanoylamino)benzamide | 501.9465 | 501.1183 |
| 11 | 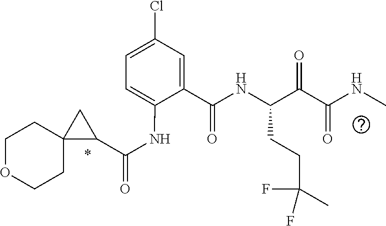 [SMILES: CNC(=O)C(=O)[C@H](CCC(C)(F)F)NC(=O)c1cc(Cl)ccc1NC(=O)C1CC12CCOCC2] | N-[4-chloro-2-[[(1S)-4,4- difluoro-1-[2-(methylamino)-2- oxo-acetyl] pentyl]carbamoyl]phenyl]- 6-oxaspiro[2.5]octane-2- carboxamide | 500.9353 | 500.1773 |
| 12 | 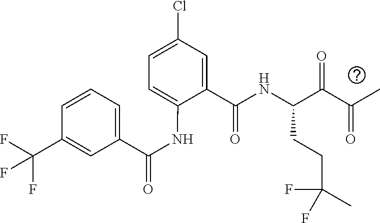 [SMILES: CC(=O)C(=O)[C@H](CCC(C)(F)F)NC(=O)c1cc(Cl)ccc1NC(=O)c1cccc(C(F)(F)F)c1] | 5-chloro-N-[(1S)-1-[2- (cyclopropylamino)-2-oxo- acetyl]-4.4-difluoro-pentyl]-2- [[3- (trifluoromethyl)benzoyl] amino]benzamide | 560.9128 | 560.1378 |
| 13 | 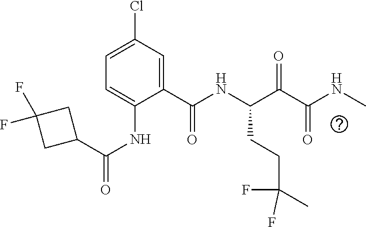 [SMILES: CNC(=O)C(=O)[C@H](CCC(C)(F)F)NC(=O)c1cc(Cl)ccc1NC(=O)C1CC(F)(F)C1] | 5-chloro-2-[(3,3- difluorocyclobutanecarbonyl) amino]-N-[(1S)-4,4-difluoro-1- [2-(methylamino)-2-oxo- acetyl]pentyl]benzamide | 480.853 | 480.1311 |
| 14 | 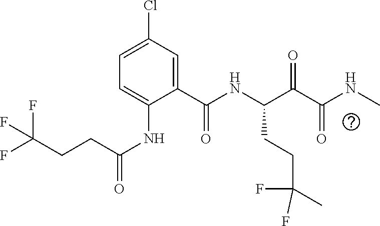 [SMILES: CNC(=O)C(=O)[C@H](CCC(C)(F)F)NC(=O)c1cc(Cl)ccc1NC(=O)CCC(F)(F)F] | 5-chloro-N-[(1S)-4,4-difluoro- 1-[2-(methylamino)-2-oxo- acetyl]pentyl]-2-(4,4,4- trifluorobutanoylamino) benzamide | 486.8328 | 486.1228 |
| 15 | 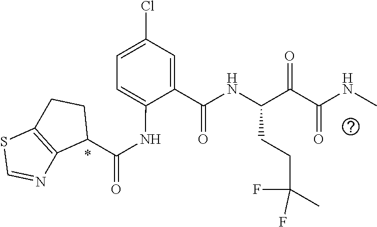 [SMILES: CNC(=O)C(=O)[C@H](CCC(C)(F)F)NC(=O)c1cc(Cl)ccc1NC(=O)C1CCc2scnc21] | N-[4-chloro-2-[[(1S)-4,4- difluoro-1-[2-(methylamino)-2- oxo-acetyl] pentyl]carbamoyl]phenyl]- 5,6-dihydro-4H- cyclopenta[d]thiazole-4- carboxamide | 513.9572 | 513.1177 |
| 16 | 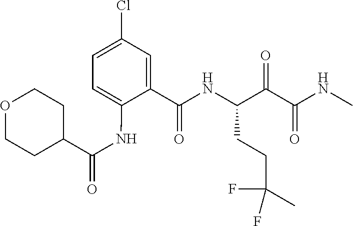 [SMILES: CNC(=O)C(=O)[C@H](CCC(C)(F)F)NC(=O)c1cc(Cl)ccc1NC(=O)C1CCOCC1] | N-[4-chloro-2-[[(1S)-4,4- difluoro-1-[2-(methylamino)-2- oxo-acetyl] pentyl]carbamoyl]phenyl] tetrahydropyran-4- carboxamide | 474.8981 | 474.1616 |
| 17 | 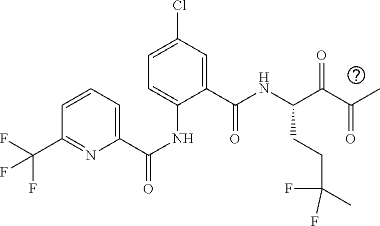 [SMILES: CC(=O)C(=O)[C@H](CCC(C)(F)F)NC(=O)c1cc(Cl)ccc1NC(=O)c1cccc(C(F)(F)F)n1] | N-[4-chloro-2-[[(1S)-4,4- difluoro-1-[2-(methylamino)-2- oxo-acetyl] pentyl]carbamoyl]phenyl]- 6-(trifluoromethyl)pyridine-2- carboxamide | 535.8636 | 535.118 |
| 18 | 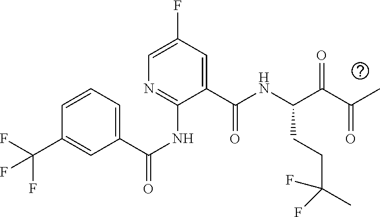 [SMILES: CC(=O)C(=O)[C@H](CCC(C)(F)F)NC(=O)c1cc(F)cnc1NC(=O)c1cccc(C(F)(F)F)c1] | N-[(1S)-4,4-difluoro-1-[2- (methylamino)-2-oxo- acetyl]pentyl]-5-fluoro-2-[[3- (trifluoromethyl)benzoyl] amino]pyridine-3-carboxamide | 519.409 | 519.1469 |
| 19 | 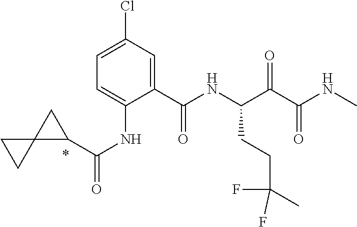 [SMILES: CNC(=O)C(=O)[C@H](CCC(C)(F)F)NC(=O)c1cc(Cl)ccc1NC(=O)C1CC12CC2] | (2S)-N-[4-chloro-2-[[(1S)-4,4- difluoro-1-[2-(methylamino)-2- oxo-acetyl] pentyl]carbamoyl]phenyl] spiro[2.2]pentane-2- carboxamide | 456.8828 | 456.1509 |
| 20 | 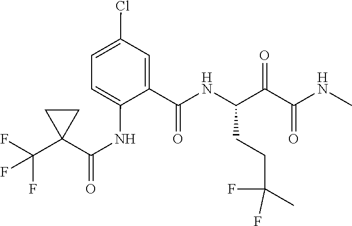 [SMILES: CNC(=O)C(=O)[C@H](CCC(C)(F)F)NC(=O)c1cc(Cl)ccc1NC(=O)C1(C(F)(F)F)CC1] | 5-chloro-N-[(1S)-4,4-difluoro- 1-[2-(methylamino)-2-oxo- acetyl]pentyl]-2-[[1- (trifluoromethyl)cyclopropane- carbonyl]amino]benzamide | 498.8435 | 498.1223 |
| 21 | 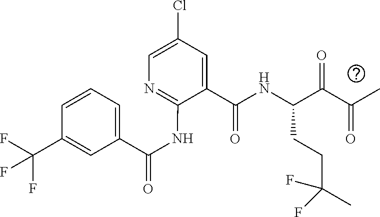 [SMILES: CC(=O)C(=O)[C@H](CCC(C)(F)F)NC(=O)c1cc(Cl)cnc1NC(=O)c1cccc(C(F)(F)F)c1] | 5-chloro-N-[(1S)-4,4-difluoro- 1-[2-(methylamino)-2-oxo- acetyl]pentyl]-2-[[3- (trifluoromethyl)benzoyl] amino]pyridine-3-carboxamide | 535.8636 | 535.1185 |
| 22 | 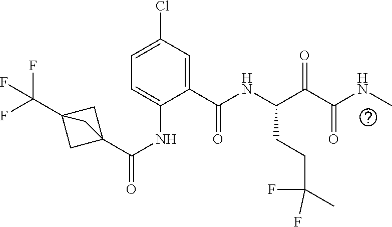 [SMILES: CNC(=O)C(=O)[C@H](CCC(C)(F)F)NC(=O)c1cc(Cl)ccc1NC(=O)C12CC(C(F)(F)F)(C1)C2] | N-[4-chloro-2-[[(1S)-4,4- difluoro-1-[2-(methylamino)-2- oxo- acetyl]pentyl]carbamoyl] phenyl]-3- (trifluoromethyl)bicyclo[1.1.1] pentane-1-carboxamide | 524.8807 | 524.1383 |
| 23 | 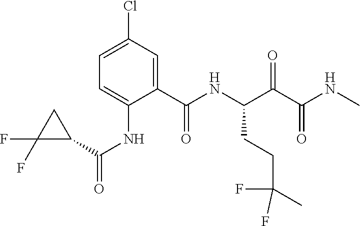 [SMILES: CNC(=O)C(=O)[C@H](CCC(C)(F)F)NC(=O)c1cc(Cl)ccc1NC(=O)[C@H]1CC1(F)F] | 5-chloro-2-[[(1R)-2,2- difluorocyclopropanecarbonyl] amino]-N-[(1S)-4,4-difluoro-1- [2-(methylamino)-2-oxo- acetyl]pentyl]benzamide | 466.8264 | 466.1162 |
| 24 | 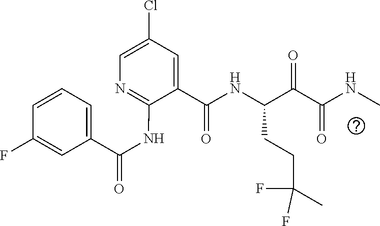 [SMILES: CNC(=O)C(=O)[C@H](CCC(C)(F)F)NC(=O)c1cc(Cl)cnc1NC(=O)c1cccc(F)c1] | 5-chloro-N-[(1S)-4,4-difluoro- 1-[2-(methylamino)-2-oxo- acetyl]pentyl]-2-[(3- fluorobenzoyl)amino]pyridine- 3-carboxamide | 485.8561 | 485.1212 |
| 25 | 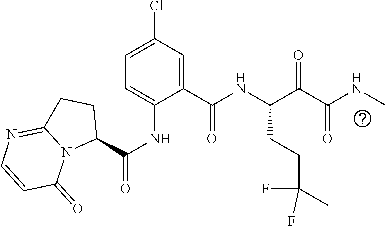 [SMILES: CNC(=O)C(=O)[C@H](CCC(C)(F)F)NC(=O)c1cc(Cl)ccc1NC(=O)[C@@H]1CCc2nccc(=O)n21] | (6S)-N-[4-chloro-2-[[(1S)-4,4- difluoro-1-[2-(methylamino)-2- oxo-acetyl] pentyl]carbamoyl]phenyl]- 4-oxo-7,8-dihydro-6H- pyrrolo[1,2-a]pyrimidine-6- carboxamide | 524.917 | 524.1515 |
| 26 | 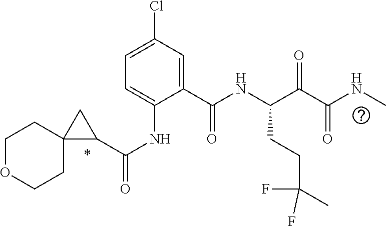 [SMILES: CNC(=O)C(=O)[C@H](CCC(C)(F)F)NC(=O)c1cc(Cl)ccc1NC(=O)C1CC12CCOCC2] | N-[4-chloro-2-[[(1S)-4,4- difluoro-1-[2-(methylamino)-2- oxo-acetyl] pentyl]carbamoyl]phenyl]- 6-oxaspiro[2.5]octane-2- carboxamide | 500.9353 | 500.1773 |
| 27 | 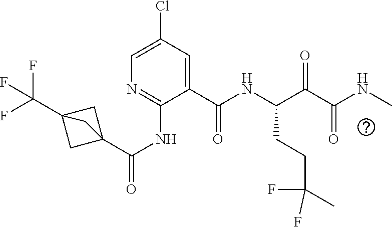 [SMILES: CNC(=O)C(=O)[C@H](CCC(C)(F)F)NC(=O)c1cc(Cl)cnc1NC(=O)C12CC(C(F)(F)F)(C1)C2] | 5-chloro-N-[(1S)-1-[2- (cyclopropylamino)-2-oxo- acetyl]-4,4-difluoro-pentyl]-2- [[3- (trifluoromethyl)bicyclo[1.1.1] pentane-1- carbonyl]amino]pyridine-3- carboxamide | 551.9061 | 551.1496 |
| 28 | 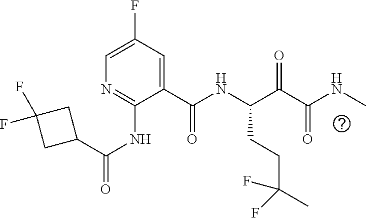 [SMILES: CNC(=O)C(=O)[C@H](CCC(C)(F)F)NC(=O)c1cc(F)cnc1NC(=O)C1CC(F)(F)C1] | 2-[(3,3- difluorocyclobutanecarbonyl) amino]-N-[(1S)-4,4-difluoro-1- [2-(methylamino)-2-oxo- acetyl]pentyl]-5-fluoro- pyridine-3-carboxamide | 465.3865 | 465.1562 |
| 29 | 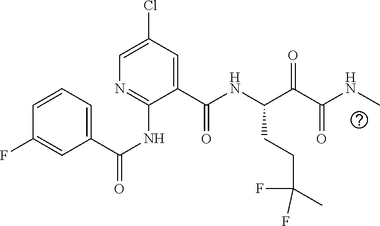 [SMILES: CNC(=O)C(=O)[C@H](CCC(C)(F)F)NC(=O)c1cc(Cl)cnc1NC(=O)c1cccc(F)c1] | 5-chloro-N-[(1S)-1-[2- (cyclopropylamino)-2-oxo- acetyl]-4,4-difluoro-pentyl]-2- [(3- fluorobenzoyl)amino]pyridine- 3-carboxamide | 511.8934 | 511.1365 |
| 30 | 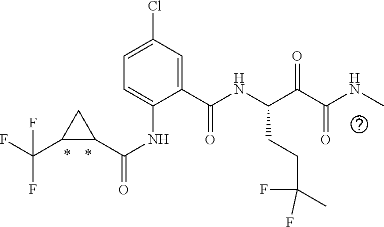 [SMILES: CNC(=O)C(=O)[C@H](CCC(C)(F)F)NC(=O)c1cc(Cl)ccc1NC(=O)C1CC1C(F)(F)F] | 5-chloro-N-[(1S)-4,4-difluoro- 1-[2-(methylamino)-2-oxo- acctyl]pentyl]-2-[[2- (trifluoromethyl)cyclopropane- carbonyl]amino]benzamide | 498.8435 | 498.1213 |
| 31 | 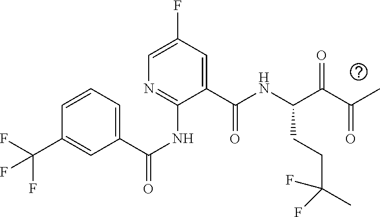 [SMILES: CC(=O)C(=O)[C@H](CCC(C)(F)F)NC(=O)c1cc(F)cnc1NC(=O)c1cccc(C(F)(F)F)c1] | N-[(1S)-1-[2- (cyclopropylamino)-2-oxo- acetyl]-4,4-difluoro-pentyl]-5- fluoro-2-[[3- (trifluoromethyl)benzoyl]amino] pyridine-3-carboxamide | 545.4463 | 545.1629 |
| 32 | 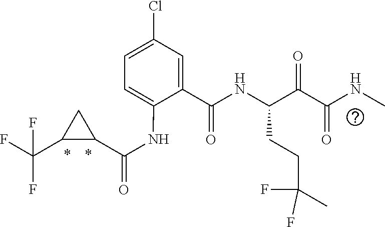 [SMILES: CNC(=O)C(=O)[C@H](CCC(C)(F)F)NC(=O)c1cc(Cl)ccc1NC(=O)C1CC1C(F)(F)F] | 5-chloro-N-[(1S)-4,4-difluoro- 1-[2-(methylamino)-2-oxo- acetyl]pentyl]-2-[[2- (trifluoromethyl)cyclopropane- carbonyl]amino]benzamide | 498.8435 | 498.1221 |
| 33 | 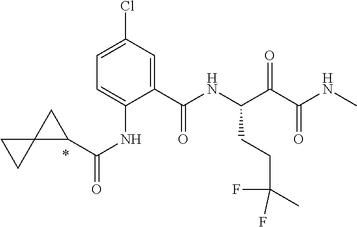 [SMILES: CNC(=O)C(=O)[C@H](CCC(C)(F)F)NC(=O)c1cc(Cl)ccc1NC(=O)C1CC12CC2] | (2R)-N-[4-chloro-2-[[(1S)-4,4- difluoro-1-[2-(methylamino)-2- oxo-acetyl] pentyl]carbamoyl]phenyl] spiro[2.2]pentane-2- carboxamide | 456.8828 | 456.1504 |
| 34 | 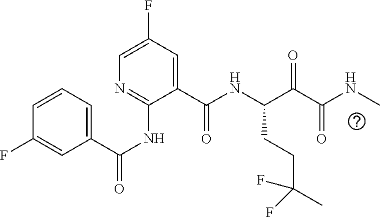 [SMILES: CNC(=O)C(=O)[C@H](CCC(C)(F)F)NC(=O)c1cc(F)cnc1NC(=O)c1cccc(F)c1] | N-[(1S)-4,4-difluoro-1-[2- (methylamino)-2-oxo- acetyl]pentyl]-5-fluoro-2-[(3- fluorobenzoyl)amino]pyridine- 3-carboxamide | 469.4015 | 469.1507 |
| 35 | 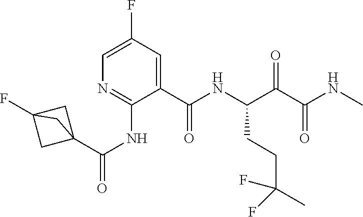 [SMILES: CNC(=O)C(=O)[C@H](CCC(C)(F)F)NC(=O)c1cc(F)cnc1NC(=O)C12CC(F)(C1)C2] | N-[(1S)-4,4-difluoro-1-[2- (methylamino)-2-oxo- acetyl]pentyl]-5-fluoro-2-[(3- fluorobicyclo[1.1.1]pentane-1- carbonyl)amino]pyridine-3- carboxamide | 459.4067 | 459.1667 |
| 36 | 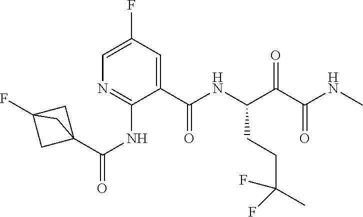 [SMILES: CNC(=O)C(=O)[C@H](CCC(C)(F)F)NC(=O)c1cc(F)cnc1NC(=O)C12CC(F)(C1)C2] | 5-chloro-N-[(1S)-4,4-difluoro- 1-[2-(methylamino)-2-oxo- acetyl]pentyl]-2-[[1- (trifluoromethyl)cyclopropane- carbonyl]amino]pyridine-3- carboxamide | 499.8315 | 499.1173 |
| 37 | 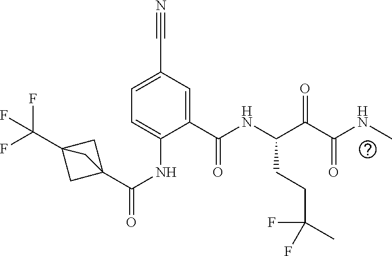 [SMILES: CNC(=O)C(=O)[C@H](CCC(C)(F)F)NC(=O)c1cc(C#N)ccc1NC(=O)C12CC(C(F)(F)F)(C1)C2] | N-[4-cyano-2-[[(1S)-4,4- difluoro-1-[2-(methylamino)-2- oxo- acetyl]pentyl]carbamoyl] phenyl]-3- (trifluoromethyl)bicyclo[1.1.1] pentane-1-carboxamide | 515.4451 | 515.1719 |
| 38 | 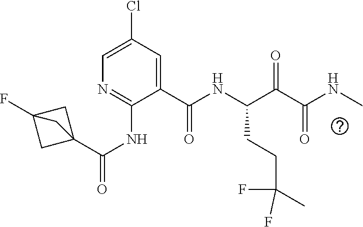 [SMILES: CNC(=O)C(=O)[C@H](CCC(C)(F)F)NC(=O)c1cc(Cl)cnc1NC(=O)C12CC(F)(C1)C2] | 5-chloro-N-[(1S)-4,4-difluoro- 1-[2-(methylamino)-2-oxo- acetyl]pentyl]-2-[(3- fluorobicyclo[1.1.1]pentane-1- carbonyl)amino]pyridine-3- carboxamide | 475.8613 | 475.137 |
| 39 | 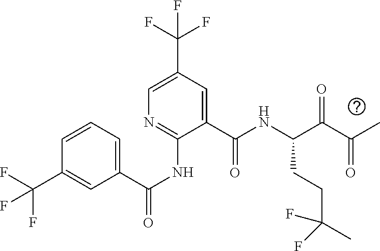 [SMILES: CC(=O)C(=O)[C@H](CCC(C)(F)F)NC(=O)c1cc(C(F)(F)F)cnc1NC(=O)c1cccc(C(F)(F)F)c1] | N-[(1S)-1-[2- (cyclopropylamino)-2-oxo- acetyl]-4,4-difluoro-pentyl]-5- (trifluoromethyl)-2-[[3- (trifluoromethyl)benzoyl] amino]pyridine-3-carboxamide | 595.4538 | 595.1597 |
| 40 | 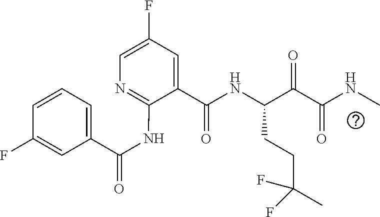 [SMILES: CNC(=O)C(=O)[C@H](CCC(C)(F)F)NC(=O)c1cc(F)cnc1NC(=O)c1cccc(F)c1] | N-[(1S)-1-[2- (cyclopropylamino)-2-oxo- acetyl]-4,4-difluoro-pentyl]-5- fluoro-2-[(3- fluorobenzoyl)amino]pyridine- 3-carboxamide | 495.4388 | 495.166 |
| 41 | 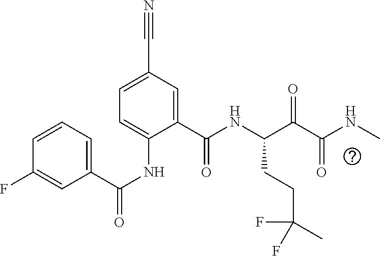 [SMILES: CNC(=O)C(=O)[C@H](CCC(C)(F)F)NC(=O)c1cc(C#N)ccc1NC(=O)c1cccc(F)c1] | 5-cyano-N-[(1S)-4,4-difluoro- 1-[2-(methylamino)-2-oxo- acetyl]pentyl]-2-[(3- fluorobenzoyl)amino]benzamide | 475.4325 | 475.1601 |
| 42 | 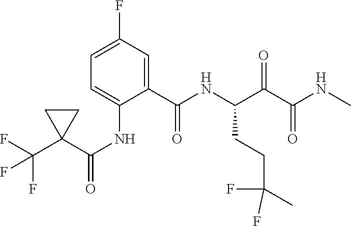 [SMILES: CNC(=O)C(=O)[C@H](CCC(C)(F)F)NC(=O)c1cc(F)ccc1NC(=O)C1(C(F)(F)F)CC1] | N-[(1S)-4,4-difluoro-1-[2- (methylamino)-2-oxo- acetyl]pentyl]-5-fluoro-2-[[1- (trifluoromethyl)cyclopropane- carbonyl]amino]benzamide | 482.3889 | 482.1516 |
| 43 | 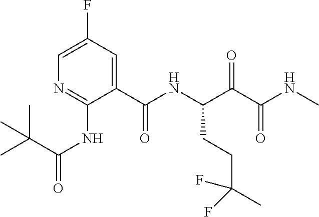 [SMILES: CNC(=O)C(=O)[C@H](CCC(C)(F)F)NC(=O)c1cc(F)cnc1NC(=O)C(C)(C)C] | N-[(1S)-4,4-difluoro-1-[2- (methylamino)-2-oxo- acetyl]pentyl]-2-(2,2- dimethylpropanoylamino)-5- fluoro-pyridine-3-carboxamide | 431.4214 | 431.1916 |
| 44 | 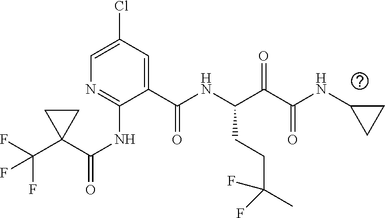 [SMILES: CC(F)(F)CC[C@H](NC(=O)c1cc(Cl)cnc1NC(=O)C1(C(F)(F)F)CC1)C(=O)C(=O)NC1CC1] | 5-chloro-N-[(1S)-1-[2- (cyclopropylamino)-2-oxo- acetyl]-4,4-difluoro-pentyl]-2- [[1- (trifluoromethyl)cyclopropane- carbonyl]amino]pyridine-3- carboxamide | 525.8688 | 525.133 |
| 45 | 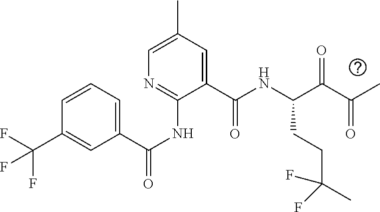 [SMILES: CC(=O)C(=O)[C@H](CCC(C)(F)F)NC(=O)c1cc(C)cnc1NC(=O)c1cccc(C(F)(F)F)c1] | N-[(1S)-1-[2- (cyclopropylamino)-2-oxo- acetyl]-4,4-difluoro-pentyl]-5- methyl-2-[[3- (trifluoromethyl)benzoyl] amino]pyridine-3-carboxamide | 541.4824 | 541.1891 |
| 46 | 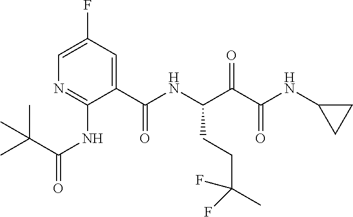 [SMILES: CC(F)(F)CC[C@H](NC(=O)c1cc(F)cnc1NC(=O)C(C)(C)C)C(=O)C(=O)NC1CC1] | N-[(1S)-1-[2- (cyclopropylamino)-2-oxo- acetyl]-4,4-difluoro-pentyl]-2- (2,2-dimethylpropanoylamino)- 5-fluoro-pyridine-3- carboxamide | 457.4587 | 457.2074 |
| 47 | 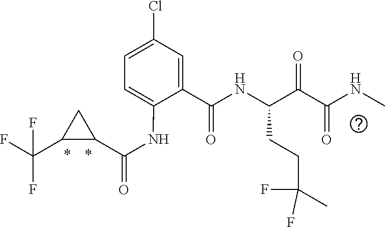 [SMILES: CNC(=O)C(=O)[C@H](CCC(C)(F)F)NC(=O)c1cc(Cl)ccc1NC(=O)C1CC1C(F)(F)F] | 5-chloro-N-[(1S)-4,4-difluoro- 1-[2-(methylamino)-2-oxo- acetyl]pentyl]-2-[[2- (trifluoromethyl)cyclopropane- carbonyl]amino]benzamide | 498.8435 | 498.122 |
| 48 | 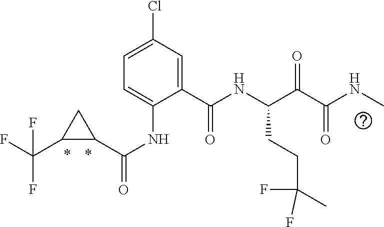 [SMILES: CNC(=O)C(=O)[C@H](CCC(C)(F)F)NC(=O)c1cc(Cl)ccc1NC(=O)C1CC1C(F)(F)F] | 5-chloro-N-[(1S)-4,4-difluoro- 1-[2-(methylamino)-2-oxo- acetyl]pentyl]-2-[[2- (trifluoromethyl)cyclopropane- carbonyl]amino]benzamide | 498.8435 | 498.1229 |
| 49 | 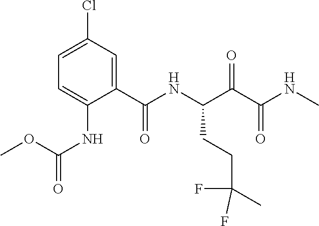 | methyl N-[4-chloro-2-[[(1S)- 4,4-difluoro-1-[2- (methylamino)-2-oxo- acetyl]pentyl]carbamoyl] phenyl]carbamate | 420.8076 | 420.1138 |
| 50 | 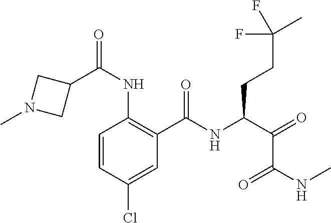 | N-[4-chloro-2-[[(1S)-4,4- difluoro-1-[2-(methylamino)-2- oxo-acetyl] pentyl]carbamoyl]phenyl]- 1-methyl-azetidine-3- carboxamide | 459.8867 | 459.1617 |
| 51 | 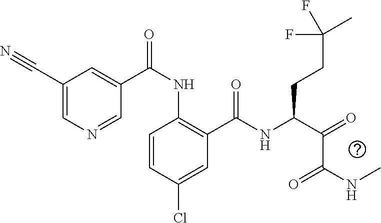 [SMILES: CNC(=O)C(=O)[C@H](CCC(C)(F)F)NC(=O)c1cc(Cl)ccc1NC(=O)c1cncc(C#N)c1] | N-[4-chloro-2-[[(1S)-4,4- difluoro-1-[2-(methylamino)-2- oxo-acetyl] pentyl]carbamoyl]phenyl]- 5-cyano-pyridine-3- carboxamide | 492.8751 | 492.1245 |
| 52 | 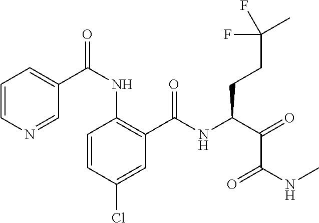 [SMILES: CNC(=O)C(=O)[C@H](CCC(C)(F)F)NC(=O)c1cc(Cl)ccc1NC(=O)c1cccnc1] | N-[4-chloro-2-[(1S)-4,4- difluoro-1-[2-(methylamino)-2- oxo-acetyl] pentyl]carbamoyl]phenyl] pyridine-3-carboxamide | 467.8657 | 467.1304 |
| 53 | 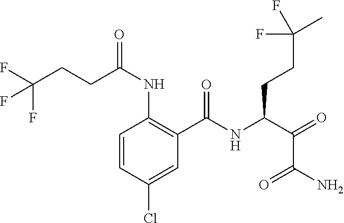 [SMILES: CC(F)(F)CC[C@H](NC(=O)c1cc(Cl)ccc1NC(=O)CCC(F)(F)F)C(=O)C(N)=O] | 5-chloro-N-[(1S)-4,4-difluoro- 1-oxamoyl-pentyl]-2-(4,4,4- trifluorobutanoylamino) benzamide | 472.8062 | 472.1059 |
| 54 | 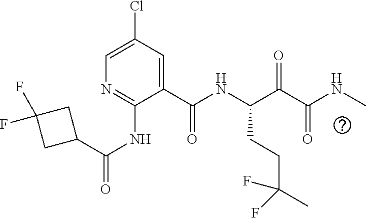 [SMILES: CNC(=O)C(=O)[C@H](CCC(C)(F)F)NC(=O)c1cc(Cl)cnc1NC(=O)C1CC(F)(F)C1] | 5-chloro-2-[(3,3- difluorocyclobutanecarbonyl) amino]-N-[(1S)-4,4-difluoro-1- [2-(methylamino)-2-oxo- acetyl]pentyl]pyridine-3- carboxamide | 481.8411 | 481.1265 |
| 55 | 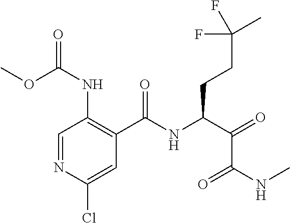 | methyl N-[6-chloro-4-[[(1S)- 4,4-difluoro-1-[2- (methylamino)-2-oxo- acetyl]pentyl]carbamoyl]-3- pyridyl]carbamate | 421.7957 | 421.1087 |
| 56 | 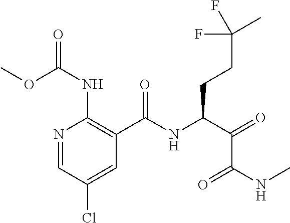 | methyl N-[5-chloro-3-[[(1S)- 4,4-difluoro-1-[2- (methylamino)-2-oxo- acetyl]pentyl]carbamoyl]-2- pyridyl]carbamate | 421.7957 | 421.109 |
| 58 | 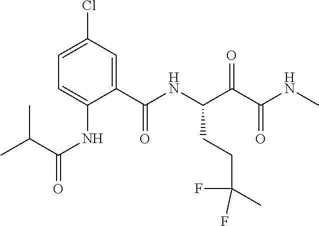 [SMILES: CNC(=O)C(=O)[C@H](CCC(C)(F)F)NC(=O)c1cc(Cl)ccc1NC(=O)C(C)C] | 5-chloro-N-[(1S)-4,4-difluoro- 1-[2-(methylamino)-2-oxo- acetyl]pentyl]-2-(2- methylpropanoylamino) benzamide | 430.1345 | 430.1355 |
| 59 | 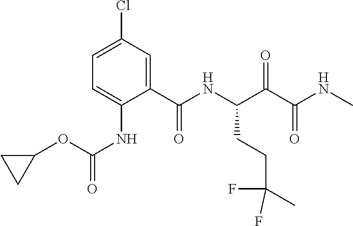 [SMILES: CNC(=O)C(=O)[C@H](CCC(C)(F)F)NC(=O)c1cc(Cl)ccc1NC(=O)OC1CC1] | cyclopropyl N-[4-chloro-2- [[(1S)-4,4-difluoro-1-[2- (mcthylamino)-2-oxo- acetyl]pentyl]carbamoyl] phenyl]carbamate | 446.84489 | 446.4 |
| 60 | 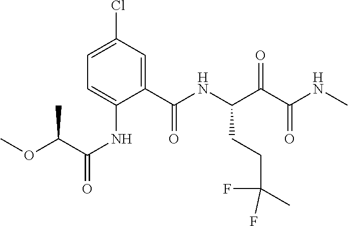 | 5-chloro-N-[(1S)-4,4-difluoro- 1-[2-(methylamino)-2-oxo- acetyl]pentyl]-2-[[(2S)-2- methoxypropanoyl]amino] benzamide | 446.1295 | 446.13 |

* denotes a chiral center where the stereochemistry is unidentified

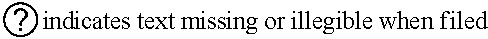

### SARS2 Coronavirus 3CL Protease Assay

The enzymatic activity of SARS2 coronavirus 3CL protease was determined in a FRET (fluorescence resonance energy transfer)-based assay measuring the cleavage of a peptide substrate by recombinantly expressed and purified enzyme. Cleavage of the peptide SEQ ID NO: 1 (CPC Scientific) by SARS2 3CL protease was measured in reaction buffer (50 mM Hepes pH 7.5, 0.01% Triton X-100, 0.01% BSA, 2 mM DTT). SARS2 3CL protease (5 nM final concentration) was pre-incubated with compound for 30 minutes before reaction initiation with peptide substrate (15 uM final concentration). Room temperature reactions (4 h) were quenched by addition of a high dose of inhibitor and read on an appropriate plate reader (excitation wavelength=495 nm, emission wavelength=520 nm). Data were analyzed by a standard 4 parameter fit to determine IC₅₀ values.

The compounds of the instant invention were tested in the assay described above and the results appear in the table below.

TABLE I
IC₅₀ values (nM) for Examples in the SARS2 3CLPro FRET assay

| Example | FRET Potency (nM) |
|---|---|
| 1 | 8.1 |
| 2 | 11.6 |
| 3 | 24.0 |
| 4 | 9.9 |
| 5 | 9.2 |
| 6A | 25.8 |
| 6B | 154.5 |
| 6C | 13.6 |
| 6D | 702.1 |
| 7 | 18.0 |
| 8 | 6.6 |
| 9 | 7.0 |
| 10 | 7.0 |
| 11 | 7.7 |
| 12 | 7.9 |
| 13 | 8.0 |
| 14 | 8.2 |
| 15 | 8.4 |
| 16 | 8.5 |
| 17 | 8.7 |
| 18 | 9.8 |
| 19 | 10.5 |
| 20 | 11.6 |
| 21 | 11.7 |
| 22 | 13.0 |
| 23 | 13.2 |
| 24 | 14.0 |
| 25 | 14.8 |
| 26 | 15.1 |
| 27 | 17.4 |
| 28 | 19.8 |
| 29 | 20.4 |
| 30 | 20.7 |
| 31 | 20.9 |
| 32 | 21.3 |
| 33 | 21.5 |
| 34 | 23.8 |
| 35 | 23.9 |
| 36 | 24.4 |
| 37 | 31.1 |
| 38 | 40.4 |
| 39 | 40.9 |
| 40 | 41.6 |
| 41 | 45.3 |
| 42 | 47.0 |
| 43 | 49.6 |
| 44 | 50.4 |
| 45 | 76.2 |
| 46 | 94.8 |
| 47 | 20.5 |
| 48 | 11.3 |
| 49 | 7.1 |
| 50 | 21.8 |
| 51 | 15.1 |
| 52 | 15.6 |
| 53 | 97.5 |
| 54 | 7.5 |
| 55 | 93.8 |
| 56 | 4.2 |
| 57 | 6.0 |
| 58 | 6.2 |
| 59 | 13.1 |
| 60 | 9.4 |

1. A compound of formula I 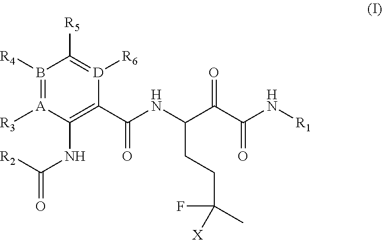
or a pharmaceutically acceptable salt thereof, wherein:
R₁ is H, (C₃-C₆)cycloalkyl, (C₁-C₆)alkyl, (C₁-C₆)alkyl-OH, phenyl, (C₁-C₆)alkyl-phenyl, (C₄-C₆) heterocycloalkyl containing 1 to 3 heteroatom(s) independently selected from N, O, or S, (C₁-C₆)alkyl-(C₄-C₆) heterocycloalkyl containing 1 to 3 heteroatom(s) independently selected from N, O, or S, (C₁-C₆)alkyl-(C₅-C₆) heteroaryl containing 1 to 3 heteroatom(s) independently selected from N, O, or S, (C₅-C₆) heteroaryl containing 1 to 3 heteroatom(s) independently selected from N, O, or S;
R₂ is (C₁-C₆)alkyl; (C₁-C₆)alkyl-CF₃; (C₁-C₆)alkyl-OH; (C₁-C₆)alkyl-O—CH₃; (C₁-C₆)alkyl-O—CF₃; (C₁-C₆)alkyl-O—(C₃-C₁₀)cycloalkyl optionally substituted by an OH, —(C₁-C₆)alkyl, CF₃, —(C₁-C₆)alkyl-CF₃, or up to 3 halogen; (CF₂)-phenyl; (C₁-C₆)alkyl-phenyl; (C₃-C₁₀)cycloalkyl optionally substituted by an OH, —(C₁-C₆)alkyl, CF₃, —(C₁-C₆)alkyl-CF₃, or up to 3 halogen; (C₁-C₆)alkyl-(C₃-C₁₀)cycloalkyl optionally substituted by a OH, —(C₁-C₆)alkyl, CF₃, —(C₁-C₆)alkyl-CF₃, or up to 3 halogen; (C₄-C₁₀) heterocycle including up to 3 heteroatoms independently selected from N, O, and S and optionally substituted by a carbonyl, OH, —(C₁-C₆)alkyl, CF₃, —(C₁-C₆)alkyl-CF₃, or up to 3 halogen; (C₁-C₆)alkyl-(C₄-C₁₀) heterocycle including up to 3 heteroatoms independently selected from N, O, and S and optionally substituted by a carbonyl, OH, —(C₁-C₆)alkyl, CF₃, —(C₁-C₆)alkyl-CF₃, or up to 3 halogen; (C₆-C₁₀) aryl optionally substituted by an OH, —(C₁-C₆)alkyl, CF₃, —(C₁-C₆)alkyl-CF₃, or up to 3 halogen; (C₁-C₆)alkyl-(C₆-C₁₀) aryl optionally substituted by an OH, —(C₁-C₆)alkyl, CF₃, —(C₁-C₆)alkyl-CF₃, or up to 3 halogen; (C₅-C₁₀) heteroaryl including up to 4 heteroatoms independently selected from N, O, and S and optionally substituted by an OH, CN, CHF₂, —(C₁-C₆)alkyl, CF₃, —(C₁-C₆)alkyl-CF₃, —(C₃-C₆)cycloalkyl, O—(C₁-C₆)alkyl, O—CF₃, O—(C₃-C₆)cycloalkyl, or up to 3 halogen; (C₁-C₆)alkyl-(C₅-C₁₀) heteroaryl including up to 4 heteroatoms independently selected from N, O, and S and optionally substituted by an OH, CN, CHF₂, (C₃-C₆)cycloalkyl, O—(C₃-C₆)cycloalkyl, —(C₁-C₆)alkyl, O—(C₁-C₆)alkyl, CF₃, O—CF₃, —(C₁-C₆)alkyl-CF₃, or up to 3 halogen; —O—(C₁-C₆)alkyl; —O—(C₁-C₆)alkyl-(C₃-C₆)cycloalkyl optionally substituted by an OH, CF₃, —(C₁-C₆)alkyl, —(C₁-C₆)alkyl-CF₃, or up to 3 halogen; —O—(C₃-C₆)cycloalkyl optionally substituted by an OH, CF₃, —(C₁-C₆)alkyl, —(C₁-C₆)alkyl-CF₃, or up to 3 halogen; (C₁-C₆)alkyl-N—CHO—O(C₁-C₆)alkyl; CHF₂; CF₃; or N(R₇)₂;
R₃ is H, F, Cl, or (C₁-C₆)alkyl;
R₄ is H, F, Cl, or (C₁-C₆)alkyl;
R₅ is H, F, Cl, CN, CF₃, O—CHF₂, O—CF₃, —(C₁-C₆)alkyl-CF₃, CHF₂, CF₃, (C₁-C₆)alkyl, (C₃-C₆)cycloalkyl, —O—(C₃-C₆)cycloalkyl, O—(C₁-C₆)alkyl;
R₆ is H, F, Cl, or (C₁-C₆)alkyl;
R₇ is independently H, (C₁-C₆)alkyl, (C₃-C₁₀)cycloalkyl optionally substituted by an OH, —(C₁-C₆)alkyl, CF₃, —(C₁-C₆)alkyl-CF₃, or up to 3 halogen; (C₁-C₆)alkyl-(C₃-C₁₀)cycloalkyl optionally substituted by an OH, —(C₁-C₆)alkyl, CF₃, —(C₁-C₆)alkyl-CF₃, or up to 3 halogen; (C₄-C₁₀) heterocycle including up to 3 heteroatoms selected from N, O, and S and optionally substituted by an OH, —(C₁-C₆)alkyl, CF₃, —(C₁-C₆)alkyl-CF₃, or up to 3 halogen; (C₁-C₆)alkyl-(C₄-C₁₀) heterocycle including up to 3 heteroatoms selected from N, O, and S and optionally substituted by an OH, —(C₁-C₆)alkyl, CF₃, —(C₁-C₆)alkyl-CF₃, or up to 3 halogen; (C₆-C₁₀) aryl optionally substituted by an OH, —(C₁-C₆)alkyl, CF₃, —(C₁-C₆)alkyl-CF₃, or up to 3 halogen; (C₁-C₆)alkyl-(C₆-C₁₀) aryl optionally substituted by an OH, —(C₁-C₆)alkyl, CF₃, —(C₁-C₆)alkyl-CF₃, or up to 3 halogen; (C₅-C₁₀) heteroaryl including up to 3 heteroatoms selected from N, O, and S and optionally substituted by an OH, —(C₁-C₆)alkyl, CF₃, —(C₁-C₆)alkyl-CF₃, or up to 3 halogen; (C₁-C₆)alkyl-(C₅-C₁₀) heteroaryl including up to 3 heteroatoms selected from N, O, and S and optionally substituted by an OH, —(C₁-C₆)alkyl, CF₃, —(C₁-C₆)alkyl-CF₃, or up to 3 halogen;
A is C or N;
B is C or N,
D is C or N, provided that only 1 of A, B, or D can be N and that if A, B, or D is N then the respective R₃, R₄, or R₆ is absent;
X is H or F.

2. The compound of claim 1 or a pharmaceutically acceptable salt thereof, wherein R₁ is H, (C₃-C₆)cycloalkyl, or (C₁-C₆)alkyl.

3. The compound of claim 1 or a pharmaceutically acceptable salt thereof, wherein R₁ is H, CH₃, or cyclopropyl.

4. The compound of claim 1 or a pharmaceutically acceptable salt thereof, wherein R₂ is selected from the group consisting of:
CH₃,

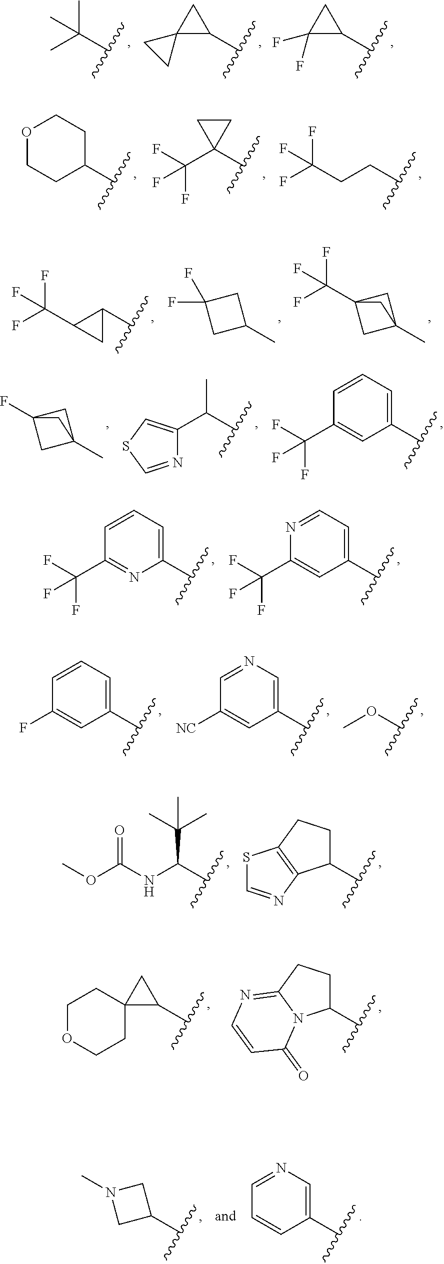

5. The compound of claim 1 or a pharmaceutically acceptable salt thereof, wherein
R₃ is H, F, or Cl.

6. The compound of claim 1 or a pharmaceutically acceptable salt thereof, wherein
R₅ is H, F, or Cl.

7. The compound of claim 1 or a pharmaceutically acceptable salt thereof, wherein R₅ is F, Cl, CHF₂, or CN.

8. The compound of claim 1 or a pharmaceutically acceptable salt thereof, wherein R₆ is H, F, or Cl.

9. The compound of claim 1 or a pharmaceutically acceptable salt thereof, wherein one of
A, B or D is N.

10. The compound of claim 1 or a pharmaceutically acceptable salt thereof, wherein
A, B, and D are all C.

11. The compound of claim 1 or a pharmaceutically acceptable salt thereof, wherein X is H.

12. The compound of claim 1 or a pharmaceutically acceptable salt thereof, wherein X is F.

13. The compound of claim 1 or a pharmaceutically acceptable salt thereof, wherein the compound is selected from the group consisting of:
N-[4-chloro-2-[[(1S)-4,4-difluoro-1-[2-(methylamino)-2-oxo-acetyl]pentyl]carbamoyl]phenyl]-2-(trifluoromethyl)pyridine-4-carboxamide;
5-chloro-N-[(1S)-4,4-difluoro-1-[2-(methylamino)-2-oxo-acetyl]pentyl]-2-[[3-(trifluoromethyl)bicyclo[1.1.1]pentane-1-carbonyl]amino]pyridine-3-carboxamide;
methyl N-[(1S)-1-[[4-chloro-2-[[(1S)-1-[2-(cyclopropylamino)-2-oxo-acetyl]-4,4-difluoro-pentyl]carbamoyl]phenyl]carbamoyl]-2,2-dimethyl-propyl]carbamate;
5-chloro-N-[(1S)-4,4-difluoro-1-[2-(methylamino)-2-oxo-acetyl]pentyl]-4-fluoro-2-(4,4,4-trifluorobutanoylamino)benzamide;
5-chloro-N-[(1S)-1-[2-(cyclopropylamino)-2-oxo-acetyl]-4,4-difluoro-pentyl]-2-[[3-(trifluoromethyl)benzoyl]amino]pyridine-3-carboxamide;
methyl N-[4-chloro-2-[[(1S)-4-fluoro-1-[2-(methylamino)-2-oxo-acetyl]pentyl]carbamoyl]phenyl]carbamate;
methyl N-[4-chloro-2-[[(1S)-4-fluoro-1-[2-(methylamino)-2-oxo-acetyl]pentyl]carbamoyl]phenyl]carbamate;
methyl N-[4-chloro-2-[[(1S)-4-fluoro-1-[2-(methylamino)-2-oxo-acetyl]pentyl]carbamoyl]phenyl]carbamate;
methyl N-[4-chloro-2-[[(1S)-4-fluoro-1-[2-(methylamino)-2-oxo-acetyl]pentyl]carbamoyl]phenyl]carbamate;
N-[4-chloro-2-[[(1S)-4,4-difluoro-1-oxamoyl-pentyl]carbamoyl]phenyl]-2-(trifluoromethyl)pyridine-4-carboxamide;
5-chloro-N-[(1S)-4,4-difluoro-1-[2-(methylamino)-2-oxo-acetyl]pentyl]-2-(2-thiazol-4-ylpropanoylamino)benzamide;
N-[4-chloro-2-[[(1S)-4,4-difluoro-1-[2-(methylamino)-2-oxo-acetyl]pentyl]carbamoyl]phenyl]-5,6-dihydro-4H-cyclopenta[d]thiazole-4-carboxamide;
5-chloro-N-[(1S)-4,4-difluoro-1-[2-(methylamino)-2-oxo-acetyl]pentyl]-2-(2-thiazol-4-ylpropanoylamino)benzamide;
N-[4-chloro-2-[[(1S)-4,4-difluoro-1-[2-(methylamino)-2-oxo-acetyl]pentyl]carbamoyl]phenyl]-6-oxaspiro[2.5]octane-2-carboxamide;
5-chloro-N-[(1S)-1-[2-(cyclopropylamino)-2-oxo-acetyl]-4,4-difluoro-pentyl]-2-[[3-(trifluoromethyl)benzoyl]amino]benzamide;
5-chloro-2-[(3,3-difluorocyclobutanecarbonyl)amino]-N-[(1S)-4,4-difluoro-1-[2-(methylamino)-2-oxo-acetyl]pentyl]benzamide;
5-chloro-N-[(1S)-4,4-difluoro-1-[2-(methylamino)-2-oxo-acetyl]pentyl]-2-(4,4,4-trifluorobutanoylamino)benzamide;
N-[4-chloro-2-[[(1S)-4,4-difluoro-1-[2-(methylamino)-2-oxo-acetyl]pentyl]carbamoyl]phenyl]-5,6-dihydro-4H-cyclopenta[d]thiazole-4-carboxamide;
N-[4-chloro-2-[[(1S)-4,4-difluoro-1-[2-(methylamino)-2-oxo-acetyl]pentyl]carbamoyl]phenyl]tetrahydropyran-4-carboxamide;
N-[4-chloro-2-[[(1S)-4,4-difluoro-1-[2-(methylamino)-2-oxo-acetyl]pentyl]carbamoyl]phenyl]-6-(trifluoromethyl)pyridine-2-carboxamide;
N-[(1S)-4,4-difluoro-1-[2-(methylamino)-2-oxo-acetyl]pentyl]-5-fluoro-2-[[3-(trifluoromethyl)benzoyl]amino]pyridine-3-carboxamide;
(2S)—N-[4-chloro-2-[[(1S)-4,4-difluoro-1-[2-(methylamino)-2-oxo-acetyl]pentyl]carbamoyl]phenyl]spiro[2.2]pentane-2-carboxamide;
5-chloro-N-[(1S)-4,4-difluoro-1-[2-(methylamino)-2-oxo-acetyl]pentyl]-2-[[1-(trifluoromethyl)cyclopropanecarbonyl]amino]benzamide;
5-chloro-N-[(1S)-4,4-difluoro-1-[2-(methylamino)-2-oxo-acetyl]pentyl]-2-[[3-(trifluoromethyl)benzoyl]amino]pyridine-3-carboxamide;
N-[4-chloro-2-[[(1S)-4,4-difluoro-1-[2-(methylamino)-2-oxo-acetyl]pentyl]carbamoyl]phenyl]-3-(trifluoromethyl)bicyclo[1.1.1]pentane-1-carboxamide;
5-chloro-2-[[(1R)-2,2-difluorocyclopropanecarbonyl]amino]-N-[(1S)-4,4-difluoro-1-[2-(methylamino)-2-oxo-acetyl]pentyl]benzamide;
5-chloro-N-[(1S)-4,4-difluoro-1-[2-(methylamino)-2-oxo-acetyl]pentyl]-2-[(3-fluorobenzoyl)amino]pyridine-3-carboxamide;
(6S)—N-[4-chloro-2-[[(1S)-4,4-difluoro-1-[2-(methylamino)-2-oxo-acetyl]pentyl]carbamoyl]phenyl]-4-oxo-7,8-dihydro-6H-pyrrolo[1,2-a]pyrimidine-6-carboxamide;
N-[4-chloro-2-[[(1S)-4,4-difluoro-1-[2-(methylamino)-2-oxo-acetyl]pentyl]carbamoyl]phenyl]-6-oxaspiro[2.5]octane-2-carboxamide;
5-chloro-N-[(1S)-1-[2-(cyclopropylamino)-2-oxo-acetyl]-4,4-difluoro-pentyl]-2-[[3-(trifluoromethyl)bicyclo[1.1.1]pentane-1-carbonyl]amino]pyridine-3-carboxamide;
2-[(3,3-difluorocyclobutanecarbonyl)amino]-N-[(1S)-4,4-difluoro-1-[2-(methylamino)-2-oxo-acetyl]pentyl]-5-fluoro-pyridine-3-carboxamide;
5-chloro-N-[(1S)-1-[2-(cyclopropylamino)-2-oxo-acetyl]-4,4-difluoro-pentyl]-2-[(3-fluorobenzoyl)amino]pyridine-3-carboxamide;
5-chloro-N-[(1S)-4,4-difluoro-1-[2-(methylamino)-2-oxo-acetyl]pentyl]-2-[[2-(trifluoromethyl)cyclopropanecarbonyl]amino]benzamide;
N-[(1S)-1-[2-(cyclopropylamino)-2-oxo-acetyl]-4,4-difluoro-pentyl]-5-fluoro-2-[[3-(trifluoromethyl)benzoyl]amino]pyridine-3-carboxamide;
5-chloro-N-[(1S)-4,4-difluoro-1-[2-(methylamino)-2-oxo-acetyl]pentyl]-2-[[2-(trifluoromethyl)cyclopropanecarbonyl]amino]benzamide;
(2R)—N-[4-chloro-2-[[(1S)-4,4-difluoro-1-[2-(methylamino)-2-oxo-acetyl]pentyl]carbamoyl]phenyl]spiro[2.2]pentane-2-carboxamide;
N-[(1S)-4,4-difluoro-1-[2-(methylamino)-2-oxo-acetyl]pentyl]-5-fluoro-2-[(3-fluorobenzoyl)amino]pyridine-3-carboxamide;
N-[(1S)-4,4-difluoro-1-[2-(methylamino)-2-oxo-acetyl]pentyl]-5-fluoro-2-[(3-fluorobicyclo[1.1.1]pentane-1-carbonyl)amino]pyridine-3-carboxamide;
5-chloro-N-[(1S)-4,4-difluoro-1-[2-(methylamino)-2-oxo-acetyl]pentyl]-2-[[1-(trifluoromethyl)cyclopropanecarbonyl]amino]pyridine-3-carboxamide;
N-[4-cyano-2-[[(1S)-4,4-difluoro-1-[2-(methylamino)-2-oxo-acetyl]pentyl]carbamoyl]phenyl]-3-(trifluoromethyl)bicyclo[1.1.1]pentane-1-carboxamide;
5-chloro-N-[(1S)-4,4-difluoro-1-[2-(methylamino)-2-oxo-acetyl]pentyl]-2-[(3-fluorobicyclo[1.1.1]pentane-1-carbonyl)amino]pyridine-3-carboxamide;
N-[(1S)-1-[2-(cyclopropylamino)-2-oxo-acetyl]-4,4-difluoro-pentyl]-5-(trifluoromethyl)-2-[[3-(trifluoromethyl)benzoyl]amino]pyridine-3-carboxamide;
N-[(1S)-1-[2-(cyclopropylamino)-2-oxo-acetyl]-4,4-difluoro-pentyl]-5-fluoro-2-[(3-fluorobenzoyl)amino]pyridine-3-carboxamide;
5-cyano-N-[(1S)-4,4-difluoro-1-[2-(methylamino)-2-oxo-acetyl]pentyl]-2-[(3-fluorobenzoyl)amino]benzamide;
N-[(1S)-4,4-difluoro-1-[2-(methylamino)-2-oxo-acetyl]pentyl]-5-fluoro-2-[[1-(trifluoromethyl)cyclopropanecarbonyl]amino]benzamide;
N-[(1S)-4,4-difluoro-1-[2-(methylamino)-2-oxo-acetyl]pentyl]-2-(2,2-dimethylpropanoylamino)-5-fluoro-pyridine-3-carboxamide;
5-chloro-N-[(1S)-1-[2-(cyclopropylamino)-2-oxo-acetyl]-4,4-difluoro-pentyl]-2-[[1-(trifluoromethyl)cyclopropanecarbonyl]amino]pyridine-3-carboxamide;
N-[(1S)-1-[2-(cyclopropylamino)-2-oxo-acetyl]-4,4-difluoro-pentyl]-5-methyl-2-[[3-(trifluoromethyl)benzoyl]amino]pyridine-3-carboxamide;
N-[(1S)-1-[2-(cyclopropylamino)-2-oxo-acetyl]-4,4-difluoro-pentyl]-2-(2,2-dimethylpropanoylamino)-5-fluoro-pyridine-3-carboxamide;
5-chloro-N-[(1S)-4,4-difluoro-1-[2-(methylamino)-2-oxo-acetyl]pentyl]-2-[[2-(trifluoromethyl)cyclopropanecarbonyl]amino]benzamide;
5-chloro-N-[(1S)-4,4-difluoro-1-[2-(methylamino)-2-oxo-acetyl]pentyl]-2-[[2-(trifluoromethyl)cyclopropanecarbonyl]amino]benzamide;
methyl N-[4-chloro-2-[[(1S)-4,4-difluoro-1-[2-(methylamino)-2-oxo-acetyl]pentyl]carbamoyl]phenyl]carbamate;
N-[4-chloro-2-[[(1S)-4,4-difluoro-1-[2-(methylamino)-2-oxo-acetyl]pentyl]carbamoyl]phenyl]-1-methyl-azetidine-3-carboxamide;
N-[4-chloro-2-[[(1S)-4,4-difluoro-1-[2-(methylamino)-2-oxo-acetyl]pentyl]carbamoyl]phenyl]-5-cyano-pyridine-3-carboxamide;
N-[4-chloro-2-[[(1S)-4,4-difluoro-1-[2-(methylamino)-2-oxo-acetyl]pentyl]carbamoyl]phenyl]pyridine-3-carboxamide;
5-chloro-N-[(1S)-4,4-difluoro-1-oxamoyl-pentyl]-2-(4,4,4-trifluorobutanoylamino)benzamide;
5-chloro-2-[(3,3-difluorocyclobutanecarbonyl)amino]-N-[(1S)-4,4-difluoro-1-[2-(methylamino)-2-oxo-acetyl]pentyl]pyridine-3-carboxamide;
methyl N-[6-chloro-4-[[(1S)-4,4-difluoro-1-[2-(methylamino)-2-oxo-acetyl]pentyl]carbamoyl]-3-pyridyl]carbamate;
methyl N-[5-chloro-3-[[(1S)-4,4-difluoro-1-[2-(methylamino)-2-oxo-acetyl]pentyl]carbamoyl]-2-pyridyl]carbamate;
5-chloro-N-[(1S)-4,4-difluoro-1-[2-(methylamino)-2-oxo-acetyl]pentyl]-2-(2-methylpropanoylamino)benzamide;
cyclopropyl N-[4-chloro-2-[[(1S)-4,4-difluoro-1-[2-(methylamino)-2-oxo-acetyl]pentyl]carbamoyl]phenyl]carbamate; and
5-chloro-N-[(1S)-4,4-difluoro-1-[2-(methylamino)-2-oxo-acetyl]pentyl]-2-[[(2S)-2-methoxypropanoyl]amino]benzamide.

14. The compound of claim 1 or a pharmaceutically acceptable salt thereof, wherein the compound is selected from the group consisting of:

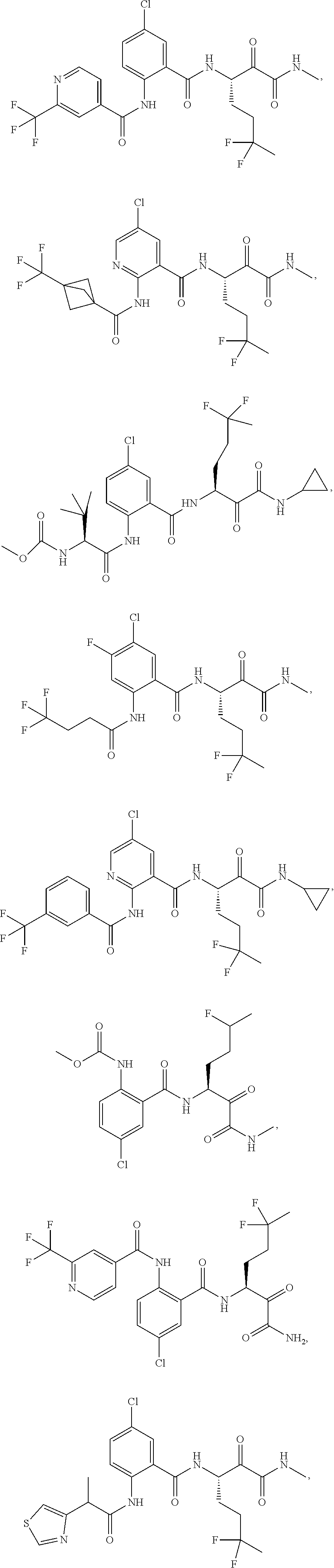

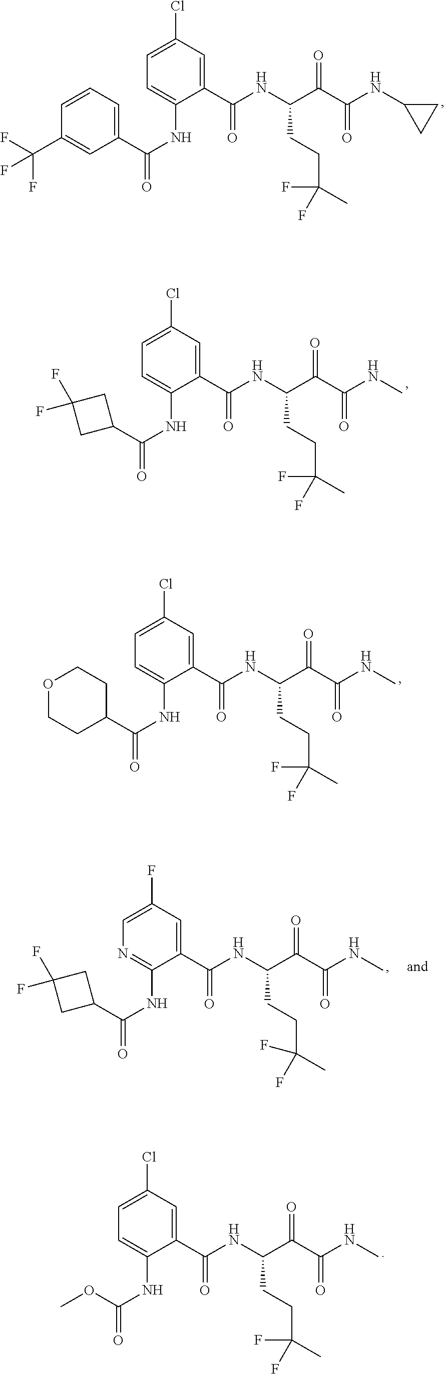

15. A pharmaceutical composition comprising the compound of claim 1 or a pharmaceutically acceptable salt thereof and a pharmaceutically acceptable carrier.

16. The pharmaceutical composition of claim 15 in the form of an orally administered tablet or capsule.

17. The pharmaceutical composition of claim 15, further comprising one or more additional therapeutic agent(s).

18. The pharmaceutical composition of claim 17, wherein the one or more additional therapeutic agent(s) are selected from molnupiravir, pomotrelvir, ensitrelvir, nirmatrelvir, and ritonavir.

19. A method for prophylaxis or treatment of a coronavirus infection, comprising administering an effective amount of the compound of claim 1 or a pharmaceutically acceptable salt thereof to a patient in need thereof.

20. The method of claim 19, wherein the coronavirus infection is a SARS-CoV, SARS-CoV-2 or MERS-CoV infection.

21. The method of claim 20, wherein the coronavirus infection is a SARS-CoV-2 infection.

22. The method of claim 19, further comprising one or more additional therapeutic agent(s) to the patient.

23. The method of claim 22, wherein the one or more additional therapeutic agent(s) are selected from molnupiravir, pomotrelvir, ensitrelvir, nirmatrelvir, and ritonavir.

24. (canceled)

25. (canceled)

26. (canceled)


SEQUENCE LISTING (1 sequence)

SEQ ID NO: 1 - protein, 13 aa, synthetic construct
  SITE 13: note = lysine-CPQ2 chromophore-amide
ESATLQSGLRKAK